(19) United States
(12) Patent Application Publication
(10) Pub. No.: US 2006/0020396 A1
(43) Pub. Date: Jan. 26, 2006

(54) Rational directed protein evolution using two-dimensional rational mutagenesis scanning

(76) Inventors: Rene Gantier, Muzy (FR); Thierry Guyon, Palaiseau (FR); Hugo Cruz Ramos, Lille (FR); Manuel Vega, Vigneux-sux-Seine (FR); Lila Drittanti, Vigneux-sur-Seine (FR)

Correspondence Address: FISH & RICHARDSON, PC, P.O. BOX 1022, MINNEAPOLIS, MN, 55440-1022, US

(21) Appl. No.: 11/196,067

(22) Filed: Aug. 2, 2005

Related U.S. Application Data
(63) Continuation of application No. 10/658,355, filed on Sep. 8, 2003.
(60) Provisional application No. 60/457,063, filed on Mar. 21, 2003.
(60) Provisional application No. 60/410,258, filed on Sep. 9, 2002.

(51) Int. Cl. G01N 33/48; G01N 33/50; C40B 30/02

(52) U.S. Cl. 702/19

(57) ABSTRACT

Rational methods for generating protein variants are provided. Processes and systems for the high throughput directed evolution of peptides and proteins employing the method are provided.

### RELATED APPLICATIONS

This application is a continuation of U.S. Ser. No. 10/658,355, filed Sep. 8, 2003, entitled “RATIONAL DIRECTED PROTEIN EVOLUTION USING TWO-DIMENSIONAL RATIONAL MUTAGENESIS SCANNING,” which claims the benefit of U.S. provisional application Ser. No. 60/457,063, filed Mar. 21, 2003, entitled “RATIONAL EVOLUTION OF CYTOKINES FOR HIGHER STABILITY, ENCODING NUCLEIC ACID MOLECULES AND RELATED APPLICATIONS;” and U.S. Provisional Application Ser. No. 60/410,258, entitled “RATIONAL EVOLUTION OF CYTOKINES FOR HIGHER STABILITY, ENCODING NUCLEIC ACID MOLECULES AND RELATED APPLICATIONS,” filed Sep. 9, 2002, each to Rene Gantier, Thierry Guyon, Hugo Cruz Ramos, Manuel Vega and Lila Drittanti.

This application is related to corresponding International PCT application No. IB03/004255, entitled RATIONAL DIRECTED PROTEIN EVOLUTION USING TWO-DIMENSIONAL RATIONAL MUTAGENESIS SCANNING. This application also is related to U.S. application Ser. No. 10/658,834, entitled “RATIONAL EVOLUTION OF CYTOKINES FOR HIGHER STABILITY, ENCODING NUCLEIC ACID MOLECULES AND RELATED APPLICATIONS,” filed Sep. 8, 2003; which claims the benefit of U.S. Provisional Application Ser. No. 60/457,135, entitled “RATIONAL EVOLUTION OF CYTOKINES FOR HIGHER STABILITY, ENCODING NUCLEIC ACID MOLECULES AND RELATED APPLICATIONS;” filed Mar. 21, 2003, and to U.S. Provisional Application Ser. No. 60/409,898, entitled “RATIONAL EVOLUTION OF CYTOKINES FOR HIGHER STABILITY, ENCODING NUCLEIC ACID MOLECULES AND RELATED APPLICATIONS,” filed Sep. 9, 2002, each to Rene Gantier, Thierry Guyon, Manuel Vega and Lila Drittanti.

The subject matter of each of the above-noted applications and provisional applications is incorporated by reference in its entirety.

### FIELD OF INVENTION

Mutant proteins having improved activities, and nucleic acid molecules encoding these proteins are provided. Uses of these proteins for treatment of diseases also are provided.

### BACKGROUND

Directed evolution refers to biotechnological processes devoted to the optimization of the protein activity by means of changes introduced into selected respective genes. Directed evolution includes the generation of a collection of mutated genes followed by the selection of mutants encoding proteins with desired features. These processes can be iterative when gene products having an improvement in a desired property are subjected to further cycles of mutation, selection and screening. The concept of mutant or mutation is used here in the wide sense of “change.” Directed evolution provides a way to adapt natural proteins to work in new chemical or biological environments, and/or to elicit new functions.

Proteins intrinsically possess an enormous potential plasticity, which allows them to face new challenges, such as a new environment and a desired new or altered activity. It is possible to take advantage of this plasticity to generate new proteins with altered activity. In a sufficiently large pool of modified mutant proteins, there is a chance of finding an appropriately modified protein having a desired activity. Problems arise, however, in generating and identifying a modified protein having a desired activity. Among the practical approaches intended to tackle these problems, two types can be distinguished. One is a purely predictive approach that is based on the assumption that the optimized proteins can be rationally designed in a predictable manner. This approach, however, requires sufficient information regarding the physiochemical properties of individual amino acids and amino acid sequences that govern protein folding, molecular interactions, intra-molecular forces and other dynamics of protein activity. The predictive approach is extremely dependent on a number of variables and parameters that are not known, even if the secondary and/or tertiary structures of a protein are available.

In contrast to the predictive approach, random or stochastic approaches have also been employed. One random approach requires synthesis of all possible protein sequences or a statistically sufficient large number of proteins followed by the screening to identify proteins having a desired activity or property. Other random approaches are based on gene shuffling methods, such as, for example, PCR-based methods that generate random rearrangements between or among two or more sequence-related genes to randomly generate variants of the original gene.

The development and scope of directed evolution, has been limited by both of the approaches described above, and its full potential remains therefore to be exploited. In order to capitalize on the full potential of directed evolution, alternative approaches for generating and identifying evolved proteins are needed. Therefore, among the objects herein, it is an object to provide methods for generating and identifying evolved proteins having desired activities.

### SUMMARY

Provided herein are methods, designated two-dimensional (2D) rational mutagenesis scanning (also referred to as 2D scanning). These methods employ an indirect search for alteration, typically improvement, of a selected activity particular activity, such as increased resistance to proteolysis or other physical or chemical property. The method uses a rational amino acid replacement and sequence change at single or a limited number of amino acid positions at a time. As a result, optimized proteins having modified amino acid sequences at some regions along the protein that perform differently from, typically better, the starting target protein. Such modified proteins are identified and isolated.

Target loci in a protein for modification are selected based on properties of the target polypeptide, including i) the particular protein properties to be evolved, ii) the protein's amino acid sequence, and iii) the known properties of the individual amino acids, a number of target amino acid positions along the protein sequence are selected in silico for replacement. The target loci (amino acid positions) along the protein sequence selected in silico for modification, typically replacement, are referred to as “is-HIT target positions.” The number of is-HIT target positions is generally selected to be as large as possible such that all reasonably possible target positions for the particular feature being evolved are identified and included. In particular embodiments less than all are identified.

The amino acids selected to replace the is-HIT target positions on the particular protein being optimized can be either all of the remaining 19 amino acids or a more restricted group of selected amino acids that are contemplated to have a desired effect on protein activity. In embodiments, where a restricted number of replacement amino acids are used, all of the amino acid positions along the protein backbone can be selected as is-HIT target positions for amino acid replacement.

To prepare the mutant proteins with replacement amino acids, mutagenesis is performed by the replacement of a single amino acid residue at one is-HIT target position on the protein backbone (e.g., “one-by-one,” such as in addressable arrays), such that each individual mutant generated is the single product of each single mutagenesis reaction. The single amino acid replacement mutagenesis reactions are repeated for each of the replacing amino acids selected at each of the is-HIT target positions. Thus, a plurality of mutant protein molecules are produced, whereby each mutant protein contains a single amino acid replacement at only one of the is-HIT target positions. Activity assessment then is individually performed on each individual protein mutant molecule, following protein expression and measurement of an activity. Preparation and identification of mutations are exemplified herein in the Examples provided herein for modification of activities of IFNα-2b. The positions in polypeptides that contain modifications that lead to a desired alteration in the targeted protein activity are referred to as LEADs.

Any protein known or otherwise available to those of skill in the art is suitable for modification of an activity or property, such as optimization of a property important for improving use as a therapeutic, using the directed evolution methods provided herein. Such proteins include, but are not limited to, including cytokines, such IFNα-2b, IFN-β and any other proteins, including those that already have been mutated or optimized by other methods.

### BRIEF DESCRIPTION OF THE FIGURES

FIG. 1(A) shows a schematic of the initial step in the methods provided herein for 2D-scanning. Once the protein feature(s) to be optimized is (are) selected (indicated as “?”), diverse sources of information or previous knowledge (i.e., protein primary, secondary or tertiary structures, literature, patents) are exploited to determine those amino acid positions that may be amenable to improved protein fitness by replacement with a different amino acid. This step utilizes protein analysis “in silico.” All possible candidate positions that might be involved in the feature being evolved are referred to herein as “in silico HITs” (“is-HITs”). The collection (or library) of all is-HITs identified during this step represents the first dimension (target residue position) of the two-dimensional scanning methods provided herein. The first dimension is restricted because only amino acids along the protein sequence that are the is-HITs.

FIG. 1(B) shows a representation of the methods provided herein to identify a collection of LEAD candidates. A series of steps is conducted, in silico as in FIG. 1A, to identify all appropriate replacing amino acids expected to improve fitness when substituted at the is-HIT positions to form candidate LEADs.

FIG. 2 shows a representation of methods provided herein for identification of LEADs. Based on the positions defined by the is-HITs and on the selected replacing amino acids (e.g., in silico candidate LEADs), a collection (library) of individual mutant molecules is produced (in vitro) such that the native amino acids at the is-HIT positions are replaced by other selected amino acids. The replacing amino acids are any of the remaining 19 amino acids so that all 20 natural amino acids are in the position, although typically they are a smaller group of selected amino acids with sets of properties appropriate to the evolving feature. Often only a subset of amino acids are used as a replacing amino acid so that the second dimension is restricted. The collection of mutant molecules, or in silico candidate LEADS, is generated, tested and phenotypically characterized one-by-one, for example, in addressable arrays. Each individual mutant in the collection is designed and produced as the single product of an independent mutagenesis reaction. Mutant molecules are such that each molecule contains one and only one mutation. Those molecules displaying improved fitness for the evolving feature are called LEADs.

FIG. 3(A) shows a further step in the methods provided herein for rational evolution of peptides and proteins. Following identification of LEADs, a new collection of mutant molecules is obtained by combination of any two or more of the mutations present in the LEAD molecules. The collection of new mutant molecules is generated, tested and phenotypically characterized such as in the one-by-one in addressable arrays exemplified in the Figure. Each individual mutant in the collection is designed and produced as the single product of an independent mutagenesis reaction. Mutant molecules are such that each molecule contains a variable number and type of LEAD mutations. Those molecules displaying further improved fitness for the evolving feature, are referred to herein as super-LEADs.

FIG. 3(B) shows an embodiment of the methods provided herein intended to redesign proteins such that they maintain levels and type of activity comparable to those of the native protein while their sequences are significantly changed by amino acid replacement. Pseudo-wild type amino acids are those amino acids that are different from the native amino acid at a given amino acid position and replace the native residue at that position without introducing any measurable change in protein activity. A population of sets of nucleic acid molecules encoding a collection of mutant molecules is generated and phenotypically characterized such that proteins with amino acid sequences different from the native ones but that still elicit the same level and type of activity as the native protein are selected.

FIG. 4 shows a schematic of the “Additive Directional Mutagenesis” (ADM) methods provided herein. ADM is a repetitive multi-step process such that at each step a new LEAD mutation is added onto the protein being evolved. The process is repeated as many times as necessary until the total number of desired mutations is introduced on the same molecule. The collection of new mutant molecules is generated, tested and phenotypically characterized one-by-one in addressable arrays. Each individual mutant in the collection is designed and produced as the single product of an independent mutagenesis reaction.

FIG. 5 depicts different levels of biological activity of a protein, designated Rep protein, super-LEADs obtained by ADM. Rep protein is is involved in replication of Adeno associated virus (see, e.g., copending U.S. application Ser. No. 10/022,390, published as US-2003-0129203-A1). It was used to exemplify the ADM method.

FIG. 6(A) displays the sequence of the mature IFNα-2b. Residues targeted by a mixture of proteases, including α-chymotrypsin (F, L, M, W, and Y), endoproteinase Arg-C(R), endoproteinase Asp-N (D), endoproteinase Glu-C (E), endoproteinase Lys-C (K), and trypsin (K, and R), are underlined and in bold lettering.

FIG. 6(B) shows the structure of IFNα-2b obtained from the NMR structure of IFNα-2a (PDB Code 1ITF) in ribbon representation. Surface residues exposed to the action of the proteases considered in FIG. 6A are in space filling representation.

FIG. 7 depicts the “Percent Accepted Mutation” (PAM250) matrix. Values given to identical residues are shown in gray squares. Highest values in the matrix are shown in black squares and correspond to the highest occurrence of substitution between two residues.

FIG. 8 presents the scores obtained from PAM250 analysis for the amino acid substitutions (replacing amino acids on the vertical axis; amino acid position on the horizontal axis) aimed at introducing resistance to proteolysis into the IFNα-2b at the protease target sequences. The two best replacing residues for each target amino acid according to the highest substitution scores are shown in black rectangles.

FIG. 9(A) depicts a zoomed portion of a tri-dimensional protein model. Both, a loop and a β-strand in the 3-dimensional (3D) structure of the protein appear to share the same neighborhood, displaying phenylalanine, cysteine and histidine residues (F, C and H in the one-letter code, respectively).

FIG. 9(B) shows the type of residue substitutions, namely F to C, H to C, and C to H, expected to allow the creation of a disulfide bond between two cysteines located in different portions of the protein. It is important to note that the sole replacement of phenylalanine by cysteine is not sufficient to form a disulfide bond due to the separating distance between replacing residues. Disulfide bonds bring rigidity to wobbling portions eventually permitting the protein to resist heating, i.e., thermostabilizing the protein.

FIG. 10(A) depicts a zoomed portion of a tri-dimensional protein model. An α-helix and a loop are linked by both a hydrogen bond and a salt bridge (dotted lines) formed between serine-histidine (S and H in the one-letter code), and arginine-glutamate residues (R and E in the one-letter code), respectively.

FIG. 10(B) shows an example of the kind of residue substitutions, namely E to A, and H to A, expected to interfere with the formation of both the hydrogen bond and the salt bridge illustrated in FIG. 10A. The lack of this linking interaction would lead to a local wobbling of protein portions, which would increase exposure of otherwise less exposed epitopes.

FIG. 11 shows a tri-dimensional model of an amphipathic polypeptide: human β-defensin (PDB code 1IJV). Its amphipathic nature is defined by the presence of two different faces in a molecule (separated by a dotted line) composed of hydrophobic and cationic (positively charged) amino acids, respectively. The positive charges of the cationic face in these amphipathic peptides are functionally important and are mainly due to arginine and/or lysine residues.

FIG. 12 illustrates the two-dimensional (2D) matrix representation of a protein sequence, wherein the vertical axis represents the amino acid present at the corresponding position indicated on the horizontal axis and the horizontal axis represents the amino acid position along the length protein sequence (such that the first cell corresponds to amino acid position No. 1, the second cell to amino acid position No. 2, etc.). The matrix always contains 20 cells in one direction (the amino acid type) and a variable number of position-cells depending on the size of the protein, the number of position-cells equaling the number of amino acids in the protein sequence. An exemplary protein sequence is shown above the matrix and within the matrix, such that those cells corresponding to the actual sequence of the protein are indicated with shaded squares.

FIG. 13(A) shows an amphipathic peptide in a 2D matrix representation, where residues in dark gray boxes and white lettering correspond to the amino acid sequence. The horizontal axis corresponds to the 37-residue sequence and the vertical axis includes the 20 amino acids in the one-letter code. A middle horizontal line separates uncharged and charged residues. The first step of one particular embodiment of the 2D-scanning methods provided herein to optimize the peptide traits also is schematized. In this particular embodiment, amino acids at all positions along the peptide sequence are sequentially replaced by either lysine or arginine residues in an attempt to further cationize and improve the amphipathic feature of the peptide. The outcome of the “Lys/Arg-scanning,” herein represented by the substitutions in the black box and white lettering, is a collection of molecules including the optimized number and positions of positive charges.

FIG. 13(B) depicts of the hypothetical combined LEADs (in light gray boxes and black lettering) resulting from the “Lys/Arg-scanning” of the peptide sequence in FIG. 13A.

FIG. 13(C) shows the next step in the 2D-scanning methods used herein to optimize the activity of the amphipathic peptide sequence in FIG. 13A. A systematic analysis corresponding to a first in silico PAM250-based analysis followed by in vitro synthesis and testing of the mutant molecules is undertaken involving each of the uncharged residues LEAD candidates (shown in black boxes and white lettering), which neighbor the previously obtained LEADs (shown in light gray boxes and black lettering).

FIG. 13(D) represents a hypothetical optimized amphipathic peptide sequence (in light gray boxes and black lettering) corresponding to a “super-LEAD” sequence, resulting from K/R scanning and mutagenesis followed by 2D-scanning (FIGS. 13B through C).

### DETAILED DESCRIPTION

A. Definitions

Unless defined otherwise, all technical and scientific terms used herein have the same meaning as is commonly understood by one of skill in the art to which the invention(s) belong. All patents, patent applications, published applications and publications, GenBank sequences, websites and other published materials referred to throughout the entire disclosure herein, unless noted otherwise, are incorporated by reference in their entirety. In the event that there is a plurality of definitions for terms herein, those in this section prevail. Where reference is made to a URL or other such identifier or address, it understood that such identifiers can change and particular information on the internet can come and go, but equivalent information can be found by searching the internet and/or is publicly available. Reference thereto evidences the availability and public dissemination of such information.

As used herein, biological activity of a protein refers to any activity manifested by the protein in vivo.

As used herein, directed evolution refers to methods that “adapt” either natural proteins, synthetic proteins or protein domains to work in new or existing natural or artificial chemical or biological environments and/or to elicit new functions and/or to increase or decrease a given activity, and/or to modulate a given feature.

As used herein, two dimensional (2D) rational mutagenesis scanning (also referred to herein as 2D-scanning) refers to the process provided herein in which two dimensions of a particular protein sequence are scanned: (1) in one dimension specific amino acid residues along the protein sequence for replacement with different amino acids are identifed; these are referred to as is-HIT target positions; and (2) in the second dimension the amino acid type for replacing a particular is-HIT target is selected, these amino acids are referred to as the replacing or replacement amino acid(s).

As used herein, in silico refers to research and experiments performed using a computer. In silico methods include, but are not limited to, molecular modeling studies, and biomolecular docking experiments.

As used herein, “is-HIT” refers to an in silico identified amino acid position along a target protein sequence that has been identified based on i) the particular protein properties to be evolved, ii) the protein's amino acid sequence, and/or iii) the known properties of the individual amino acids. These is-HIT loci on the protein sequence are identified without use of experimental biological methods. For example, once the protein feature(s) to be optimized is (are) selected, diverse sources of information or previous knowledge (i.e., protein primary, secondary or tertiary structures, literature, patents) are exploited to determine those amino acid positions that may be amenable to improved protein fitness by replacement with a different amino acid. This step utilizes protein analysis “in silico.” All possible candidate amino acid positions along a target protein's primary sequence that might be involved in the feature being evolved are referred to herein as “in silico HITs” (“is-HITs”). The collection of all is-HITs identified during this step represents the first dimension (target residue position) of the two-dimensional scanning methods provided herein.

As used herein, “amenable to providing the evolved predetermined property or activity,” in the context of identifying is-HITs, refers to an amino acid position on a target protein, based on in silico analysis, to possess properties or features that when replaced would alter the activity being evolved.

As used herein, high-throughput screening (HTS) refers to processes that test a large number of samples, such as samples of test proteins or cells containing nucleic acids encoding the proteins of interest to identify structures of interest or the identify test compounds that interact with the variant proteins or cells containing them. HTS operations are amenable to automation and are typically computerized to handle sample preparation, assay procedures and the subsequent processing of large volumes of data.

As used herein, the term “restricted,” when used in the context of the identification of is-HIT amino acid positions along the protein sequence selected for amino acid replacement and/or the identification of replacing amino acids, means that fewer than all amino acids on the protein-backbone are selected for amino acid replacement; and/or fewer than all of the remaining 19 amino acids available to replace the original amino acid present in the unmodified starting protein are selected for replacement. In particular embodiments of the methods provided herein, the is-HIT amino acid positions are restricted, such that fewer than all amino acids on the protein-backbone are selected for amino acid replacement. In other embodiments, the replacing amino acids are restricted, such that fewer than all of the remaining 19 amino acids available to replace the native amino acid present in the unmodified starting protein are selected as replacing amino acids. In a particular embodiment, both of the scans to identify is-HIT amino acid positions and the replacing amino acids are restricted, such that fewer than all amino acids on the protein-backbone are selected for amino acid replacement and fewer than all of the remaining 19 amino acids available to replace the native amino acid are selected for replacement.

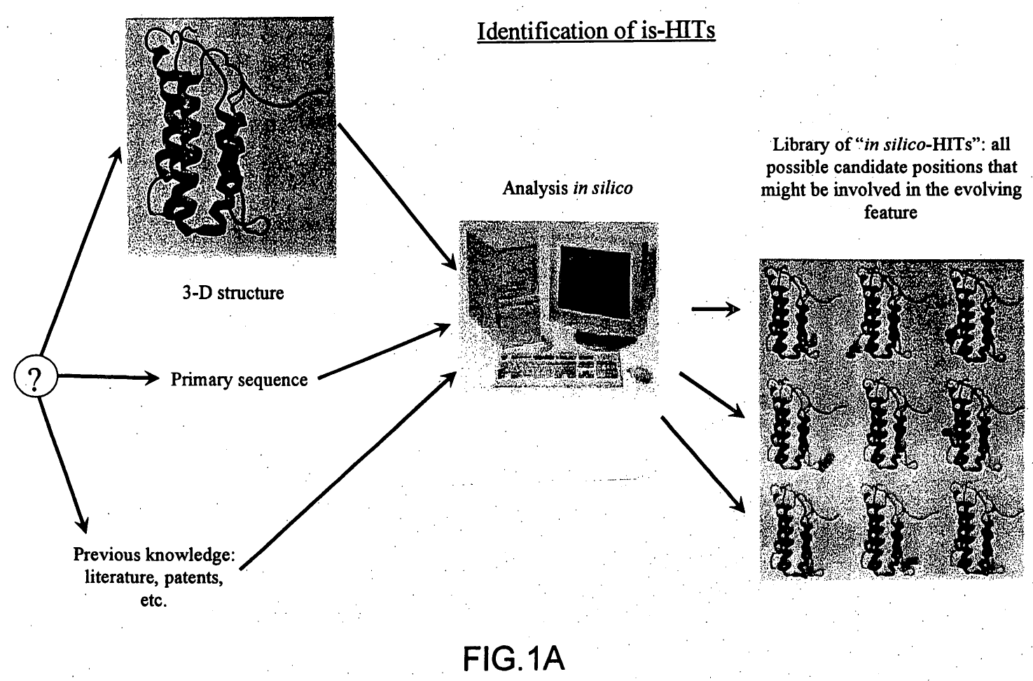

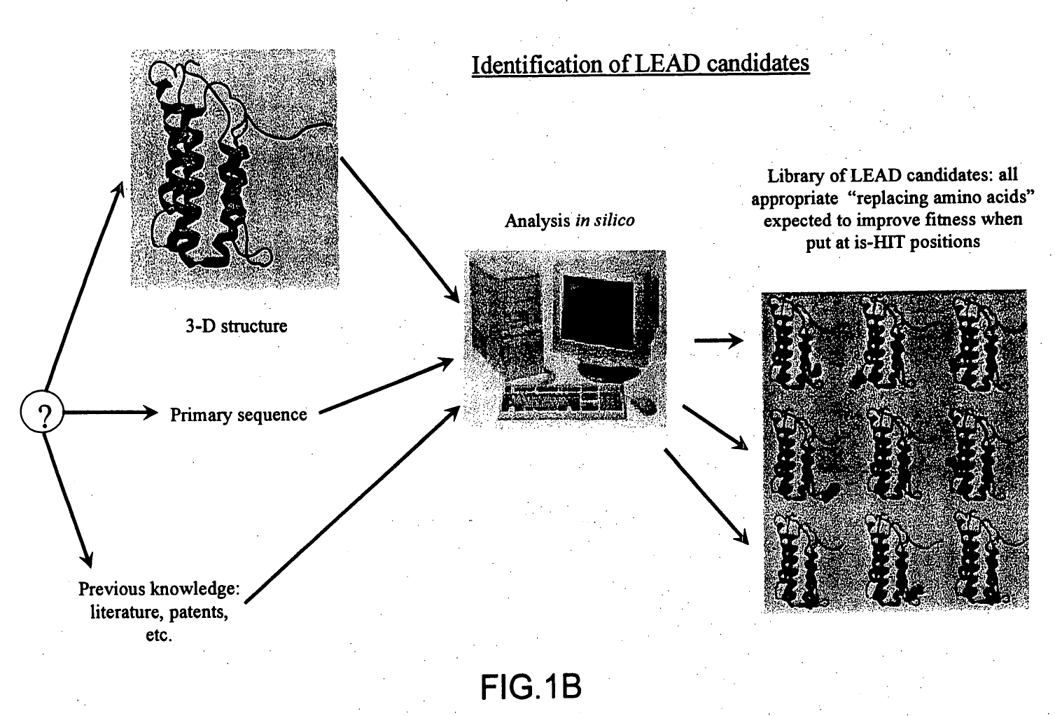

As used herein, “candidate LEADS,” are mutant proteins that are contemplated as potentially having an alteration in any attribute, chemical, physical or biological property in which such alteration is sought. In the methods herein, candidate LEADs are generally generated by systematically replacing is-HITS loci in a protein or a domain thereof with typically a restricted subset, or all, of the remaining 19 amino acids, such as obtained using PAM matrix analysis or other analysis. Candidate LEADs may be generated by other methods known to those of skill in the art tested by the high throughput methods herein (see FIG. 1B).

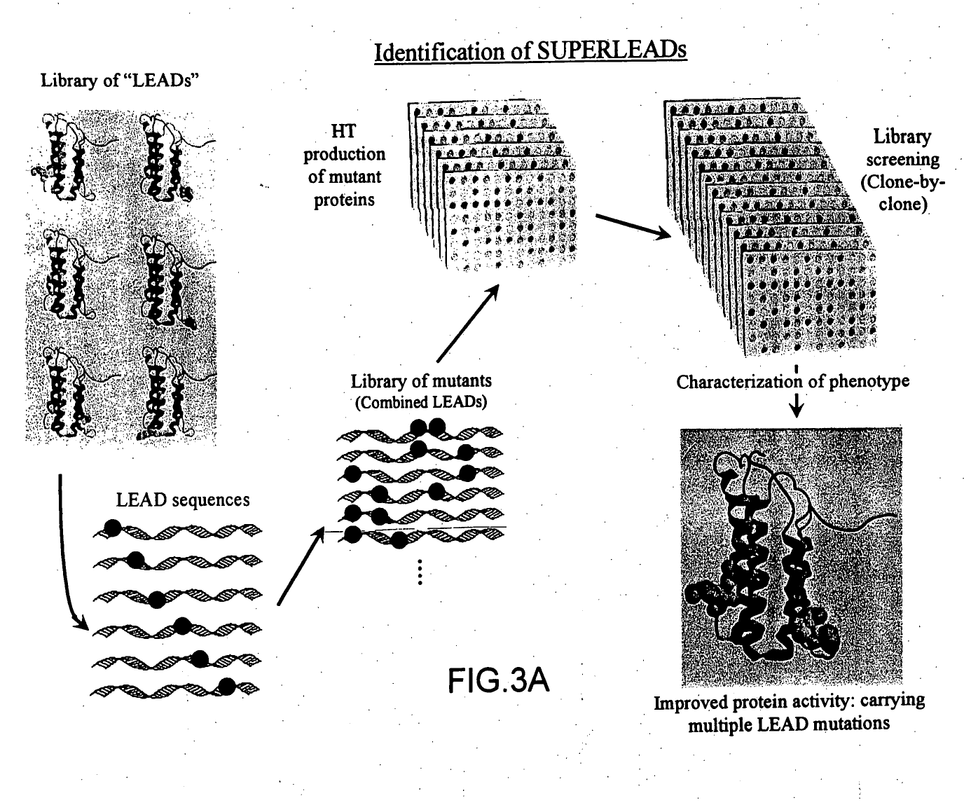

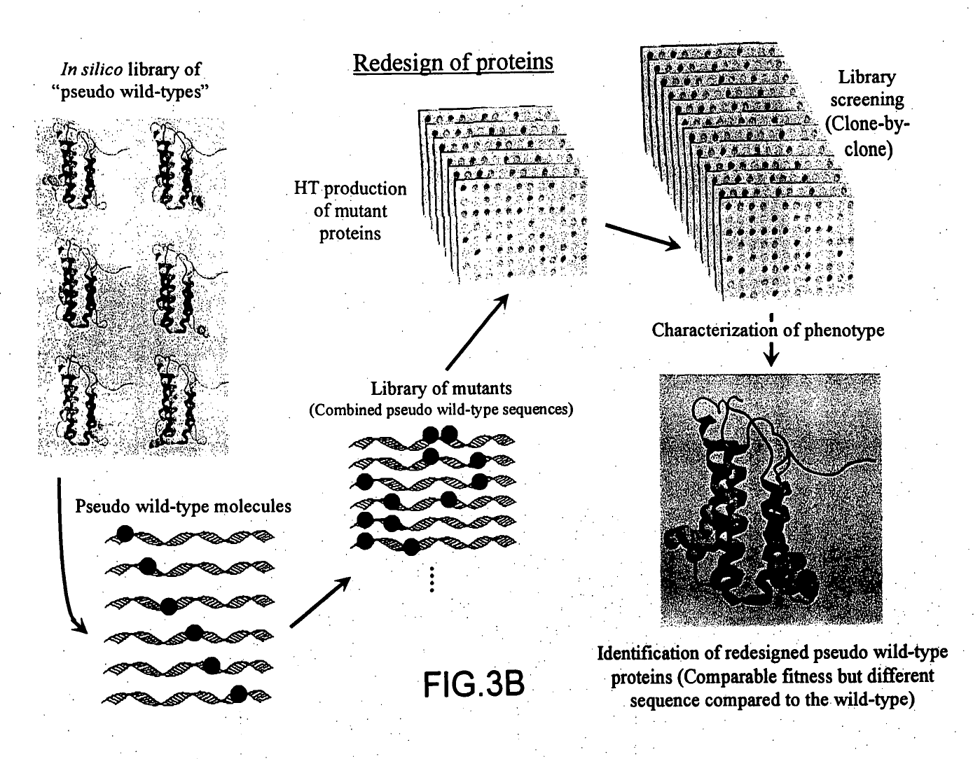

As used herein, “LEADs” are “candidate LEADs” whose activity has been demonstrated to be optimized or improved for the particular attribute, chemical, physical or biological property. For purposes herein a “LEAD” typically has activity with respect to the function of interest that differs by at least 10%, 20%, 30%, 40%, 50%, 60%, 70%, 80%, 90%, 100%, 150%, 200% or more from the unmodified and/or wild type (native) protein. In certain embodiments, the change in activity is at least about 10%, 20%, 30%, 40%, 50%, 60%, 70%, 80%, 90% or 100%, of the activity of the unmodified target protein. In other embodiments, the change in activity is not more than about 10%, 20%, 30%, 40%, 50%, 60%, 70%, 80%, 90% or 100%, of the activity of the unmodified target protein. In yet other embodiments, the change in activity is at least about 2 times, 3 times, 4 times, 5 times, 6 times, 7 times, 8 times, 9 times, 10 times, 20 times, 30 times, 40 times, 50 times, 60 times, 70 times, 80 times, 90 times, 100 times, 200 times, 300 times, 400 times, 500 times, 600 times, 700 times, 800 times, 900 times, 1000 times, or more greater than the activity of the unmodified target protein. The desired alteration, which can be either an increase or a reduction in activity, will depend upon the function or property of interest (e.g., ±10%, ±20%, etc.). The LEADs may be further optimized by replacement of a plurality (2 or more) of “is-HIT” target positions on the same protein molecule to generate “super-LEADs.” As used herein, the term “super-LEAD” refers to protein mutants (variants) obtained by combining the single mutations present in two or more of the LEAD molecules into a single protein molecule (see FIG. 3A). Accordingly, in the context of the modified proteins provided herein, the phrase “proteins comprising one or more single amino acid replacements” encompasses any combination of two or more of the mutations described herein for a respective protein. For example, the modified proteins provided herein having one or more single amino acid replacements can have can have any combination of 2, 3, 4, 5, 6, 7, 8, 9, 10, 11, 12, 13, 14, 15, 16, 17, 18, 19, 20 or more of the amino acid replacements at the disclosed replacement positions. The collection of new super-LEAD mutant molecules is generated, tested and phenotypically characterized one-by-one in addressable arrays. Super-LEAD mutant molecules are such that each molecule contains a variable number and type of LEAD mutations. Those molecules displaying further improved fitness for the particular feature being evolved, are referred to as super-LEADs. Super-LEADs may be generated by other methods known to those of skill in the art and tested by the high throughput methods herein. For purposes herein a super-LEAD typically has activity with respect to the function of interest that differs from the improved activity of a LEAD by a desired amount, such as at least 10%, 20%, 30%, 40%, 50%, 60%, 70%, 80%, 90%, 100%, 150%, 200% or more from at least one of the LEAD mutants from which it is derived. In certain embodiments, the change in activity is at least about 10%, 20%, 30%, 40%, 50%, 60%, 70%, 80%, 90% or 100%, of the activity of the unmodified target protein. In other embodiments, the change in activity is not more than about 10%, 20%, 30%, 40%, 50%, 60%, 70%, 80%, 90% or 100%, of the activity of the unmodified target protein. In yet other embodiments, the change in activity is at least about 2 times, 3 times, 4 times, 5 times, 6 times, 7 times, 8 times, 9 times, 10 times, 20 times, 30 times, 40 times, 50 times, 60 times, 70 times, 80 times, 90 times, 100 times, 200 times, 300 times, 400 times, 500 times, 600 times, 700 times, 800 times, 900 times, 1000 times, or more greater than the activity of the unmodified target protein. As with LEADs, the change in the activity for super-LEADs is dependent upon the activity that is being “evolved.” The desired alteration, which can be either an increase or a reduction in activity, will depend upon the function or property of interest.

As used herein, an exposed residue presents more than 15% of its surface exposed to the solvent.

As used herein, the phrase “unmodified target protein,” “unmodified protein” or “unmodified cytokine,” or grammatical variations thereof, refers to a starting protein that is selected for optimization using the methods provided herein. The starting unmodified target protein can be the naturally occurring, wild type form of a protein. In addition, the starting unmodified target protein may have previously been altered or mutated, such that it differs from the native wild type isoform, but is nonetheless referred to herein as an starting unmodified target protein relative to the subsequently modified proteins produced herein. Thus, existing proteins known in the art that have previously been modified to have a desired increase or decrease in a particular biological activity compared to an unmodified reference protein can be selected and used herein as the starting “unmodified target protein.” For example, a protein that has been modified from its native form by one or more single amino acid changes and possesses either an increase or decrease in a desired activity, such as resistance to proteolysis, can be utilized with the methods provided herein as the starting unmodified target protein for further optimization of either the same or a different biological activity.

As used herein, the phrase “only one amino acid replacement occurs on each target protein” refers to the modification of a target protein, such that it differs from the unmodified form of the target protein by a single amino acid change. For example, in one embodiment, mutagenesis is performed by the replacement of a single amino acid residue at only one is-HIT target position on the protein backbone (e.g., “one-by-one” in addressable arrays), such that each individual mutant generated is the single product of each single mutagenesis reaction. The single amino acid replacement mutagenesis reactions are repeated for each of the replacing amino acids selected at each of the is-HIT target positions. Thus, a plurality of mutant protein molecules are produced, whereby each mutant protein contains a single amino acid replacement at only one of the is-HIT target positions.

As used herein, the phrase “pseudo-wild type” amino acids in the context of single or multiple amino acid replacements, are those amino acids that are different from the native amino acid at a given amino acid position but can replace the native one at that position without introducing any measurable change (typically a change less than 10%, 5% or 1%, depending upon the activcity) in a particular protein activity. A population of sets of nucleic acid molecules encoding a collection of mutant molecules can be generated and phenotypically characterized such that proteins with amino acid sequences different from the native ones but that still elicit the same level and type of desired activity as the native protein can be produced.

As used herein, biological and pharmacological activity includes any activity of a biological pharmaceutical agent and includes, but is not limited to, resistance to proteolysis, biological efficiency, transduction efficiency, gene/transgene expression, differential gene expression and induction activity, titer, progeny productivity, toxicity, cytotoxicity, immunogenicity, cell proliferation and/or differentiation activity, anti-viral activity, morphogenetic activity, teratogenetic activity, pathogenetic activity, therapeutic activity, tumor suppressor activity, ontogenetic activity, oncogenetic activity, enzymatic activity, pharmacological activity, cell/tissue tropism and delivery.

As used herein, “output signal” refers to parameters that can be followed over time and, if desired, quantified. For example, when a recombinant protein is introduced into a cell, the cell containing the recombinant protein undergoes a number of changes. Any such change that can be monitored and used to assess the transformation or transfection, is an output signal, and the cell is referred to as a reporter cell; the encoding nucleic acid is referred to as a reporter gene, and the construct that includes the encoding nucleic acid is a reporter construct. Output signals include, but are not limited to, enzyme activity, fluorescence, luminescence, amount of product produced and other such signals. Output signals include expression of a gene or gene product, including heterologous genes (transgenes) inserted into the plasmid virus. Output signals are a function of time (“t”) and are related to the amount of protein used in the composition. For higher concentrations of protein, the output signal may be higher or lower. For any particular concentration, the output signal increases as a function of time until a plateau is reached. Output signals may also measure the interaction between cells, expressing heterologous genes, and biological agents.

As used herein, the activity of an IFNα-2b protein refers to any biological activity that can be assessed. In particular, herein, the activity assessed for the IFNα-2b proteins is resistance to proteolysis, antiviral activity and cell proliferation activity. As used herein, the Hill equation is a mathematical model that relates the concentration of a drug (i.e., test compound or substance) to the response measured,

$$y = \frac{{{y}_{max} [ D ]}^{x}}{{[ D ]}^{n} + {[ {D}_{50} ]}^{n}}$$

where y is the variable measured, such as a response or signal, yₘₐₓ is the maximal response achievable, [D] is the molar concentration of a drug, [D₅₀] is the concentration that produces a 50% maximal response to the drug, n is the slope parameter, which is 1 if the drug binds to a single site and with no cooperativity between or among sites. A Hill plot is log₁₀ of the ratio of ligand-occupied receptor to free receptor vs. log [D] (M). The slope is n, where a slope of greater than 1 indicates cooperativity among binding sites, and a slope of less than 1 can indicate heterogeneity of binding. This general equation has been employed for assessing interactions in complex biological systems (see, published International PCT application No. WO 01/44809 based on PCT No. PCT/FR00/03503, see, also, the EXAMPLES).

As used herein, in the Hill-based analysis (see, published International PCT application No. WO 01/44809 based on PCT No. PCT/FR00/03503), the parameters, π,κ,τ,ε,η,θ, are as follows:
π is the potency of the biological agent acting on the assay (cell-based) system;
κ is the constant of resistance of the assay system to elicit a response to a biological agent;
ε is the global efficiency of the process or reaction triggered by the biological agent on the assay system;
τ is the apparent titer of the biological agent;
θ is the absolute titer of the biological agent; and

η is the heterogeneity of the biological process or reaction.

In particular, as used herein, the parameters π (potency) or κ (constant of resistance) are used to respectively assess the potency of a test agent to produce a response in an assay system and the resistance of the assay system to respond to the agent.

As used herein, ε (efficiency), is the slope at the inflexion point of the Hill curve (or, in general, of any other sigmoidal or linear approximation), to assess the efficiency of the global reaction (the biological agent and the assay system taken together) to elicit the biological or pharmacological response.

As used herein, τ (apparent titer) is used to measure the limiting dilution or the apparent titer of the biological agent.

As used herein, θ (absolute titer), is used to measure the absolute limiting dilution or titer of the biological agent.

As used herein, η (heterogeneity) measures the existence of discontinuous phases along the global reaction, which is reflected by an abrupt change in the value of the Hill coefficient or in the constant of resistance.

As used herein, a population of sets of nucleic acid molecules encoding a collection of mutants refers to a collection of plasmids or other vehicles that carrying (encoding) the gene variants, such that individual plasmid or other vehicles carry individual gene variants. Each element of the collection (library) is physically separated from the others, individually set in an appropriate format, such asn addressable array, and is generated as a single product of an independent mutagenesis reaction. When a collection of proteins is contemplated, it will be so-stated.

As used herein, a “reporter cell” is the cell that “reports,” i.e., undergoes the change, in response to the treatment with for example a protein or a virus.

As used herein, “reporter” or “reporter moiety” refers to any moiety that allows for the detection of a molecule of interest, such as a protein expressed by a cell. Reporter moieties include, but are not limited to, for example, fluorescent proteins, such as red, blue and green fluorescent proteins; LacZ and other detectable proteins and gene products. For expression in cells, nucleic acid encoding the reporter moiety can be expressed as a fusion protein with a protein of interest or under to the control of a promoter of interest.

As used herein, phenotype refers to the physical, physiological or other manifestation of a genotype (a sequence of a gene). In methods herein, phenotypes that result from alteration of a genotype are assessed.

As used herein, “activity” means in the largest sense of the term any change in a system (either biological, chemical or physical system) of any nature (such as changes in the amount of product in an enzymatic reaction, changes in cell proliferation, in immunogenicity or in toxicity) caused by a protein or protein mutant interacting with that system. In addition, the term “activity,” “higher activity” or “lower activity” as used herein in reference to resistance to either proteases, proteolysis, incubation with serum or with blood, means the ratio or residual biological (antiviral) activity between “after” protease/blood or serum treatment and “before” protease/blood or serum treatment.

As used herein, activity refers to the function or property to be evolved. An active site refers to a site(s) responsible or that participates in conferring the activity or function. The activity or active site evolved (the function or property and the site conferring or participating in conferring the activity) may have nothing to do with natural activities of a protein. For example, it could be an ‘active site’ for conferring immunogenicity (immunogenic sites or epitopes) on a protein.

As used herein, the amino acids, which occur in the various amino acid sequences appearing herein, are identified according to their known, three-letter or one-letter abbreviations (see, Table 1). The nucleotides, which occur in the various nucleic acid fragments, are designated with the standard single-letter designations used routinely in the art.

As used herein, amino acid residue refers to an amino acid formed upon chemical digestion (hydrolysis) of a polypeptide at its peptide linkages. The amino acid residues described herein are presumed to be in the “L” isomeric form. Residues in the “D” isomeric form, which are so-designated, can be substituted for any L-amino acid residue, as long as the desired functional property is retained by the polypeptide. NH₂ refers to the free amino group present at the amino terminus of a polypeptide. COOH refers to the free carboxy group present at the carboxyl terminus of a polypeptide. In keeping with standard polypeptide nomenclature described in J. Biol. Chem., 243:3552-3559, 1969, and adopted at 37 C.F.R. §§ 1.821-1.822, abbreviations for amino acid residues are shown in Table 1:

TABLE 1
Table of Correspondence

SYMBOL

| 1-Letter | 3-Letter | AMINO ACID |
|---|---|---|
| Y | Tyr | tyrosine |
| G | Gly | glycine |
| F | Phe | phenylalanine |
| M | Met | methionine |
| A | Ala | alanine |
| S | Ser | serine |
| I | Ile | isoleucine |
| L | Leu | leucine |
| T | Thr | threonine |
| V | Val | valine |
| P | Pro | proline |
| K | Lys | lysine |
| H | His | histidine |
| Q | Gln | glutamine |
| E | Glu | glutamic acid |
| Z | Glx | Glu and/or Gln |
| W | Trp | tryptophan |
| R | Arg | arginine |
| D | Asp | aspartic acid |
| N | Asn | asparagine |
| B | Asx | Asn and/or Asp |
| C | Cys | cysteine |
| X | Xaa | Unknown or other |

It should be noted that all amino acid residue sequences represented herein by formulae have a left to right orientation in the conventional direction of amino-terminus to carboxyl-terminus. In addition, the phrase “amino acid residue” is broadly defined to include the amino acids listed in the Table of Correspondence (Table 1) and modified and unusual amino acids, such as those referred to in 37 C.F.R. §§ 1.821-1.822, and incorporated herein by reference. Furthermore, it should be noted that a dash at the beginning or end of an amino acid residue sequence indicates a peptide bond to a further sequence of one or more amino acid residues or to an amino-terminal group such as NH₂ or to a carboxyl-terminal group such as COOH.

As used herein, nucleic acids include DNA, RNA and analogs thereof, including protein nucleic acids (PNA) and mixture thereof. Nucleic acids can be single or double stranded. When referring to probes or primers, optionally labeled, with a detectable label, such as a fluorescent or radiolabel, single-stranded molecules are contemplated. Such molecules are typically of a length such that they are statistically unique of low copy number (typically less than 5, generally less than 3) for probing or priming a library. Generally a probe or primer contains at least 14, 16 or 30 contiguous of sequence complementary to or identical a gene of interest. Probes and primers can be 10, 14, 16, 20, 30, 50, 100 or more nucleic acid bases long.

Therefore, as used herein, the term “identity” represents a comparison between a test and a reference polypeptide or polynucleotide. For example, a test polypeptide may be defined as any polypeptide that is 90% or more identical to a reference polypeptide.

As used herein, the term at least “90% identical to” refers to percent identities from 90 to 100% relative to the reference polypeptides. Identity at a level of 90% or more is indicative of the fact that, assuming for exemplification purposes a test and reference polypeptide length of 100 amino acids are compared. No more than 10% (i.e., 10 out of 100) amino acids in the test polypeptide differ from that of the reference polypeptides. Similar comparisons may be made between a test and reference polynucleotides. Such differences may be represented as point mutations randomly distributed over the entire length of an amino acid sequence or they may be clustered in one or more locations of varying length up to the maximum allowable, e.g., 10/100 amino acid difference (approximately 90% identity). Differences are defined as nucleic acid or amino acid substitutions, or deletions.

As used herein, it also is understood that the terms substantially identical or similar varies with the context as understood by those skilled in the relevant art.

As used herein, a substantial change in an activity of a protein is dependent upon the activity assessed and is any that one of skill in the art would identify as not being the same activity. The same level activity is an activity that is within experimental error or expected variation for the activity.

As used herein, a therapeutically effective dose refers to that amount of the compound sufficient to result in amelioration of symptoms of disease.

A cell extract that contains the DNA or protein of interest should be understood to mean a homogenate preparation or cell-free preparation obtained from cells that express the protein or contain the DNA of interest. The term “cell extract” is intended to include culture media, especially spent culture media from which the cells have been removed.

As used herein, receptor refers to a biologically active molecule that specifically binds to (or with) other molecules. The term “receptor protein” may be used to more specifically indicate the proteinaceous nature of a specific receptor. As used herein, recombinant refers to any progeny formed as the result of genetic engineering.

As used herein, a promoter region refers to the portion of DNA of a gene that controls transcription of the DNA to which it is operatively linked. The promoter region includes specific sequences of DNA that are sufficient for RNA polymerase recognition, binding and transcription initiation. This portion of the promoter region is referred to as the promoter. In addition, the promoter region includes sequences that modulate this recognition, binding and transcription initiation activity of the RNA polymerase. These sequences may be cis acting or may be responsive to trans acting factors. Promoters, depending upon the nature of the regulation, may be constitutive or regulated.

As used herein, the phrase “operatively linked” generally means the sequences or segments have been covalently joined into one piece of DNA, whether in single or double stranded form, whereby control or regulatory sequences on one segment control or permit expression or replication or other such control of other segments. The two segments are not necessarily contiguous. For gene expression a DNA sequence and a regulatory sequence(s) are connected in such a way to control or permit gene expression when the appropriate molecular, e.g., transcriptional activator proteins, are bound to the regulatory sequence(s).

As used herein, production by recombinant means by using recombinant DNA methods means the use of the well known methods of molecular biology for expressing proteins encoded by cloned DNA, including cloning expression of genes and methods, such as gene shuffling and phage display with screening for desired specificities.

As used herein, a composition refers to any mixture of two or more products or compounds. It may be a solution, a suspension, liquid, powder, a paste, aqueous, non-aqueous or any combination thereof.

As used herein, a combination refers to any association between two or more items.

As used herein, substantially identical to a product means sufficiently similar so that the property of interest is sufficiently unchanged so that the substantially identical product can be used in place of the product.

As used herein, the term “vector” refers to a nucleic acid molecule capable of transporting another nucleic acid to which it has been linked. One type of vector is an episome, i.e., a nucleic acid capable of extra-chromosomal replication. Exemplary vectors are those capable of autonomous replication and/or expression of nucleic acids to which they are linked. Vectors capable of directing the expression of genes to which they are operatively linked are referred to herein as “expression vectors.” In general, expression vectors of utility in recombinant DNA techniques are often in the form of “plasmids” which refer generally to circular double stranded DNA loops which, in their vector form are not bound to the chromosome. “Plasmid” and “vector” are used interchangeably as the plasmid is the most commonly used form of vector. Other such other forms of expression vectors that serve equivalent functions and that become known in the art subsequently hereto.

As used herein, vector also is used interchangeable with “virus vector” or “viral vector.” In this case, which will be clear from the context, the “vector” is not self-replicating. Viral vectors are engineered viruses that are operatively linked to exogenous genes to transfer (as vehicles or shuttles) the exogenous genes into cells.

As used herein, transduction refers to the process of gene transfer and expression into mammalian and other cells mediated by viruses. Transfection refers to the process when mediated by plasmids.

As used herein, transformation refers to the process of gene transfer and expression into bacterial cells, mediated by plasmids.

As used herein, “allele,” which is used interchangeably herein with “allelic variant” refers to alternative forms of a gene or portions thereof. Alleles occupy the same locus or position on homologous chromosomes. When a subject has two identical alleles of a gene, the subject is said to be homozygous for the gene or allele. When a subject has two different alleles of a gene, the subject is said to be heterozygous for the gene. Alleles of a specific gene can differ from each other in a single nucleotide, or several nucleotides, and can include substitutions, deletions, and insertions of nucleotides. An allele of a gene also can be a form of a gene containing a mutation.

As used herein, the term “gene” or “recombinant gene” refers to a nucleic acid molecule comprising an open reading frame and including at least one exon and (optionally) an intron sequence. A gene can be either RNA or DNA. Genes may include regions preceding and following the coding region (leader and trailer). As used herein, “intron” refers to a DNA sequence present in a given gene which is spliced out during mRNA maturation.

As used herein, “nucleotide sequence complementary to the nucleotide sequence set forth in SEQ ID NO:” refers to the nucleotide sequence of the complementary strand of a nucleic acid strand having the particular SEQ ID NO:. The term “complementary strand” is used herein interchangeably with the term “complement.” The complement of a nucleic acid strand can be the complement of a coding strand or the complement of a non-coding strand. When referring to double stranded nucleic acids, the complement of a nucleic acid having a particular SEQ ID NO: refers to the complementary strand of the strand set forth in the particular SEQ ID NO: or to any nucleic acid having the nucleotide sequence of the complementary strand of the particular SEQ ID NO:. When referring to a single stranded nucleic acid having a nucleotide sequence corresponding to a particular SEQ ID NO:, the complement of this nucleic acid is a nucleic acid having a nucleotide sequence which is complementary to that of the particular SEQ ID NO:.

As used herein, the term “coding sequence” refers to that portion of a gene that encodes an amino acid sequence of a protein.

As used herein, the term “sense strand” refers to that strand of a double-stranded nucleic acid molecule that has the sequence of the mRNA that encodes the amino acid sequence encoded by the double-stranded nucleic acid molecule.

As used herein, the term “antisense strand” refers to that strand of a double-stranded nucleic acid molecule that is the complement of the sequence of the mRNA that encodes the amino acid sequence encoded by the double-stranded nucleic acid molecule.

As used herein, an array refers to a collection of elements, such as nucleic acid molecules, containing three or more members. An addressable array is one in which the members of the array are identifiable, typically by position on a solid phase support or by virtue of an identifiable or detectable label, such as by color, fluorescence, electronic signal (i.e., RF, microwave or other frequency that does not substantially alter the interaction of the molecules of interest), bar code or other symbology, chemical or other such label. In certain embodiments, the members of the array are immobilized to discrete identifiable loci on the surface of a solid phase or directly or indirectly linked to or otherwise associated with the identifiable label, such as affixed to a microsphere or other particulate support (herein referred to as beads) and suspended in solution or spread out on a surface.

As used herein, a library of molecules is a collection of molecules; the terms are used interchangeably.

As used herein, a support (also referred to as a matrix support, a matrix, an insoluble support or solid support) refers to any solid or semisolid or insoluble support to which a molecule of interest, typically a biological molecule, organic molecule or biospecific ligand is linked or contacted. Such materials include any materials that are used as affinity matrices or supports for chemical and biological molecule syntheses and analyses, such as, but are not limited to: polystyrene, polycarbonate, polypropylene, nylon, glass, dextran, chitin, sand, pumice, agarose, polysaccharides, dendrimers, buckyballs, polyacryl-amide, silicon, rubber, and other materials used as supports for solid phase syntheses, affinity separations and purifications, hybridization reactions, immunoassays and other such applications. The matrix herein can be particulate or can be in the form of a continuous surface, such as a microtiter dish or well, a glass slide, a silicon chip, a nitrocellulose sheet, nylon mesh, or other such materials. When particulate, typically the particles have at least one dimension in the 5-10 mm range or smaller. Such particles, referred collectively herein as “beads,” are often, but not necessarily, spherical. Such reference, however, does not constrain the geometry of the matrix, which may be any shape, including random shapes, needles, fibers, and elongated. Roughly spherical “beads,” particularly microspheres that can be used in the liquid phase, also are contemplated. The “beads” may include additional components, such as magnetic or paramagnetic particles (see, e.g., Dynabeads (Dynal, Oslo, Norway)) for separation using magnets, as long as the additional components do not interfere with the methods and analyses herein.

As used herein, a matrix or support particles refers to matrix materials that are in the form of discrete particles. The particles have any shape and dimensions, but typically have at least one dimension that is 100 mm or less, 50 mm or less, 10 mm or less, 1 mm or less, 100 μm or less, 50 μm or less and typically have a size that is 100 mm³ or less, 50 mm³ or less, 10 mm³ or less, and 1 mm³ or less, 100 μm³ or less and may be order of cubic microns. Such particles are collectively called “beads.”

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem., 11:942-944, 1972).

B. Directed Evolution

To date, there have been three general approaches described for protein directed evolution based on mutagenesis.

1) Pure Random Mutagenesis

Random mutagenesis methodology requires that the amino acids in the starting protein sequence are replaced by all (or a group) of the 20 amino acids. Either single or multiple replacements at different amino acid positions are generated on the same molecule, at the same time. The random mutagenesis method relies on a direct search for fitness improvement based on random amino acid replacement and sequence changes at multiple amino acid positions. In this approach neither the amino acid position (first dimension) nor the amino acid type (second dimension) are restricted; and everything possible is generated and tested. Multiple replacements can randomly happen at the same time on the same molecule. For example, random mutagenesis methods are widely used to develop antibodies with higher affinity for its ligand, by the generation of random-sequence libraries of antibody molecules, followed by expression and screening using filamentous phages.

2) Restricted Random Mutagenesis

Restricted random mutagenesis methods introduce either all of the 20 amino acids or DNA-biased residues, wherein the bias is based on the sequence of the DNA and not on that of the protein, in a stochastic or semi-stochastic manner, respectively, within restricted or predefined regions of the protein, known in advance to be involved in the biological activity being “evolved.” This method relies on a direct search for fitness improvement based on random amino acid replacement and sequence changes at either restricted or multiple amino acid positions, with the hope that a new, unpredictable amino acid sequence at specific regions would perform better than the starting sequence. In this approach the scanning can be restricted to selected amino acid positions and/or amino acid types, while material changes continue to be random in position and type. For example, the amino acid position can be restricted by prior selection of the target region to be mutated (selection of target region is based upon prior knowledge on protein structure/function); while the amino acid type is not primarily restricted as replacing amino acids are stochastically or at most “semi-stochastically” chosen. As an example, this method is used to optimize known binding sites on proteins, including hormone-receptor systems and antibody-epitope systems.

3) Non-Restricted Rational Mutagenesis

Rational mutagenesis is a two-step process and is described in co-pending U.S. application Ser. No. 10/022,249. Briefly, the first step requires amino acid scanning where all and each of the amino acids in the starting protein sequence are replaced by a third amino acid of reference (e.g., alanine). Only a single amino acid is replaced on each protein molecule at a time; while a collection of protein molecules having a single amino acid replacement is generated such that molecules are differentiated by the amino acid position at which the replacement has taken place. Mutant DNA molecules are designed, generated by mutagenesis and cloned individually, such as in addressable arrays, such that they are physically separated from each other and that each one is the single product of an independent mutagenesis reaction. Mutant protein molecules derived from the collection of mutant DNA molecules also are physically separated from each other, such as by formatting in addressable arrays.

Activity assessment on each protein molecule allows for the identification of those amino acid positions that result in a drop in activity when replaced, thus indicating the involvement of that particular amino acid position in the protein's biological activity and/or conformation that leads to fitness of the particular feature being evolved. Those amino acid positions are referred to as HITs. At the second step, a new collection of molecules is generated such that each molecule differs from each other by the amino acid present at the individual HIT positions identified in step 1. All 20 amino acids (19 amino acids and the original) are introduced at each of the HIT positions identified in step 1; while each individual molecule contains, in principle, one and only one amino acid replacement. Mutant DNA molecules are designed, generated by mutagenesis and cloned individually, such as in addressable arrays, such that they are physically separated from each other and that each one is the single product of an independent mutagenesis reaction. Mutant protein molecules derived from the collection of mutant DNA molecules also are physically separated from each other and can be formatted in addressable arrays.

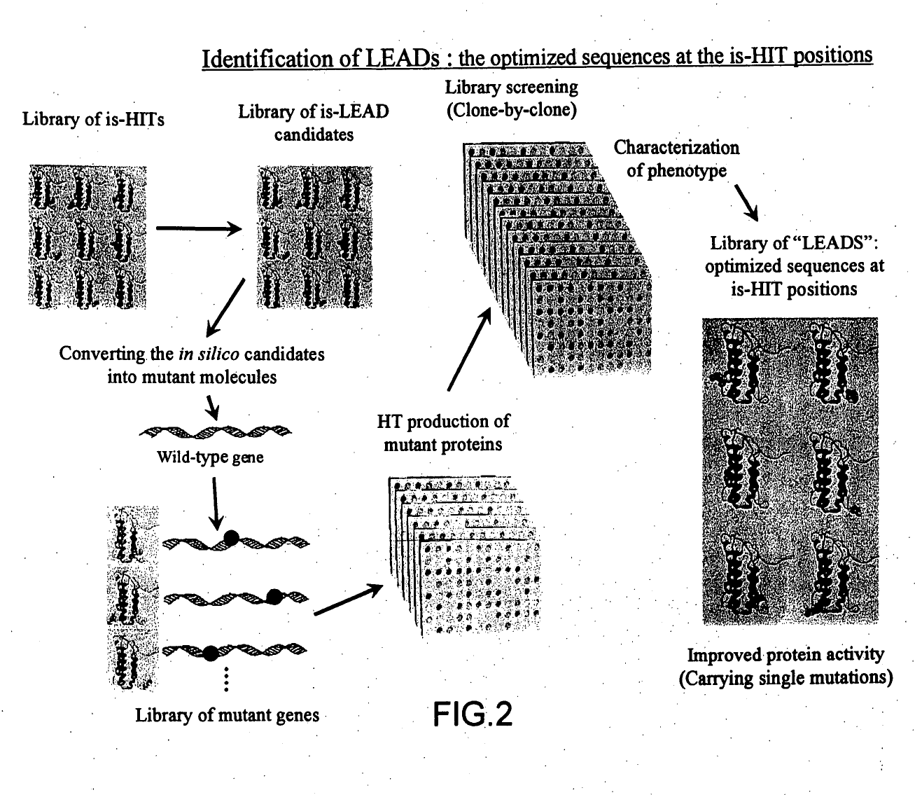

Activity assessment then is individually performed on each individual mutant molecule. The newly generated sequences that lead to an improvement in the protein activity are referred to as LEADs (FIG. 2). This method permits an indirect search for activity improvement based on one rational amino acid replacement and sequence change at single amino acid positions at a time, in search of a new, unpredictable amino acid sequence at some unpredictable regions along the protein that performs better than the starting sequence.

In this approach neither the amino acid position nor the replacing amino acid type are restricted. Full length protein scanning is performed during the first step to identify HIT positions, and then all 20 amino acids are tested at each of the HIT positions, to identify LEAD sequences; while, as a starting point, only one amino acid at a time is replaced on each molecule. The selection of the target region (HITs and surrounding amino acids) for the second step is based upon experimental data on activity obtained in the first step. Thus, no prior knowledge of protein structure and/or function is necessary. Using this approach, LEAD sequences have been found on proteins that are located at regions of the protein not previously known to be involved in the particular biological activity being optimized; thus emphasizing the power of this approach to discover unpredictable regions (HITs) as targets for fitness improvement.

C. 2-Dimensional Scanning

Provided herein are 2-Dimensional rational scanning (or “2D-scanning”) methods for protein rational evolution that are based on scanning over two dimensions: (1) one dimension is the amino acid position along the protein sequence to identify is-HIT target positions, and (2) the second dimension is the amino acid type selected for replacing the particular is-HIT amino acid position.

In particular embodiments, based on i) the particular protein properties to be evolved, ii) the protein's amino acid sequence, and iii) the known properties of the individual amino acids, a number of target positions along the protein sequence are selected, in silico, “as is-HIT target positions.” This number of is-HIT target positions is as large as possible such that all reasonably possible target positions for the particular feature being evolved are included. In particular, embodiments where a restricted number of is-HIT target positions are selected for replacement, the amino acids selected to replace the is-HIT target positions on the particular protein being optimized can be either all of the remaining 19 amino acids or, more frequently, a more restricted group comprising selected amino acids that are contemplated to have the desired effect on protein activity. In another embodiment, so long as a restricted number of replacement amino acids are used, all of the amino acid positions along the protein backbone can be selected as is-HIT target positions for amino acid replacement.

Mutagenesis then is performed by the replacement of single amino acid residues at specific is-HIT target positions on the protein backbone (e.g., “one-by-one” in addressable arrays), such that each individual mutant generated is the single product of each single mutagenesis reaction. Mutant DNA molecules are designed, generated by mutagenesis and cloned individually, in addressable arrays, such that they are physically separated from each other and that each one is the single product of an independent mutagenesis reaction. Mutant protein molecules derived from the collection of mutant DNA molecules also are physically separated from each other and can be formatted in addressable arrays. Thus, a plurality of mutant protein molecules are produced, whereby each mutant protein contains a single amino acid replacement at only one of the is-HIT target positions. Activity assessment then is individually performed on each individual protein mutant molecule, following protein expression and measurement of the appropriate activity, such as set forth in the Examples provided herein for optimization of IFNα-2b. The newly generated sequences that lead to an improvement in the protein activity are referred to as LEADs. This method relies on an indirect search for protein improvement for a particular activity, such as increased resistance to proteolysis, based on a rational amino acid replacement and sequence change at single or, in another embodiment, a limited number of amino acid positions at a time. As a result, optimized proteins having newly discovered amino acid sequences at some regions along the protein that perform better than the starting sequence are identified and isolated.

A variety of protein properties and/or biological activities can be modified using the rational mutagenesis methods provided herein, such as an increase or decrease in protein stability, the optimal pH or pH-activity of a protein, protein digestibility, protein thermostablization, protein antigenicity, the amphipathic properties of a protein, ligand-receptor interactions of a protein.

An advantage of the 2D-scanning methods provided herein is that at least one, and typically both, of the two dimensions for scanning (amino acid position and the replacing amino acid) are restricted. This means that fewer than all amino acids on the protein-backbone are selected for amino acid replacement; and/or fewer than all of the remaining 19 amino acids available to replace the original, such as native, amino acid are selected for replacement. The 2D-scanning methods provided herein are not limited to a restrictive number of selected target amino acid positions; instead the entire length of the protein is “scanned” or checked, in silico, to identify candidate amino acid positions amenable to improving the desired activity, wherein these positions are designated “in silico HITs” (“is-HITs”). Each possible amino acid and amino acid position that might be involved in the feature being evolved is identified and referred to herein as “is-HITs.” The methods provided herein are not limited to only those amino acid positions that would be the preferred candidates based on either existing algorithms, previous knowledge or intuition (this would be purely predictive). Neither do the methods provided herein replace every amino acid position along the protein (this would be purely random or stochastic). Once all the candidate amino acid positions (is-HITs) are identified, the next step involves identifying the amino acids that will be used to replace them at the respective is-HITs in the natural unmodified sequence.

Each possible amino acid that can be used as a replacing amino acid in order to evolve the selected feature while, at the same time, not having a deleterious effect on either activity or structure, is identified. The methods provided herein are not limited to a restrictive number of preferred replacing amino acids; instead all possible replacing amino acids are “tested” for each possible target position, or said the other way around, each is-HIT position is “scanned” for all possible candidate replacing amino acids. The methods are not restricted to only those amino acids that would be the preferred candidates based on existing algorithms, knowledge or intuition (this would be purely predictive). Neither do the methods provided herein replace every one of the remaining 19 amino acids as replacing amino acids (this would be purely random or stochastic).

To compare the 2D-scanning methods provided herein to the “Pure Random Mutagenesis,” “Restricted Random Mutagenesis” and “Rational Mutagenesis” methods described above, the following example in which enzyme activity at a pH different from the optimal pH for the native protein is improved is considered. The object is to identify mutants in which specific amino acid replacement(s) lead to a shift in the pH profile of the enzyme.

The “pure random mutagenesis” approach would proceed by blinded random (stochastic) amino acid replacement at any place on the protein sequence, whether the protein 3-dimensional structure is known or not. The “restricted random mutagenesis” approach, however, in the absence of knowledge about the 3-dimensional structure. Where where the 3-dimensional structure of the protein is known, this method joins and becomes a sort of “pure random mutagenesis” approach.

In a rational mutagenesis” approach, an amino acid-scanning step would be performed, in order to identify those amino acid positions (HITs) that would be involved in the determination of the optimal pH. As the outcome of the second step, suitable amino acids would have been identified such that when put at the HIT positions lead to a change in optimal pH.

In the example of the enzyme pH activity profile, in practicing the “2D-scanning” methods provided those amino acid positions (the “is-HITs”) that may either affect optimal pH or are otherwise related to pH-activity are identified. This is done solely based on the primary amino acid sequence. In the example, the is-HITs will, in principle, be located at every position along the protein sequence where there is an amino acid susceptible to be either proton donor or proton acceptor. Each and every one of those amino acids is considered potentially involved in the determination of the optimal pH. No other assumptions are made. These is-HITs are chosen independently from any assumptions based on protein structure; the choice, in the example, is based only on intrinsic properties of the individual amino acids. These amino acids positions (target positions) are taken to the next step in the process as is-HITs.

At the second step, a collection of physical (i.e., this step is not “in silico”) “candidate LEAD” mutant molecules is generated such that each candidate LEAD molecule differs from each other by the amino acid present at one or more is-HIT positions. In certain embodiments, all 20 amino acids may be introduced at each of the is-HIT positions; while each individual molecule contains, in principle, either only one or a few amino acid replacements at different is-HIT positions. In another embodiment, only a restricted group of amino acids could be used to replace the original amino acids at the is-HIT positions. These replacing amino acids are chosen based on their intrinsic properties: i.e., in our example of the optimal pH, the subset of replacing amino acids would be restricted to only those amino acids able to function as either a proton donor or a proton receptor.

The 2D rational scanning methods provided herein still maintain the value of performing a “blinded” screening, that is observed in the other three approaches; although it is more conditioned by previous knowledge of amino acid properties, in the sense that it relies on a higher number of assumptions and hypotheses. This effect is partially countered by the fact that as many alternative is-HIT positions as possible, identified based on different criteria (helix-turn disruption, hydrophobicity, and other parameters), are covered. On the other hand, the number of different replacing amino acids is kept as large as reasonably possible, up to all the 20 amino acids (at each position), whenever appropriate. Despite of the restrictions introduced by the rational assumptions made in the choice of is-HIT target positions and of the replacing amino acids, because the selection of both is-HIT target positions and replacing amino acids is limited to a minimum (keeping the number of is-HIT as large as possible) and the replacing amino acid type as broad as possible, the 2D-scanning method provided herein is extremely rich in its potential for exploring unexpected and innovative amino acid sequences, while at the same time, being highly efficient in terms of attrition rate between mutants generated and LEAD molecules obtained. Given the number of different candidate LEAD protein molecules that are generated (e.g., a few thousands per collection), a high-throughput screening is typically necessary.

1) Identifying In-Silico HITs

Provided herein is a method for directed evolution that includes identifying and selecting (using in silico analysis) specific amino acids and amino acid positions (referred to herein as is-HITs; see, e.g., FIG. 1A) along the residues in a protein that are contemplated to be directly or indirectly involved in a feature being evolved. The 2D-scanning methods provided herein use the following two-steps. The first step is an in silico search on the particular protein's amino acid sequence to identify all possible amino acid positions that can potentially be targets for the activity being evolved. This is effected, for example, by assessing the effect of amino acid residues on the property or properties to be altered on the protein, using standard software. The particulars of the in silico analysis is a function of the property to be modified. For example, as provided herein, the property improved is the resistance of a protein to proteolysis. To determine amino acid residues that are potential targets as is-HITs, in this example, all possible target residues for proteases are first identified. The 3-dimensional structure of the protein is the considered in order to identify surface residues. Comparison of exposed residues with proteolytically cleavable residues yields residues that are targets for change.

Once identified, these amino acid positions or target sequences are referred to as “is-HITs” (in silico HITs; FIG. 1A). In silico HITs are defined as those amino acid positions (or target positions) that potentially are involved in the “evolving” feature, such as increased resistance to proteolysis. In one embodiment, the discrimination of the is-HITs among all the amino acid positions in a protein sequence is made based on i) the amino acid type at each position in addition to, whenever available but not necessarily, ii) the information on the protein secondary or tertiary structure. In silico HITs constitute a collection of mutant molecules such that all possible amino acids, amino acid positions or target sequences potentially involved in the evolving feature are represented. No strong theoretical discrimination among amino acids or amino acid positions is made at this stage.

In silico HIT positions are spread over the full length of a protein sequence. In one embodiment, only one single is-HIT amino acid at a time is replaced on the target protein. In another embodiment, a limited number of is-HIT amino acids are replaced at the same time on the same target protein molecule. The selection of target regions (is-HITs and surrounding amino acids) for the second step is based upon rational assumptions and predictions. No prior knowledge of protein structure/function is necessary. In some embodiments, the use of the 2D-scanning methodology provided herein does not necessarily require any previous knowledge of the 3-dimensional conformational structure of the protein.

Any protein known or otherwise available to those of skill in the art is suitable for optimization using the directed evolution methods provided herein, including cytokines (e.g., IFNα-2b) or any other proteins that have already been mutated or optimized.

A variety of parameters can be analyzed to determine whether or not a particular amino acid on a protein might be involved in the evolving feature. For example, the information provided by crystal structures of proteins can be rationally exploited in order to perform a computer-assisted (in silico) analysis towards the prediction of variants with desired features. In a particular embodiment, a limited number of initial premises (typically no more than 2) are used to determine the in silico HITS. In other embodiments, the number of premises used to determine the in silico HITs can range from 1 to 10 premises, including no more than 9, no more than 8, no more than 7, no more than 6, no more than 5, no more than 4, no more than 3, but are typically no more than 2 premises. It is important to the methods provided herein that the number of initial premises be kept to a minimum, so as to maintain the number of potential is-HITs at a maximum (here is where the methods provided are not limited by too much prediction based on theoretical assumptions). When two premises are employed, the first condition is typically the amino acid type itself, which is directly linked to the nature of the evolving feature. For example, if the goal were to change the optimum pH for an enzyme, then the replacing-amino acids selected at this step for the replacement of original sequence would be only those with a certain pKa value. The second premise is typically related to the specific position of those amino acids along the protein structure. For example, some amino acids might be discarded if they are not expected to be exposed enough to the solvent, even when they might have appropriate pKa values.

During the first step of identification of is-HITs according to the methods provided herein, each individual amino acid along the protein sequence is considered individually to assess whether it is a candidate for is-HIT. This search is done one-by-one and the decision on whether the amino acid is considered to be a candidate for a is-HIT is based on (1) the amino acid type itself; (2) the position on the amino acid sequence and protein structure if known; and (3) the predicted interaction between that amino acid and its neighbors in sequence and space.

In an additional embodiment, once one protein within a family of proteins (e.g., IFNα-2b within the cytokine family) is optimized using the methods provided herein for generating LEAD mutants, is-HITs can be readily identified on the remaining proteins within the particular family by identifying the corresponding amino acid positions therein using a structural homology analysis (see, co-pending U.S. application Ser. No. 922, filed the same day herewith, based on U.S. Provisional Application Ser. No. 60/457,135 and to U.S. Provisional Application Ser. No. 60/409,898). The is-HITs identified in this manner then can be subjected to the next step of identifying replacing amino acids and further assayed to obtain LEADs or super-LEADs as described herein.

2) Identifying Replacing Amino Acids

Once the is-HITs target positions (target loci) have been selected, the next step is identifying those amino acids that will replace the original, such as native, amino acid at each is-HIT position to alter the activity level for the particular feature being evolved. The set of replacing amino acids to be used to replace the original, such as native, amino acid at each is-HIT position can be different and specific for the particular is-HIT position. The choice of the replacing amino acids takes into account the need to preserve the physicochemical properties such as hydrophobicity, charge and polarity, of essential (e.g., catalytic, binding, etc.) residues. The number of replacing amino acids, of the remaining 19 non-native (or non-original) amino acids, that can be used to replace a particular is-HIT target position ranges from 1 up to about 19, from 1 up to about 15, from 1 up to about 10, from 1 up to about 9, from 1 up to about 8, from 1 up to about 7, from 1 up to about 6, from 1 up to about 5, from 1 up to about 4, from 1 up to about 3, or from 1 to 2 amino acid replacements.

Numerous methods of selecting replacing amino acids are well known in the art. Protein chemists determined that certain amino acid substitutions commonly occur in related proteins from different species. As the protein still functions with these substitutions, the substituted amino acids are compatible with protein structure and function. Often, these substitutions are to a chemically similar amino acid, but other types of changes, although relatively rare, also can occur.

Knowing the types of changes that are most and least common in a large number of proteins can assist with predicting alignments and amino acid substitutions for any set of protein sequences. Amino acid substitution matrices are used for this purpose.

In amino acid substitution matrices, amino acids are listed across the top of a matrix and down the side, and each matrix position is filled with a score that reflects how often one amino acid would have been paired with the other in an alignment of related protein sequences. The probability of changing amino acid A into amino acid B is assumed to be identical to the reverse probability of changing B into A. This assumption is made because, for any two sequences, the ancestor amino acid in the phylogenetic tree is usually not known. Additionally, the likelihood of replacement should depend on the product of the frequency of occurrence of the two amino acids and on their chemical and physical similarities. A prediction of this model is that amino acid frequencies will not change over evolutionary time (Dayhoff et al., Atlas of Protein Sequence and Structure, 5 (3):345-352, 1978). Below are several exemplary amino acid substitution matrices, including, but not limited to block substitution matrix (BLOSUM), Jones, Gonnet, Fitch, Feng, McLachlan, Grantham, Miyata, Rao, Risler, Johnson and percent accepted mutation (PAM). Any such method known to those of skill in the art can be employed.

(a) Percent Accepted Mutation (PAM)

Dayhoff and coworkers developed a model of protein evolution that resulted in the development of a set of widely used replacement matrices (Dayhoff et al., Atlas of Protein Sequence and Structure, 5 (3):345-352, 1978) termed percent accepted mutation matrices (PAM). In deriving these matrices, each change in the current amino acid at a particular site is assumed to be independent of previous mutational events at that site. Thus, the probability of change of any amino acid A to amino acid B is the same, regardless of the previous changes at that site and also regardless of the position of amino acid A in a protein sequence.

In the Dayhoff approach, replacement rates are derived from alignments of protein sequences that are at least 85% identical; this constraint ensures that the likelihood of a particular mutation being the result of a set of successive mutations is low. Because these changes are observed in closely related proteins, they represent amino acid substitutions that do not significantly change the function of the protein. Hence, they are called “accepted mutations,” as defined as amino acid changes that are accepted by natural selection.

(i) PAM Analysis

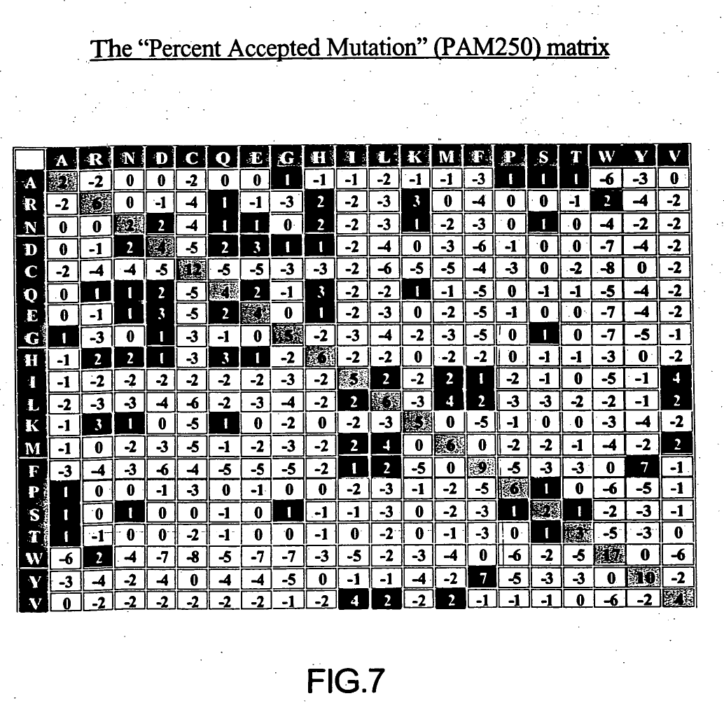

In particular embodiments of the methods provided herein, “Percent Accepted Mutation” (PAM; Dayhoff et al., Atlas of Protein Sequence and Structure, 5 (3):345-352, 1978, FIG. 7) PAM values are used to select an appropriate group of replacement amino acids. PAM matrices were originally developed to produce alignments between protein sequences based evolutionary distances (see FIG. 7). Because, in a family of proteins or homologous (related) sequences, identical or similar amino acids (85% similarity) are shared, conservative substitutions for, or “allowed point mutations” of the corresponding amino acid residues can be determined throughout an aligned reference sequence. In this regard, “conservative substitutions” of a residue in a reference sequence are those substitutions that are physically and functionally similar to the corresponding reference residues, e.g., that have a similar size, shape, electric charge, chemical properties, including the ability to form covalent or hydrogen bonds, or the like. Particularly suitable conservative amino acid substitutions are those that show the highest scores and fulfill the PAM matrix criteria in the form of “accepted point mutations.” For example, by comparing a family of scoring matrices, Dayhoff et al., Atlas of Protein Sequence and Structure, 5 (3):345-352, 1978, found a consistently higher score significance when using PAM250 matrix to analyze a variety of proteins, known to be distantly related.

(ii) PAM 250

In a particular embodiment, the PAM250 matrix set forth in FIG. 7 is used for determining the replacing amino acids based on “similarity” criteria. The PAM250 matrix uses data obtained directly from natural evolution to facilitate the selection of replacing amino acids for the is-HITs to generate conservative mutations without much affecting the overall protein function. By using the PAM250 matrix, candidate replacing amino acids are identified from related proteins from different organisms.

(b) Jones and Gonnet

This method (see, e.g., Jones et al., Comput. Appl. Biosci., 8:275-282, 1992 and Gonnet et al., Science, 256:1433-1445, 1992) uses much of the same methodology as Dayhoff (see below), but with modern databases. The matrix of Jones et al., is extracted from Release 15.0 of the SWISS-PROT protein sequence database. Point mutations totaling 59,160 from 16,130 protein sequences were used to calculate a PAM250 (see below) matrix.

The matrix published by Gonnet et al., Science, 256:1433-1445, 1992, was built from a sequence database of 8,344,353 amino acid residues. Each sequence was compared against the entire database, such that 1.7×10⁶ subsequent matches resulted for the significant alignments. These matches were then used to generate a matrix with a PAM distance of 250.

(c) Fitch and Feng

Fitch, J. Mol. Evol., 16 (1):9-16, 1966 used an exchange matrix that contained for each pair (A, B) of amino acid types the minimum number of nucleotides that must be changed to encode amino acid A instead of amino acid B. Feng et al., J. Mol. Evol., 21:112-125, 1985, used an enhanced version of Fitch, J. Mol. Evol., 16 (1):9-16, 1966, to build a Structure-Genetic matrix. In addition to considering the minimum number of base changes required to encode amino acid B instead of A, this method also considers the structural similarity of the amino acids.

(d) McLachlan, Grantham and Miyata

McLachlan, J. Mol. Biol, 61:409-424 1971, used 16 protein families, each with 2 to 14 members. The 89 sequences were aligned and the pairwise exchange frequency, observed in 9280 substitutions, was used to generate an exchange matrix with values varying from 0 to 9.

Grantham, Science, 185:862-864, 1974, considers composition, polarity and molecular volume of amino acid side-chains, properties that were highly correlated to the relative substitution frequencies tabulated by McLachlan, J. Mol. Biol., 61:409-424, 1971, to build the matrix.

Miyata, J. Mol. Evol, 12:219-236, 1979, uses the volume and polarity values of amino acids published by Grantham, Science, 185:862-864, 1974. For every amino acid type pair, the difference for both properties was calculated and divided by the standard deviation of all the differences. The square root of the sum of both values then is used in the matrix.

(e) Rao

Rao, J. Pept. Protein Res., 29:276-281, 1987, employs five amino acid properties to create a matrix; namely, alpha-helical, beta-strand and reverse-turn propensities as well as polarity and hydrophobicity. The standardized properties were summed and the matrix rescaled to the same average as that for PAM (Dayhoff et al., Atlas of Protein Sequence and Structure, 5 (3):345-352, 1978).

(f) Risler

Risler et al., J. Mol. Biol, 204:1019-1029, 1988, aligned 32 three-dimensional structures from 11 protein families by rigid-body superposition of the backbone topology. Only substitutions were considered where at least three adjacent and equivalent main-chain C alpha atom pairs in the compared structures were each not more than 1.2 Å apart. A total of 2860 substitutions were considered and used to build a matrix based on χ² distance calculations.

(g) Johnson

Johnson et al., J. Mol. Biol., 233:716-738, 1993, derived their matrix from the tertiary structural alignment of 65 families in a database of 235 structures created with the method of Sali et al., J. Mol. Biol., 212:403-428, 1990. Their examination of the substitutions was based on the expected and observed ratios of occurrences and the final matrix values were taken as log₁₀ of the ratios.

(h) Block Substitution Matrix (BLOSUM)

One empirical approach (Henikoff et al., Proc. Natl. Acad. Sci. USA, 89:10915-10919, 1992) uses local, ungapped alignments of distantly related sequences to derive the blocks amino acid substitution matrix (BLOSUM) series of matrices. The matrix values are based on the observed amino acid substitutions in a larger set of about 2000 conserved amino acid patterns, termed blocks. These blocks act as signatures of families of related proteins. Matrices of this series are identified by a number after the matrix (e.g., BLOSUM50), which refers to the minimum percentage identity of the blocks of multiple aligned amino acids used to construct the matrix. It is noteworthy that these matrices are directly calculated without extrapolations, and are analogous to transition probability matrices P(T) for different values of T, estimated without reference to any rate matrix Q.

The outcome of these two steps set forth above, which is performed in silico is that: (1) the amino acid positions that will be the target for mutagenesis are identified; these positions are referred to as is-HITs; (2) the replacing amino acids for the original, such as native, amino acids at the is-HITs are identified, thus providing a collection (library) of candidate LEAD mutant molecules that are expected to perform better than the native one and that are assayed for the desired optimized biological activity.

3) Physical Construction of Mutant Proteins and Biological Assays

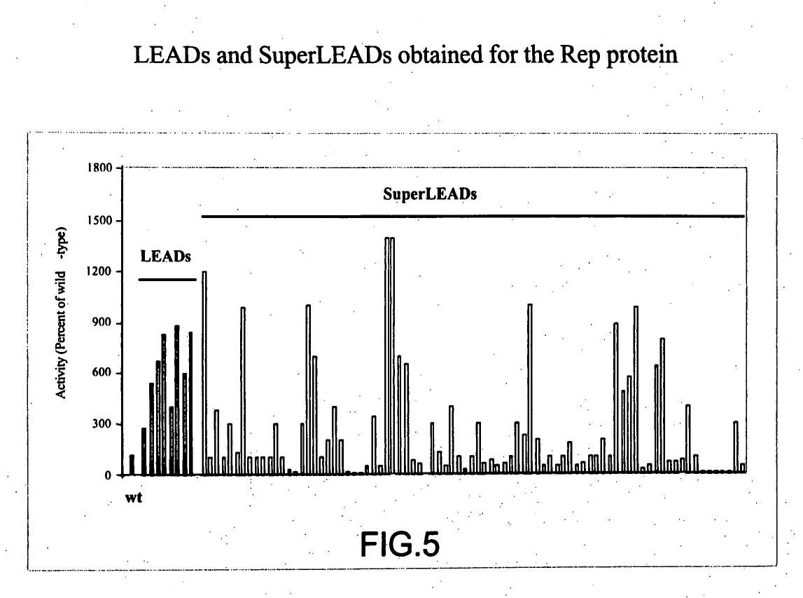

Once is-HITs are selected as set forth above, replacing amino acids are introduced. Mutant proteins typically are prepared using recombinant DNA methods and assessed in appropriate biological assays for the particular biological activity (feature) optimized (see, e.g., Example 1 and FIG. 5). An exemplary method of preparing the mutant proteins is by mutagenesis of the original, such as native, gene using methods well known in the art. Mutant molecules are generated one-by-one, such as in addressable arrays, such that each individual mutant generated is the single product of each single and independent mutagenesis reaction. Individual mutagenesis reactions are conducted such that they are physically separated from each other, for example, in addressable arrays. Once a population of sets of nucleic acid molecules encoding the respective mutant proteins is prepared, they are transfected one-by-one into appropriate cells for the production of the corresponding mutant proteins. This also can be performed in addressable arrays where each set of nucleic acid molecules encoding a respective mutant protein is introduced into cells confined to a discrete location, such as in a well of a multi-well microtiter plate. Each individual mutant protein is individually phenotypically characterized and performance is quantitatively assessed using assays appropriate for the feature being optimized (i.e., feature being evolved). Again, this step can be performed in addressable arrays. Those mutants displaying a desired increased or decreased performance compared to the original, such as native molecules are identified and designated LEADs.

From the beginning of the process of generating the mutant DNA molecules up through the readout and analysis of the performance results, each candidate LEAD mutant can be generated, produced and analyzed individually from its own address in an addressable array.

D. Super-LEADs and Additive Directed Mutagenesis (ADM).

Also provided herein are methods for generating super-LEAD mutant proteins and exemplary resulting super-LEAD mutant products. Super-LEAD mutant proteins contain a combination of single amino acid mutations present in two or more of the respective LEAD mutant proteins. The LEAD mutant proteins can be generated by the 2D scanning methods provided herein or by other methods known to those of skill in the art.

Super-LEAD mutant proteins have two of more of the single amino acid mutations derived from two or more of the respective LEAD mutant proteins. As described herein, LEAD mutant proteins provided are defined as mutants whose performance or fitness has been optimized with respect to the native protein. LEADs typically contain one single mutation relative to its respective native protein. This mutation represents an appropriate amino acid replacement that takes place at one is-HIT position. Super-LEAD mutant proteins are created such that they carry on the same protein molecule, more than one LEAD mutation, each at a different is-HIT position (see FIG. 3A). In one embodiment, once the LEAD mutant proteins have been identified using the 2D-scanning methods provided herein, super-LEADs can be generated by combining two or more individual LEAD mutant mutations using any method known in the art. These methods, include recombination, mutagenesis and DNA shuffling and any others known to those of skill in the art and/or provided herein, such as additive directional mutagenesis and multi-overlapped primer extensions.

1) Additive Directional Mutagenesis.

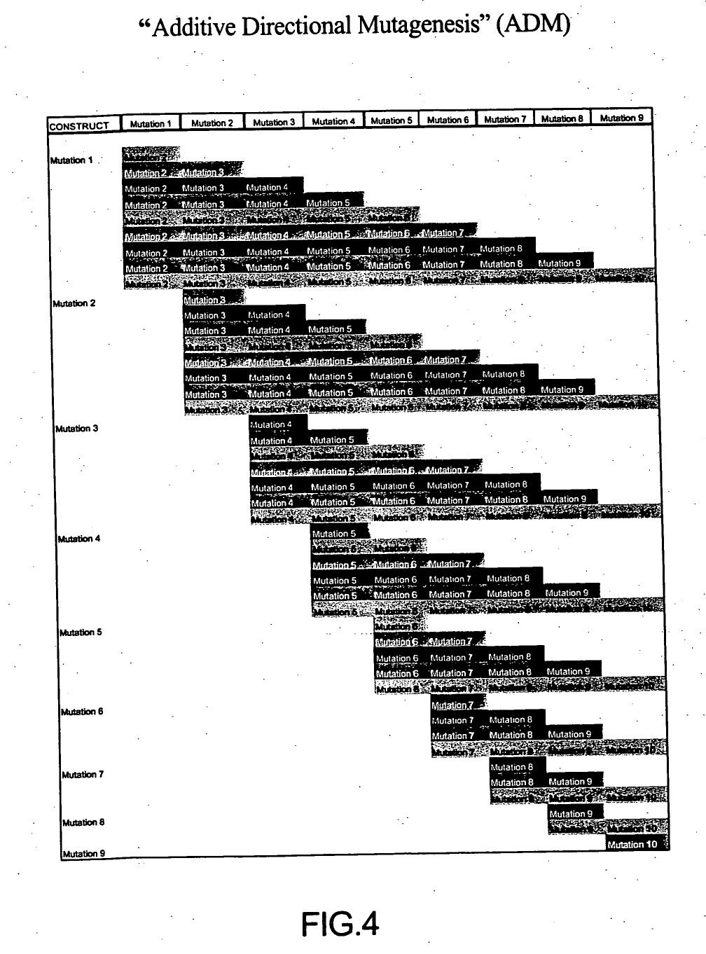

Also provided herein are methods for assembling on a single mutant protein multiple mutations present on the individual LEAD molecules, so as to generate super-LEAD mutant proteins. This method is referred to herein as “Additive Directional Mutagenesis” (ADM; see FIG. 4). ADM comprises a repetitive multi-step process where at each step after the creation of the first LEAD mutant protein a new LEAD mutation is added onto the previous LEAD mutant protein to create successive super-LEAD mutant proteins. ADM is not based on genetic recombination mechanisms, nor on shuffling methodologies; instead it is a simple one-mutation-at-a-time process, repeated as many times as necessary until the total number of desired mutations is introduced on the same molecule. To avoid the exponentially increasing number of all possible combinations that can be generated by putting together on the same molecule a given number of single mutations, a method is provided herein that, although it does not cover all the combinatorial possible space, still captures a big part of the combinatorial potential. The word “combinatorial” is used here in its mathematical meaning (i.e., subsets of a group of elements, containing some of the elements in any possible order) and not in the molecular biological or directed evolution meaning (i.e., generating pools, or mixtures, or collections of molecules by randomly mixing their constitutive elements).

A population of sets of nucleic acid molecules encoding a collection of new super-LEAD mutant molecules is generated, tested and phenotypically characterized one-by-one in addressable arrays. super-LEAD mutant molecules are such that each molecule contains a variable number and type of LEAD mutations. Those molecules displaying further improved fitness for the particular feature being evolved, are referred to as super-LEADs. Super-LEADs may be generated by other methods known to those of skill in the art and tested by the high throughput methods herein. For purposes herein a super-LEAD typically has activity with respect to the function or biological activity of interest that differs from the improved activity of a LEAD by a desired amount, such as at least 10%, 20%, 30%, 40%, 50%, 60%, 70%, 80%, 90%, 100%, 150%, 200% or more from at least one of the LEAD mutants from which it is derived. In yet other embodiments, the change in activity is at least about 2 times, 3 times, 4 times, 5 times, 6 times, 7 times, 8 times, 9 times, 10 times, 20 times, 30 times, 40 times, 50 times, 60 times, 70 times, 80 times, 90 times, 100 times, 200 times, 300 times, 400 times, 500 times, 600 times, 700 times, 800 times, 900 times, 1000 times, or more greater than at least one of the LEAD molecules from which it is derived. As with LEADs, the change in the activity for super-LEADs is dependent upon the activity that is being “evolved.” The desired alteration, which can be either an increase or a reduction in activity, will depend upon the function or property of interest.

In one embodiment provided herein, the ADM method employs a number of repetitive steps, such that at each step a new mutation is added on a given molecule. Although numerous different ways are possible for combining each LEAD mutation onto a super-LEAD protein, an exemplary way the new mutations (e.g., mutation 1 (m1), mutation 2 (m2), mutation 3 (m3), mutation 4 (m4), mutation 5 (m5), mutation n (mn)) can be added corresponds to the following diagram:
m1
m1+m2
m1+m2+m3
m1+m2+m3+m4
m1+m2+m3+m4+m5
m1+m2+m3+m4+m5+ . . . +mn
m1+m2+m4
m1+m2+m4+m5
m1+m2+m4+m5+ . . . +mn
m1+m2+m5
m1+m2+m5+ . . . +mn
m2
m2+m3
m2+m3+m4
m2+m3+m4+m5
m2+m3+m4+m5+ . . . +mn
m2+m4
m2+m4+m5
m2+m4+m5+ . . . +mn
m2+m5
m2+m5+ . . . +mn
. . . , etc. . . .

2) Multi-Overlapped Primer Extensions.

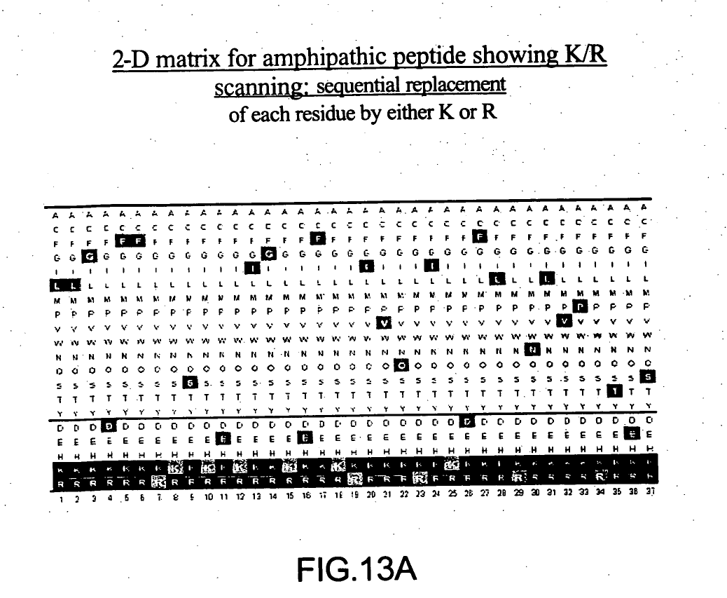

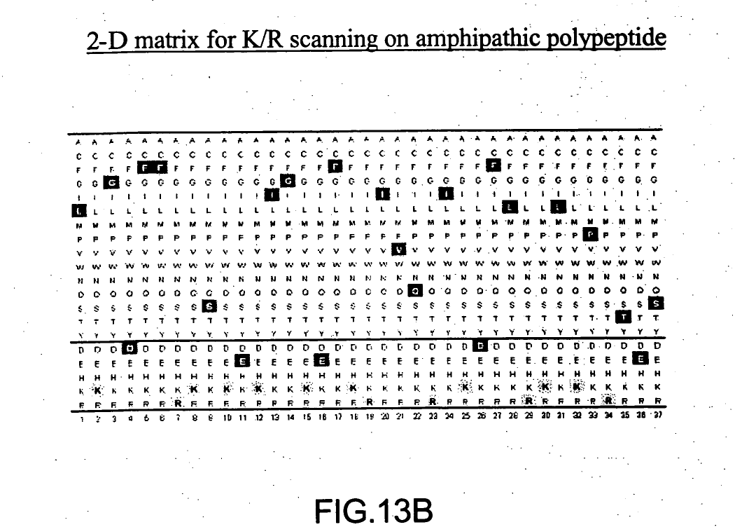

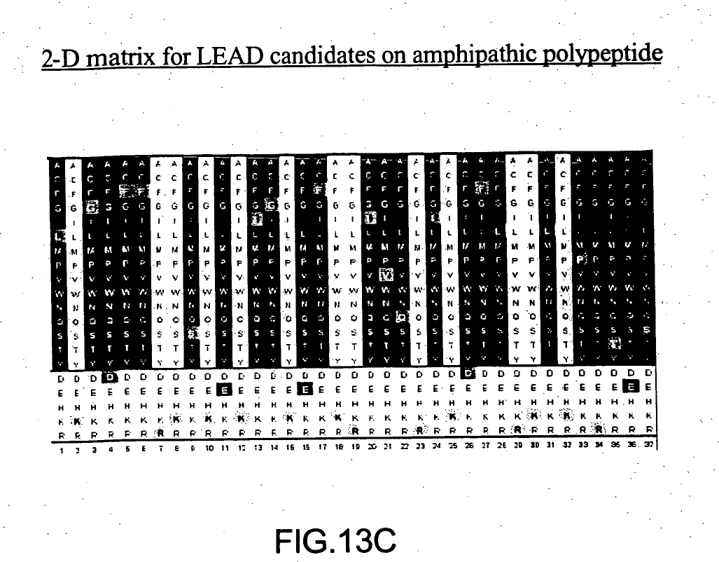

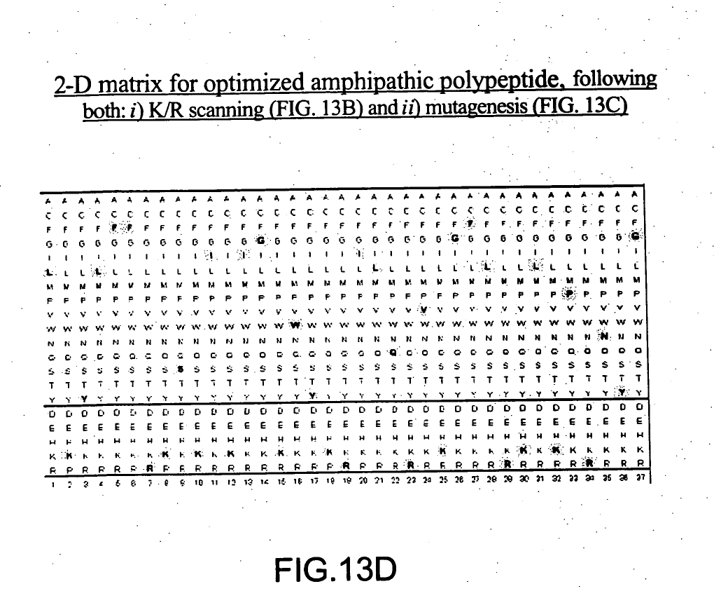

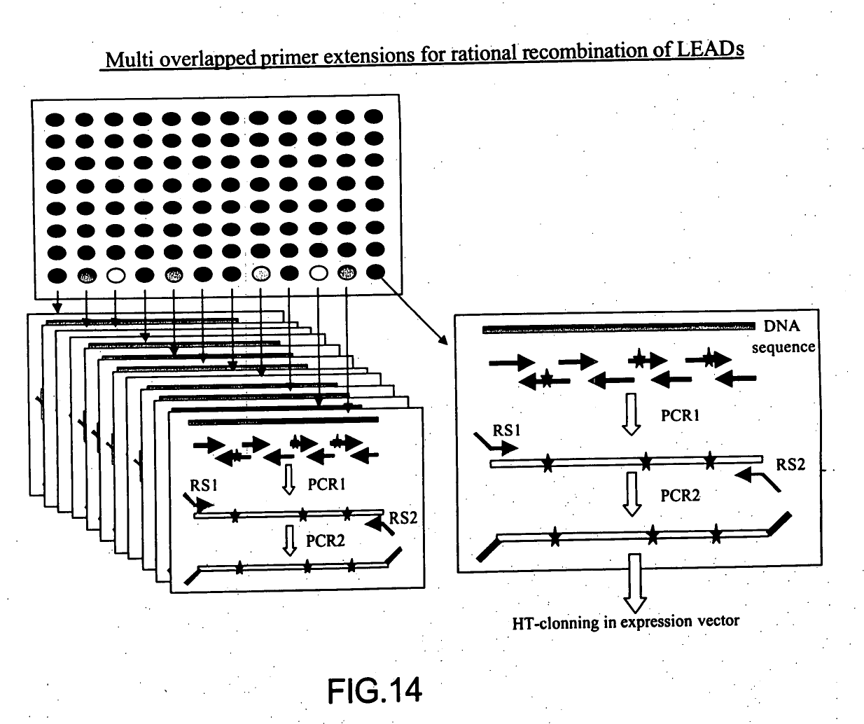

Another method for generation of super leads is multi-overlapped primier extensions. This is a method for the rational evolution of proteins using oligonucleotide-mediated mutagenesis. This method is particularly useful for the rational combination of mutant LEADs to form super-LEADs (see FIG. 14). This method allows the simultaneous introduction of several mutations throughout a small protein or protein-region of known sequence (see, e.g., FIGS. 13A through D). Overlapping oligonucleotides of typically around 70 bases in length (since longer oligonucleotides LEAD to increased error) are designed from the DNA sequence (gene) encoding the mutant LEAD proteins in such a way that they overlap with each other on a region of typically around 20 bases. These overlapping oligonucleotides (including or not point mutations) act as both template and primers in a first step of PCR (using a proofreading polymerase, e.g., Pfu DNA polymerase, to avoid unexpected mutations) to create small amounts of full-length gene. The full-length gene resulting from the first PCR then is selectively amplified in a second step of PCR using flanking primers, each one tagged with a restriction site in order to facilitate subsequent cloning. One multi-overlapped extension process yields a full-length (multi-mutated) nucleic acid molecule encoding a candidate super-LEADs protein having multiple mutations therein derived from LEAD mutant proteins.

Although typically about 70 bases are used to create the overlapping oligonucleotides, the length of additional overlapping oligonucleotides for use herein can range from about 30 bases up to about 100 bases, from about 40 bases up to about 90 bases, from about 50 bases up to about 80 bases, from about 60 bases up to about 75 bases, and from about 65 bases up to about 75 bases. As set forth above, typically about 70 bases are used herein.

Likewise, although typically the overlapping region of the overlapping oligonucleotides is about 20 bases, the length of other overlapping regions for use herein can range from about 5 bases up to about 40 bases, from about 10 bases up to about 35 bases, from about 15 bases up to about 35 bases, from about 15 bases up to about 25 bases, from about 16 bases up to about 24 bases, from about 17 bases up to about 23 bases, from about 18 bases up to about 22 bases, and from about 19 bases up to about 21 bases. As set forth above, typically about 20 bases are used herein for the overlapping region.

E. Exemplary Biological Activities for Alteration by the 2D-Scanning Methods

The 2D methods provided herein are used to alter activity or physical or chemical property of a target polypeptide. Any characteristic (physical, chemical property or activity) can be modified. The protein is selected and the property identified. A suitable assay or method for identifying proteins with the characterisitic.

1. 2-Dimensional Scanning of Proteins for Increased Resistance to Proteolysis

The methods of 2-D scanning permit preparation of proteins modified for a selected trait, activity or other phenotype. Among modifications of interest for therapeutic proteins are those that increase protection against protease digestion while maintaining the requisite biological activity. Such changes are useful for producing longer-lasting therapeutic proteins.

The delivery of stable peptide and protein drugs to patients is a major challenge for the pharmaceutical industry. These types of drugs in the human body are constantly eliminated or taken out of circulation by different physiological processes including internalization, glomerular filtration and proteolysis. The latter is often the limiting process affecting the half-life of proteins used as therapeutic agents in per-oral administration and either intravenous or intramuscular injections.

The 2D-scanning process provided herein for protein evolution is used to effectively improve protein resistance to proteases and thus increase protein half-life in vitro and, ultimately in vivo. The methods provided herein for designing and generating highly stable, longer lasting proteins, or proteins having a longer half-life include: i) identifying some or all possible target sites on the protein sequence that are susceptible to digestion by one or more specific proteases (these sites are referred to herein as is-HITs); ii) identifying appropriate replacing amino acids, specific for each is-HIT, such that upon replacement of one or more of the original, such as native, amino acids at that specific is-HIT, they can be expected to increase the is-HIT's resistance to digestion by protease while at the same time, maintaining or improving the requisite biological activity of the protein (these proteins with replaced amino acids are the “candidate LEADs”); iii) systematically introducing the specific replacing amino acids at every specific is-HIT target position to generate a collection of candidate LEADs containing the corresponding mutant candidate LEAD molecules. Mutants are generated, produced and phenotypically characterized one-by-one, such as in addressable arrays, such that each mutant molecule contains initially an amino acid replacement at only one is-HIT site.

In particular embodiments, such as in subsequent rounds, mutant molecules also can be generated that contain one or more amino acids at one or more is-HIT sites that have been replaced by candidate LEAD amino acids. Those mutant proteins carrying one or more mutations at one or more is-HITs, and that display improved protease resistance are called LEADs (one mutation at one is-HIT) and super-LEADs (mutations at more than one is-HIT).

The first step of the process takes into consideration existing knowledge from different domains. Such knowledge includes:

(1) knowledge about the galenic and the delivery environment (tissue, organ or corporal fluid) of the particular therapeutic protein in order to establish a list of proteases more likely to be found in that environment. For example, a therapeutic protein in per-oral application is likely to encounter typical proteases of the luminal gastrointestinal tract. In contrast, if this protein were injected in the blood circulation, serum proteases would be implicated in the proteolysis. Based on the specific list of proteases involved, the complete list of all amino acid sequences that potentially could be targeted by the proteases in the list is determined.

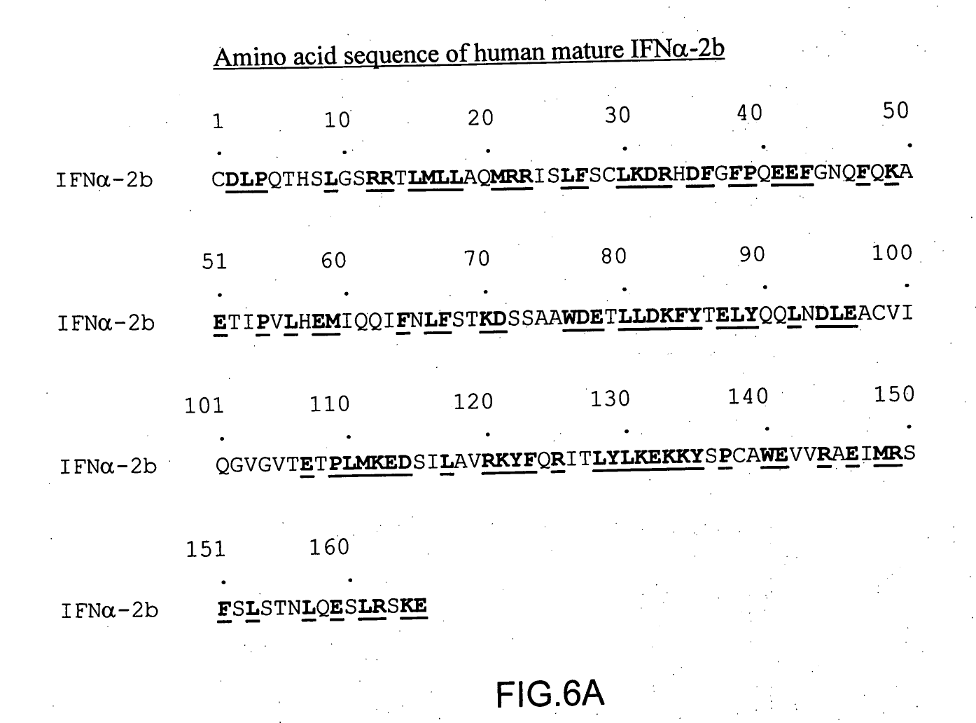

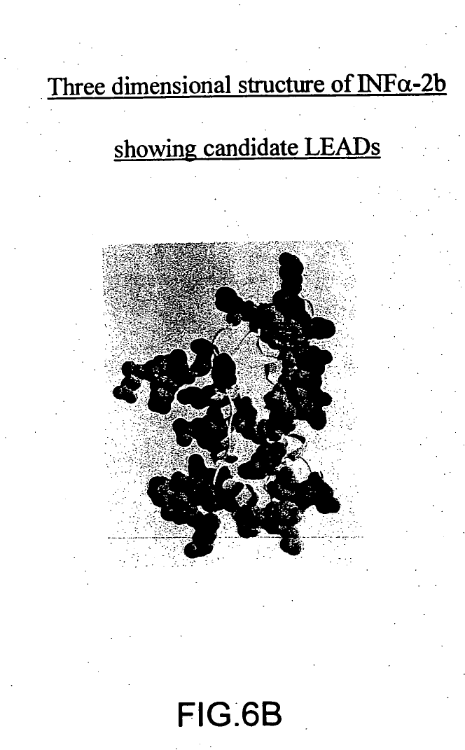

(2) Since protease mixtures in the body are quite complex in composition, almost all the residues in a selected protein sequence potentialy could be targeted for proteolysis (FIG. 6A). Nevertheless, proteins form specific tri-dimensional structures where residues are more or less exposed to the environment and protease action. It can be assumed that those residues constituting the core of a protein are inaccessible to proteases, while those more “exposed” to the environment are better targets for proteases. The probability for every specific amino acid to be “exposed” and accessible to proteases can be taken into account to reduce the number of is-HITs. Consequently, the methods herein consider the analysis with respect to solvent “exposure” or “accessibility” for each individual amino acid in the protein sequence. Solvent accessibility of residues can alternatively be estimated, regardless of any previous knowledge of specific protein structural data, by using an algorithm derived from empirical amino acid probabilities of accessibility, which is expressed in the following equation (Boger et al., Reports of the Sixth International Congress in Immunology, p. 250, 1986):

$$A ( i ) = [ {\prod }_{j = 1}^{6} \_δ {\_}_{i + 4 - j} ] * {[ 0.62 ]}^{- 6}$$

Briefly, these are fractional probabilities (δ_{_{—}}₍ᵢ₎) determined for an amino acid (i) found on the surface of a protein, which are based upon structural data from a set of several proteins. It is thus possible to calculate the solvent accessibility (A) of an amino acid (A(i)) at sequence position (i−2 to i+3, onto a sliding window of length equal to 6) that is within an average surface accessible to solvent of >20 square angstroms (Å²).

The protease accessible target amino acids along the protein sequence, i.e., the amino acids to be replaced, are thus identified and are referred to herein as in silico HITs (is-HITs). Amino acids at the is-HITs are then replaced by residues that render the protein less vulnerable or invulnerable to protease digestion while at the same time maintaining the biological activity of the protein. The choice of the replacing amino acids is complicated by (1) the broad target specificity of certain proteases and (2) the need to preserve the physicochemical properties such as hydrophobicity, charge and polarity, of essential (e.g., catalytic, binding, etc.) residues.

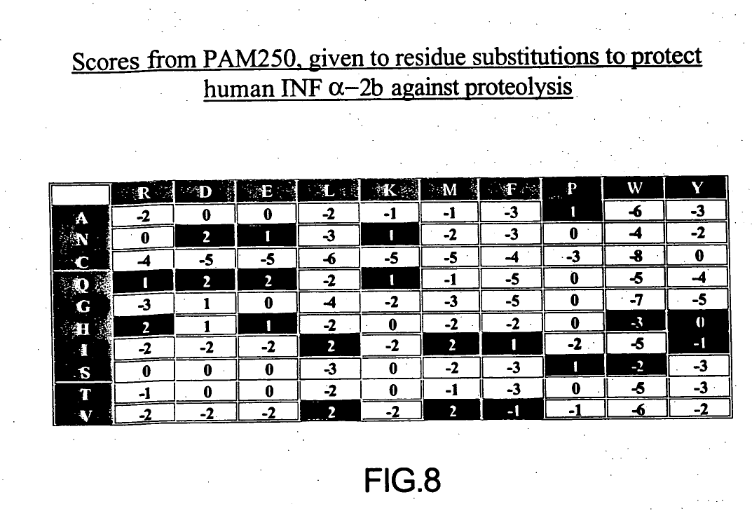

As provided herein, amino acids can be selected by use of the “Percent Accepted Mutation” (PAM; (Dayhoff et al., Atlas of Protein Sequence and Structure, 5 (3):345-352, 1978), FIGS. 7 and 8). PAM values, originally developed to produce alignments between protein sequences, are available in the form of probability matrices, which reflect an evolutionary distance. Since, in a family of proteins or homologous (related) proteins, identical or similar amino acids (85% similarity) are shared, conservative substitutions for, or “allowed point mutations” of the corresponding amino acid residues can be determined throughout an aligned reference sequence. In this regard, “conservative substitutions” of a residue in a reference sequence are those substitutions that are physically and functionally similar to the corresponding reference residues, e.g., that have a similar size, shape, electric charge, chemical properties, including the ability to form covalent or hydrogen bonds, and other propers. Conservative substitutions can be those that exhibit the highest scores and fulfill the PAM matrix criteria in the form of “accepted point mutations”. By comparing a family of scoring matrices, Dayhoff et al., Atlas of Protein Sequence and Structure, 5 (3):345-352, 1978), found consistently higher score significance when using PAM250 matrix to analyze a variety of proteins, known to be distantly related.

In particular, the PAM250 matrix was selected for use. The PAM250 matrix is used, by learning directly from natural evolution, to find replacing amino acids for the is-HITs to generate conservative mutations without affecting the protein function. By using PAM250, candidate replacing amino acids are identified from related proteins from different organisms.

a. Rational Evolution of IFNα-2b for Increased Resistance to Proteolysis

IFNα-2b is used for a variety of applications. Typically it is used for treatment of type B and C chronic hepatitis. Additional indications include, but are not limited to, melanomas, herpes infections, Kaposi sarcomas and some leukemia and lymphoma cases. Patients receiving interferon are subject to frequent repeat applications of the drug. Since such frequent injections generate uncomfortable physiological as well as undesirable psychological reactions in patients, increasing the half-life of interferons and thus decreasing the necessary frequency of interferon injections, would be extremely useful to the medical community. For example, after injection of native human IFNα-2b injection in mice, as a model system, its presence can be detected in the serum between 3 and 10 hours with a half-life of only around 4 hours. The IFNα-2b completely disappears to undetectable levels by 18-24 hours after injection.

Provided herein are mutant variants of the IFNαα-2b protein that display (a) highly improved stability as assessed by resistance to proteases in vitro and by pharmacokinetics studies in mice and (b) at least comparable biological activity as assessed by antiviral and antiproliferative action compared to the unmodified and wild type native IFNα-2b protein and to at least one pegylated derivative of the wild type native IFNα. As a result, the IFNα-2b mutant proteins provided herein confer a higher half-life and at least comparable antiviral and antiproliferation activity (sufficient for a therapeutic effect) with respect to the native protein and to the pegylated derivatives molecules currently being used for the clinical treatment of hepatitis C infection. Thus, the optimized IFNα-2b protein mutants that possess increased resistance to proteolysis and/or glomerular filtration provided herein would result in a decrease in the frequency of injections needed to maintain a sufficient drug level in serum; which should lead to i) higher comfort and acceptance by patients, ii) lower doses necessary to achieve comparable biological effects, and iii) as a consequence of (ii), an attenuation of the (dose-dependent) secondary effects observed in humans.

In particular embodiments, the half-life of the IFNα-2b mutants provided herein is increased by an amount selected from at least 10%, at least 20%, at least 30%, at least 40%, at least 50%, at least 60%, at least 70%, at least 80%, at least 90%, at least 100%, at least 150%, at least 200%, at least 250%, at least 300%, at least 350%, at least 400%, at least 450%, at least 500% or more, when compared to the half-life of native human IFNα-2b in either human blood, human serum or an in vitro mixture containing one or more proteases.

Two methodologies are provided herein to increase the stability of IFNα-2b by amino acid replacement: i) amino acid replacement that leads to higher resistance to proteases by direct destruction of the protease target residue or sequence, while either maintaining or improving the requisite biological activity (such as, for example, antiviral activity or antiproliferation activity), and/or ii) amino acid replacement that leads to a different pattern of N-glycosylation, thus decreasing both glomerular filtration and sensitivity to proteases, while either improving or maintaining the requisite biological activity (such as, for example, antiviral activity or antiproliferation activity).

The 2D-scanning methods provided herein were used to identify the amino acid changes on IFNα-2b that lead to an increase in stability when challenged either with proteases, human blood lysate or human serum. Increasing protein stability to proteases, human blood lysate or human serum, and/or increasing the molecular size is contemplated herein to provide a longer in vivo half-life for the particular protein molecules, and thus to a reduction in the frequency of necessary injections into patients. The biological activities that have been measured for the IFNα-2b molecules are i) their capacity to inhibit virus replication when added to permissive cells previously infected with the appropriate virus, and ii) their capacity to stimulate cell proliferation when added to the appropriate cells. Prior to the measurement of biological activity, IFNα-2b molecules were challenged with proteases, human blood lysate or human serum during different incubation times. The biological activity measured, corresponds then to the residual biological activity following exposure to the protease-containing mixtures.

As set forth above, provided herein are methods for the development of IFNα-2b molecules that, while maintaining the requisite biological activity intact, have been rendered less susceptible to digestion by blood proteases and therefore display a longer half-life in blood circulation. In this particular example, the method used included the following specific steps as set forth in Example 2:
1) Identifying some or all possible target sites on the protein sequence that are susceptible to digestion by one or more specific proteases (these sites are the is-HITs) and
2) Identifying appropriate replacing amino acids, specific for each is-HIT, such that if used to replace one or more of the original amino acids at that specific is-HIT, they can be expected to increase the is-HIT's resistance to digestion by protease while at the same time, keeping the biological activity of the protein unchanged (these replacing amino acids are the “candidate LEADs”).

As set forth in Example 2, the 3-dimensional structure of IFNα-2b obtained from the NMR structure of IFNα-2a (PDB code 1ITF) was used to select only those residues exposed to solvent from a list of residues along the IFNα-2b sequence which can be recognized as a substrate for different enzymes present in the serum. Residue 1 corresponds to the first residue of the mature peptide IFNα-2b encoded by nucleotides 580-1074 of sequence accession No. J00207, SEQ ID NO:1. Using this approach, the following 42 amino acid target positions were identified as is-HITs on IFNα-2b, which numbering is that of the mature protein (SEQ ID NO:1): L3, P4, R12, R13, M16, R22, K23, F27, L30, K31, R33, E41, K49, E58, K70, E78, K83, Y89, E96, E107, P109, L110, M111, E113, L117, R120, K121, R125, L128, K131, E132, K133, K134, Y135, P137, M148, R149, E159, L161, R162, K164, and E165. Each of these positions was replaced by residues defined as compatible by the substitution matrix PAM250 while at the same time not generating any new substrates for proteases. For these 42 is-HITs, the residue substitutions determined by PAM250 analysis were as follows:

R to H, Q
E to H, Q
K to Q, T
L to V, I
M to I, V
P to A, S
Y to I, H.

1) Modified IFNα-2b Proteins with Single Amino Acid Substitutions (is-HITs)

Accordingly provided herein are mutant IFNα-2b proteins that have increased resistance proteolysis compared to the unmodified, typically wild-type, protein. The mutant IFNα-2b proteins include those selected from among proteins containing more single amino acid replacements in SEQ ID NO:1, corresponding to: L by V at position 3; L by I at position 3; P by S at position 4; P by A at position 4; R by H at position 12; R by Q at position 12; R by H at position 13; R by Q at position 13; M by V at position 16; M by I at position 16; R by H at position 22; R by Q at position 22; R by H at position 23; R by Q at position 23; F by I at position 27; F by V at position 27; L by V at position 30; L by I at position 30; K by Q at position 31; K by T at position 31; R by H at position 33; R by Q at position 33; E by Q at position 41; E by H at position 41; K by Q at position 49; K by T at position 49; E by Q at position 58; E by H at position 58; K by Q at position 70; K by T at position 70; E by Q at position 78; E by H at position 78; K by Q at position 83; K by T at position 83; Y by H at position 89; Y by I at position 89; E by Q at position 96; E by H at position 96; E by Q at position 107; E by H at position 107; P by S at position 109; P by A at position 109; L by V at position 110; L by I at position 110; M by V at position 111; M by I at position 111; E by Q at position 113; E by H at position 113; L by V at position 117; L by I at position 117; R by H at position 120; R by Q at position 120; K by Q at position 121; K by Tat position 121; R by H at position 125; R by Q at position 125; L by V at position 128; L by I at position 128; K by Q at position 131; K by T at position 131; E by Q at position 132; E by H at position 132; K by Q at position 133; K by T at position 133; K by Q at position 134; K by T at position 134; Y by H at position 135; Y by I at position 135; P by S at position 137; P by A at position 137; M by V at position 148; M by I at position 148; R by H at position 149; R by Q at position 149; E by Q at position 159; E by H at position 159; L by V at position 161; L by I at position 161; R by H at position 162; R by Q at position 162; K by Q at position 164; K by T at position 164; E by Q at position 165; and E by H at position 165.

2) LEAD Identification

Next the specific replacing amino acids (candidate LEADs) are systematically introduced at every specific is-HIT position to generate a collection containing the corresponding mutant IFNα-2b DNA molecules, as set forth in Example 2. The mutant DNA molecules were used to produce the corresponding mutant IFNα-2b protein molecules by transformation or transfection into the appropriate cells. These protein mutants were assayed for (i) protection against proteolysis, (ii) and for antiviral and antiproliferation activity in vitro, (iii) pharmacokinetics in mice. Of particular interest are mutations that increase these activities of the IFNα-2b mutant proteins compared to unmodified wild type IFNα-2b protein and to pegylated derivates of the wild type protein. Based on the results obtained from these assays, each individual IFNα-2b variant was assigned a specific activity. Those variant proteins displaying the highest stability and/or resistance to proteolysis were selected as LEADs. The candidate LEADs that possessed at least as much residual antiviral activity following protease treatment as the control, native IFNα-2b, before protease treatment were elected as LEADs. The results are set forth in Table 2 of Example 2. Using this method, the following mutants selected as LEADs are provided herein and correspond to the group of proteins containing one or more single amino acid replacements in SEQ ID NO:1, corresponding to: F by V at position 27; R by H at position 33; E by Q at position 41; E by H at position 41; E by Q at position 58; E by H at position 58; E by Q at position 78; E by H at position 78; Y by H at position 89; E by Q at position 107; E by H at position 107; P by A at position 109; Lby V at position 110; M by V at position 111; E by Q at position 113; E by H at position 113; L by V at position 117; L by I at position 117; K by Q at position 121; K by T at position 121; R by H at position 125; R by Q at position 125; K by Q at position 133; K by T at position 133; and E by Q at position 159; E by H at position 159

Also among these are mutations that can have multiple effects. Among mutations described herein, are mutations that result in an increase of the IFNα-2b activity as assessed by detecting the requisite biological activity.

In another embodiment, IFNα-2b proteins that contain a plurality of mutations based on the LEADs (see Tables in the EXAMPLES, listing the candidate LEADs and LEAD sites), are produced to produce IFNα-2b proteins that have activity that is further optimized. Examples of such proteins are described in the EXAMPLES. Other combinations of mutations can be prepared and tested as described herein to identify other LEADs of interest, particularly those that have further increased IFNα-2b antiviral activity or further increased resistance to proteolysis.

b. Rational Evolution of Interferon # (IFNβ) for Increased Resistance to Proteolysis and/or Increased Conformational Stability

The 2D-scanning method provided herein (as well as a 3D-scanning method (see, copending U.S. application Serial No. 37851-922, filed the same day herewith; and described below) were separately applied to interferon β. Treatment with interferon b (IFNβ) is a well established therapy. Typically it is used for treatment of multiple sclerosis (MS). Patients receiving interferon β are subject to frequent repeat applications of the drug. The instability of IFNβ in the blood stream and under the storage conditions is well known. Hence it would be useful to increasing stability (half-life) of IFNβ in serum and also in vitro would improve it as a drug. Provided herein are mutant variants of the IFNβ protein that display improved stability as assessed by resistance to proteases (thereby possessing increased protein half-life) and at least comparable biological activity as assessed by antiviral or antiproliferation activity compared to the unmodified and wild type native IFNβ protein (SEQ ID NO: 499). The IFNβ mutant proteins provided herein confer a higher half-life and at least comparable biological activity with respect to the native sequence. Thus, the optimized IFNβ protein mutants that possess increased resistance to proteolysis provided herein result in a decrease in the frequency of injections needed to maintain a sufficient drug level in serum, thus leading to, for example: i) higher comfort and acceptance by patients, ii) lower doses necessary to achieve comparable biological effects, and iii) as a consequence of (ii), likely attenuation of any secondary effects.

In particular embodiments, the half-life of each IFNβ mutant provided herein is increased by an amount selected from at least 10%, at least 20%, at least 30%, at least 40%, at least 50%, at least 60%, at least 70%, at least 80%, at least 90%, at least 100%, at least 150%, at least 200%, at least 250%, at least 300%, at least 350%, at least 400%, at least 450%, at least 500% or more, when compared to the half-life of native human IFNβ in either human blood, human serum or an in vitro mixture containing one or more proteases. In other embodiments, the half-life of the IFNβ mutants provided herein is increased by an amount selected from at least 6 times, 7 times, 8 times, 9 times, 10 times, 20 times, 30 times, 40 times, 50 times, 60 times, 70 times, 80 times, 90 times, 100 times, 200 times, 300 times, 400 times, 500 times, 600 times, 700 times, 800 times, 900 times, 1000 times, or more, when compared to the half-life of native human IFNβ in either human blood, human serum or an in vitro mixture containing one or more proteases.

Two approaches were used herein to increase the stability of IFNβ by amino acid replacement: i) Resistance to proteases: amino acid replacement that leads to higher resistance to proteases by direct destruction of the protease target residue or sequence, while either maintaining or improving the requisite biological activity (e.g., antiviral and anti-proliferation activity), and/or ii) Conformational stability: amino acid replacement that leads to an increase in conformational stability (i.e. half-life at room temperature or at 37° C.), while either improving or maintaining the requisite biological activity (e.g., antiviral and anti-proliferation activity).

Two methodologies were used to address the improvements described above: (a) 2D-scanning methods were used to identify amino acid changes that lead to improvement in protease resistance and to improvement in conformational stability, and (b) 3D-scanning, which employs structural homology methods (see, copending U.S. application Ser. No. attorney dkt. no. 37851-922, filed the same day herewith, based upon U.S. provisional application Ser. Nos. 60/457,135 and 60/409,898) methods also were used to identify amino acid changes that lead to improvement in protease resistance.

The 2D-scanning and 3D-scanning methods each were used to identify the amino acid changes on IFNβ that lead to an increase in stability when challenged either with proteases, human blood lysate or human serum. Increasing protein stability to proteases, human blood lysate or human serum is contemplated herein to provide a longer in vivo half-life for the particular protein molecules, and thus a reduction in the frequency of necessary injections into patients. The biological activities that have been measured for the IFNβ molecules are i) their capacity to inhibit virus replication when added to permissive cells previously infected with the appropriate virus, and ii) their capacity to stimulate cell proliferation when added to the appropriate cells. Prior to the measurement of biological activity, IFNβ molecules were challenged with proteases, human blood lysate or human serum during different incubation times. The biological activity measured, corresponds then to the residual biological activity following exposure to the proteolytic mixtures.

As set forth above, provided herein are methods for the development of IFNβ molecules that, while maintaining the requisite biological activity intact, have been rendered less susceptible to digestion by blood proteases and therefore display a longer half-life in blood circulation. In this particular example, the method used included the following specific steps as set forth in the Examples: For the improvement of resistance to proteases, by 2D-scanning, the method included:
1) Identifying some or all possible target sites on the protein sequence that are susceptible to digestion by one or more specific proteases (these sites are the is-HITs); and
2) Identifying appropriate replacing amino acids, specific for each is-HIT, such that if used to replace one or more of the original amino acids at that specific is-HIT, they can be expected to increase the is-HIT's resistance to digestion by protease while at the same time, keeping the biological activity of the protein unchanged (these replacing amino acids are the candidate LEADs).

For the improvement of resistance to proteases, by 3D-scanning (structural homology):
1) Identifying some or all possible target sites (is-HITS) on the protein sequence that display an acceptable degree of structural homology around the amino acid positions mutated in the LEAD molecules previously obtained for IFNα using 2D-scanning, and that are susceptible to digestion by one or more specific proteases; and
2) Identifying appropriate replacing amino acids, specific for each is-HIT, such that if used to replace one or more of the original amino acids at that specific is-HIT, they can be expected to increase the is-HIT's resistance to digestion by protease while at the same time, keeping the biological activity of the protein unchanged (these replacing amino acids are the candidate LEADs).

For the improvement of conformational stability, by 2D-scanning, as provided herein:
1) Identifying some or all possible target sites on the protein sequence that are susceptible to being directly involved in the intramolecular flexibility and conformational change (these sites are the is-HITs); and
2) Identifying appropriate replacing amino acids, specific for each is-HIT, such that if used to replace one or more of the original amino acids at that specific is-HIT, they can be expected to increase the thermal stability of the molecule while at the same time, keeping the biological activity of the protein unchanged (these replacing amino acids are the candidate LEADs).
   Using the 2D-scanning and 3D-scanning methods and the 3-dimensional structure of IFNβ, the following amino acid target positions were identified as is-HITs on IFNβ, which numbering is that of the mature protein (SEQ ID NO: 499):

By 3D-scanning: D by Q at position 39, D by H at position 39, D by G at position 39, E by Q at position 42, E by H at position 42, K by Q at position 45, K by T at position 45, K by S at position 45, K by H at position 45, L by V at position 47, L by I at position 47, L by T at position 47, L by Q at position 47, L by H at position 47, L by A at position 47, K by Q at position 52, K by T at position 52, K by S at position 52, K by H at position 52, F by I at position 67, F by V at position 67, R by H at position 71, R by Q at position 71, D by H at position 73, D by G at position 73, D by Q at position 73, E by Q at position 81, E by H at position 81, E by Q at position 107, E by H at position 107, K by Q at position 108, K by T at position 108, K by S at position 108, K by H at position 108, E by Q at position 109, E by H at position 109, D by Q at position 110, D by H at position 110, D by G at position 110, F by I at position 111, F by V at position 111, R by H at position 113, R by Q at position 113, L by V at position 116, L by I at position 116, L by T at position 116, L by Q at position 116, L by H at position 116, L by A at position 116, L by V at position 120, L by I at position 120, L by T at position 120, L by Q at position 120, L by H at position 120, L by A at position 120, K by Q at position 123, K by T at position 123, K by S at position 123, K by H at position 123, R by H at position 124, R by Q at position 124, R by H at position 128, R by Q at position 128, L by V at position 130, L by I at position 130, L by T at position 130, L by Q at position 130, L by H at position 130, L by A at position 130, K by Q at position 134, K by T at position 134, K by S at position 134, K by H at position 134, K by Q at position 136, K by T at position 136, K by S at position 136, K by H at position 136, E by Q at position 137, E by H at position 137, Y by H at position 163, Y by I at position 1631, R by H at position 165, R by Q at position 165.

By 2D-scanning (see Table below for SEQ ID Nos.): M by V at position 1, M by I at position 1, M by T at position 1, M by Q at position 1, M by A at position 1, L by V at position 5, L by I at position 5, L by T at position 5, L by Q at position 5, L by H at position 5, L by A at position 5, F by I at position 8, F by V at position 8, L by V at position 9, L by I at position 9, L by T at position 9, L by Q at position 9, L by H at position 9, L by A at position 9, R by H at position 11, R by Q at position 11, F by I at position 15, F by V at position 15, K by Q at position 19, K by T at position 19, K by S at position 19, K by H at position 19, W by S at position 22, W by H at position 22, N by H at position 25, N by S at position 25, N by Q at position 25, R by H position 27, R by Q position 27, L by V at position 28, L by I at position 28, L by T at position 28, L by Q at position 28, L by H at position 28, L by A at position 28, E by Q at position 29, E by H at position 29, Y by H at position 30, Y by I at position 30, L by V at position 32, L by I at position 32, L by T at position 32, L by Q at position 32, L by H at position 32, L by A at position 32, K by Q at position 33, K by T at position 33, K by S at position 33, K by H at position 33, R by H at position 35, R by Q at position 35, M by V at position 36, M by I at position 36, M by T at position 36, M by Q at position 36, M by A at position 36, D by Q at position 39, D by H at position 39, D by G at position 39, E by Q at position 42, E by H at position 42, K by Q at position 45, K by T at position 45, K by S at position 45, K by H at position 45, L by V at position 47, L by I at position 47, L by T at position 47, L by, Q at position 47, L by H at position 47, L by A at position 47, K by Q at position 52, K by T at position 52, K by S at position 52, K by H at position 52, F by I at position 67, F by V at position 67, R by H at position 71, R by Q at position 71, D by Q at position 73, D by H at position 73, D by G at position 73, E by Q at position 81, E by H at position 81, E by Q at position 85, E by H at position 85, Y by H at position 92, Y by I at position 92, K by Q at position 99, K by T at position 99, K by S at position 99, K by H at position 99, E by Q at position 103, E by H at position 103, E by Q at position 104, E by H at position 104, K by Q at position 105, K by T at position 105, K by S at position 105, K by H at position 105, E by Q at position 107, E by H at position 107, K by Q at position 108, K by T at position 108, K by S at position 108, K by H at position 108, E by Q at position 109, E by H at position 109, D by Q at position 110, D by H at position 110, D by G at position 110, F by I at position 111, F by V at position 111, R by H at position 113, R by Q at position 113, Lby V at position 116, L by I at position 116, L by T at position 116, L by Q at position 116, L by H at position 116, L by A at position 116, L by V at position 120, L by I at position 120, L by T at position 120, L by Q at position 120, L by H at position 120, L by A at position 120, K by Q at position 123, K by T at position 123, K by S at position 123, K by H at position 123, R by H at position 124, R by Q at position 124, R by H at position 128, R by Q at position 128, L by V at position 130, L by I at position 130, L by T at position 130, L by Q at position 130, L by H at position 130, L by A at position 130, K by Q at position 134, K by T at position 134, K by S at position 134, K by H at position 134, K by Q at position 136, K by T at position 136, K by S at position 136, K by H at position 136, E by Q at position 137, E by H at position 137, Y by H at position 138, Y by I at position 138, R by H at position 152, R by Q at position 152, Y by H at position 155, Y by I at position 155, R by H at position 159, R by Q at position 159, Y by H at position 163, Y by I at position 163, R by H at position 165, R by Q at position 165, M by D at position 1, M by E at position 1, M by K at position 1, M by N at position 1, M by R at position 1, M by S at position 1, L by D at position 5, L by E at position 5, L by K at position 5, L by N at position 5, L by R at position 5, L by S at position 5, L by D at position 6, L by E at position 6, L by K at position 6, L by N at position 6, L by R at position 6, L by S at position 6, L by Q at position 6, L by T at position 6, F by E at position 8, F by K at position 8, F by R at position 8, F by D at position 8, L by D at position 9, L by E at position 9, L by K at position 9, L by N at position 9, L by R at position 9, L by S at position 9, Q by D at position 10, Q by E at position 10, Q by K at position 10, Q by N at position 10, Q by R at position 10, Q by S at position 10, Q by T at position 10, S by D at position 12, S by E at position 12, S by K at position 12, S by R at position 12, S by D at position 13, S by E at position 13, S by K at position 13, S by R at position 13, S by N at position 13, S by Q at position 13, S by T at position 13, N by D at position 14, N by E at position 14, N by K at position 14, N by Q at position 14, N by R at position 14, N by S at position 14, N by T at position 14, F by D at position 15, F by E at position 15, F by K at position 15, F by R at position 15, Q by D at position 16, Q by E at position 16, Q by K at position 16, Q by N at position 16, Q by R at position 16, Q by S at position 16, Q by T at position 16, C by D at position 17, C by E at position 17, C by K at position 17, C by N at position 17, C by Q at position 17, C by R at position 17, C by S at position 17, C by T at position 17, L by N at position 20, L by Q at position 20, L by R at position 20, L by S at position 20, L by T at position 20, L by D at position 20, L by E at position 20, L by K at position 20, W by D at position 22, W by E at position 22, W by K at position 22, W by R at position 22, Q by D at position 23, Q by E at position 23, Q by K at position 23, Q by R at position 23, L by D at position 24, L by E at position 24, L by K at position 24, L by R at position 24, W by D at position 79, W by E at position 79, W by K at position 79, W by R at position 79, N by D at position 80, N by E at position 80, N by K at position 80, N by R at position 80, T by D at position 82, T by E at position 82, T by K at position 82, T by R at position 82, I by D at position 83, I by E at position 83, I by K at position 83, I by R at position 83, I by N at position 83, I by Q at position 83, I by S at position 83, I by T at position 83, N by D at position 86, N by E at position 86, N by K at position 86, N by R at position 86, N by Q at position 86, N by S at position 86, N by T at position 86, L by D at position 87, L by E at position 87, L by K at position 87, L by R at position 87, L by N at position 87, L by Q at position 87, L by S at position 87, L by T at position 87, A by D at position 89, A by E at position 89, A by K at position 89, A by R at position 89, N by D at position 90, N by E at position 90, N by K at position 90, N by Q at position 90, N by R at position 90, N by S at position 90, N by T at position 90, V by D at position 91, V by E at position 91, V by K at position 91, V by N at position 91, V by Q at position 91, V by R at position 91, V by S at position 91, V by T at position 91, Q by D at position 94, Q by E at position 94, Q by Q at position 94, Q by N at position 94, Q by R at position 94, Q by S at position 94, Q by T at position 94, I by D at position 95, I by E at position 95, I by K at position 95, I by N at position 95, I by Q at position 95, I by R at position 95, I by S at position 95, I by T at position 95, H by D at position 97, H by E at position 97, H by K at position 97, H by N at position 97, H by Q at position 97, H by R at position 97, H by S at position 97, H by T at position 97, L by D at position 98, L by E at position 98, L by K at position 98, L by N at position 98, L by Q at position 98, L by R at position 98, L by S at position 98, L by T at position 98, V by D at position 101, V by E at position 101, V by K at position 101, V by N at position 101, V by Q at position 101, V by R at position 101, V by S at position 101, V by T at position 101, M by C at position 1, L by C at position 6, Q by C at position 10, S by C at position 13, Q by C at position 16, L by C at position 17, V by C at position 101, L by C at position 98, H by C at position 97, Q by C at position 94, V by C at position 91, N by C at position 90. The following table summarizes the mutants provided herein that exhibit altered resistance to proteolysis and/or higher conformational stability:

| SEQ ID NO. | Mutant |
|---|---|
| 212 | (M1V) |
| 213 | (M1I) |
| 214 | (M1T) |
| 215 | (M1A) |
| 216 | (L5V) |
| 217 | (L5I) |
| 218 | (L5T) |
| 219 | (L5Q) |
| 220 | (L5H) |
| 221 | (L5A) |
| 222 | (F8I) |
| 223 | (F8V) |
| 224 | (L9V) |
| 225 | (L9I) |
| 226 | (L9T) |
| 227 | (L9Q) |
| 228 | (L9H) |
| 229 | (L9A) |
| 230 | (R11H) |
| 231 | (R11Q) |
| 232 | (F15I) |
| 233 | (F15V) |
| 234 | (K19Q) |
| 235 | (K19T) |
| 236 | (K19S) |
| 237 | (K19H) |
| 238 | (W22S) |
| 239 | (W22H) |
| 240 | (N25H) |
| 241 | (N25S) |
| 242 | (N25Q) |
| 243 | (R27H) |
| 244 | (R27Q) |
| 245 | (L28V) |
| 246 | (L28I) |
| 247 | (L28T) |
| 248 | (L28Q) |
| 249 | (L28H) |
| 250 | (L28A) |
| 251 | (E29Q) |
| 252 | (E29H) |
| 253 | (Y30H) |
| 254 | (Y30I) |
| 255 | (L32V) |
| 256 | (L32I) |
| 257 | (L32T) |
| 258 | (L32Q) |
| 259 | (L32H) |
| 260 | (L32A) |
| 261 | (M1Q) |
| 262 | (K33Q) |
| 263 | (K33T) |
| 264 | (K33S) |
| 265 | (K33H) |
| 266 | (R35H) |
| 267 | (R35Q) |
| 268 | (M36V) |
| 269 | (M36I) |
| 270 | (M36T) |
| 271 | (M36Q) |
| 272 | (M36A) |
| 273 | (E85Q) |
| 274 | (E85H) |
| 275 | (Y92H) |
| 276 | (Y92I) |
| 277 | (K99Q) |
| 278 | (K99T) |
| 279 | (K99S) |
| 280 | (K99H) |
| 281 | (E103Q) |
| 282 | (E103H) |
| 283 | (E104Q) |
| 284 | (E104H) |
| 285 | (K105Q) |
| 286 | (K105T) |
| 287 | (K105S) |
| 288 | (K105H) |
| 289 | (Y138H) |
| 290 | (Y138I) |
| 291 | (R152H) |
| 292 | (R152Q) |
| 293 | (Y155H) |
| 294 | (Y155I) |
| 295 | (R159H) |
| 296 | (R159Q) |
| 297 | (M1D) |
| 298 | (M1E) |
| 299 | (M1K) |
| 300 | (M1N) |
| 301 | (M1R) |
| 302 | (M1S) |
| 303 | (L5D) |
| 304 | (L5E) |
| 305 | (L5K) |
| 306 | (L5R) |
| 307 | (L5N) |
| 308 | (L5S) |
| 309 | (L6D) |
| 310 | (L6E) |
| 311 | (L6K) |
| 312 | (L6N) |
| 313 | (L6Q) |
| 314 | (L6R) |
| 315 | (L6S) |
| 316 | (L6T) |
| 317 | (F8D) |
| 318 | (F8E) |
| 319 | (F8K) |
| 320 | (F8R) |
| 321 | (L9D) |
| 322 | (L9E) |
| 323 | (L9K) |
| 324 | (L9N) |
| 325 | (L9R) |
| 326 | (L9S) |
| 327 | (Q10D) |
| 328 | (Q10E) |
| 329 | (Q10K) |
| 330 | (Q10N) |
| 331 | (Q10R) |
| 332 | (Q10S) |
| 333 | (Q10T) |
| 334 | (S12D) |
| 335 | (S12E) |
| 336 | (S12K) |
| 337 | (S12R) |
| 338 | (S13D) |
| 339 | (S13E) |
| 340 | (S13K) |
| 341 | (S13N) |
| 342 | (S13Q) |
| 343 | (S13R) |
| 344 | (S13T) |
| 345 | (N14D) |
| 346 | (N14E) |
| 347 | (N14K) |
| 348 | (N14Q) |
| 349 | (N14R) |
| 350 | (N14S) |
| 351 | (N14T) |
| 352 | (F15D) |
| 353 | (F15E) |
| 354 | (F15K) |
| 355 | (F15R) |
| 356 | (Q16D) |
| 357 | (Q16E) |
| 358 | (Q16K) |
| 359 | (Q16N) |
| 360 | (Q16R) |
| 361 | (Q16S) |
| 362 | (Q16T) |
| 363 | (C17D) |
| 364 | (C17E) |
| 365 | (C17K) |
| 366 | (C17N) |
| 367 | (C17Q) |
| 368 | (C17R) |
| 369 | (C17S) |
| 370 | (C17T) |
| 371 | (L20N) |
| 372 | (L20Q) |
| 373 | (L20R) |
| 374 | (L20S) |
| 375 | (L20T) |
| 376 | (L20D) |
| 377 | (L20E) |
| 378 | (L20K) |
| 379 | (W22D) |
| 380 | (W22E) |
| 381 | (W22K) |
| 382 | (W22R) |
| 383 | (Q23D) |
| 384 | (Q23E) |
| 385 | (Q23K) |
| 386 | (Q23R) |
| 387 | (L24D) |
| 388 | (L24E) |
| 389 | (L24K) |
| 390 | (L24R) |
| 391 | (G78D) |
| 392 | (G78E) |
| 393 | (G78K) |
| 394 | (G78R) |
| 395 | (W79D) |
| 396 | (W79E) |
| 397 | (W79K) |
| 398 | (W79R) |
| 399 | (N80D) |
| 400 | (N80E) |
| 401 | (N80K) |
| 402 | (N80R) |
| 403 | (T82D) |
| 404 | (T82E) |
| 405 | (T82K) |
| 406 | (T82R) |
| 407 | (I83D) |
| 408 | (I83E) |
| 409 | (I83K) |
| 410 | (I83R) |
| 411 | (I83N) |
| 412 | (I83Q) |
| 413 | (I83S) |
| 414 | (I83T) |
| 415 | (N86D) |
| 416 | (N86E) |
| 417 | (N86K) |
| 418 | (N86R) |
| 419 | (N86Q) |
| 420 | (N86S) |
| 421 | (N86T) |
| 422 | (L87D) |
| 423 | (L87E) |
| 424 | (L87K) |
| 425 | (L87R) |
| 426 | (L87N) |
| 427 | (L87Q) |
| 428 | (L87S) |
| 429 | (L87T) |
| 430 | (A89D) |
| 431 | (A89E) |
| 432 | (A89K) |
| 433 | (A89R) |
| 434 | (N90D) |
| 435 | (N90E) |
| 436 | (N90K) |
| 437 | (N90Q) |
| 438 | (N90R) |
| 439 | (N90S) |
| 440 | (N90T) |
| 441 | (V91D) |
| 442 | (V91E) |
| 443 | (V91K) |
| 444 | (V91N) |
| 445 | (V91Q) |
| 446 | (V91R) |
| 447 | (V91S) |
| 448 | (V91T) |
| 449 | (Q94D) |
| 450 | (Q94E) |
| 451 | (Q94K) |
| 452 | (Q94N) |
| 453 | (Q94R) |
| 545 | (Q94S) |
| 455 | (Q94T) |
| 456 | (I95D) |
| 457 | (I95E) |
| 458 | (I95K) |
| 459 | (I95N) |
| 460 | (I95Q) |
| 461 | (I95R) |
| 462 | (I95S) |
| 463 | (I95T) |
| 464 | (H97D) |
| 465 | (H97E) |
| 466 | (H97K) |
| 467 | (H97N) |
| 468 | (H97Q) |
| 469 | (H97R) |
| 470 | (H97S) |
| 471 | (H97T) |
| 472 | (L98D) |
| 473 | (L98E) |
| 474 | (L98K) |
| 475 | (L98N) |
| 476 | (L98Q) |
| 477 | (L98R) |
| 478 | (L98S) |
| 479 | (L98T) |
| 480 | (V101D) |
| 481 | (V101E) |
| 482 | (V101K) |
| 483 | (V101N) |
| 484 | (V101Q) |
| 485 | (V101R) |
| 486 | (V101S) |
| 487 | (V101T) |
| 488 | (M1C) |
| 489 | (V101C) |
| 490 | (L6C) |
| 491 | (L98C) |
| 492 | (Q10C) |
| 493 | (H97C) |
| 494 | (S13C) |
| 495 | (Q94C) |
| 496 | (Q16C) |
| 497 | (N90C) |
| 498 | (V91C) |

2. 2D-scanning of Proteins for Increased Digestibility

The rational mutagenesis methods provided herein also can be used to evolve proteins that are contained in agronomic consumables, crops or foodstuff, such that these proteins display either decreased or abolished secondary effects (such as toxic or allergenic effects) on the consumer. For example, toxic or allergenic effects are attributable to a lack of (or incomplete) digestion of particular proteins in the gut. Thus, it would be useful to increase digestibility of the proteins concerned, while preserving their biological activity. For this purpose, a similar approach to the methods provided herein for increasing protein stability (e.g., see IFNα-2b mutants herein) can be used. Most allergens are resistant to gastric acid and to digestive proteases (Fuchs et al., Food Technology, 50:83-88, 1996; Astwood et al, Nature Biotechnology, 14:1269-1273, 1996), whereas common plant proteins are not. Since agronomic consumables, crops or foodstuff are typically for oral consumption, proteases of the luminal gastrointestinal tract, such as pepsin, trypsin and chimiotrypsin (Woodley, Crit. Rev. Ther. Drug., 11:61-95, 1994; Bernkop-Schnürch, J. Control. Release, 52:1-16, 1998), are included in the list of proteases by which the evolving protein is rendered digestible.

In silico-HITs for the selected protease mixtures as well as the appropriate replacing amino acids can be identified according to the methods provided herein along a particular protein sequence using the PAM250 matrix analysis in such a way that the introduction of protease-specific target residues does not affect the protein's primary biological function in the agronomic consumable, crop or foodstuff. It has been established that physical stability increases the opportunity for a protein to be absorbed in the body and cause systemic effects such as toxicity or allergenicity (Cockburn, J. Biotechnol., (in press), 2002). Accordingly, the introduction of new and frequent protease-specific is-HIT target residues, even in buried regions of the protein structure, is contemplated herein to increase the protein digestibility by a further rapid luminal protease attack (secreted and membrane-bound proteases), which would transiently yield smaller and less allergenic or toxic peptides in the gastrointestinal tract. These methods provided herein are useful in that they are contemplated to reduce the impact of safety and provide a security perspective for genetically modified food.

Accordingly, methods are provided herein for designing and generating mutant proteins that have decreased stability, have increased digestibility, or a shorter lasting in serum or protease mixtures, or have a short half-life, compared to unmodified and/or wild type protein, wherein the methods comprise a first step of identifying some or all possible target sites on the protein sequence that are susceptible to be easily converted, by mutation, into target sites for one or more specific proteases (these sites are the is-HITs). The second step is identifying the appropriate replacing amino acids, specific for each is-HIT, such that if used to replace one or more of the native amino acids at that specific is-HIT, they can be expected to make the is-HIT susceptible to digestion by particular proteases while at the same time, maintaining or improving the desired biological activity of the protein (these replacing amino acids are referred to as “candidate LEADs”). To identify replacing amino acids, the PAM250 matrix described in Example 2 is used.

Next, the specific replacing amino acids (candidate LEADs) are introduced at every specific is-HIT position so as to generate a collection containing the corresponding mutant molecules. Mutants are generated, produced and phenotypically characterized one-by-one, in addressable arrays, such that each mutant molecule contains initially amino acid replacements at only one is-HIT site. In subsequent rounds mutant molecules also can be generated such that they contain one or more amino acids at one or more is-HIT sites that have been replaced by candidate LEAD amino acids. Those mutant proteins carrying one or more mutations at one or more is-HITs, and that display improved protease sensitivity are called LEADs.

3. 2D-scanning of Proteins for Increased Thermostability to Protect Proteins Against Heat

During evolution proteins have evolved to fit to particular roles in the living cells, which determine a specific environment for protein function. Undoubtedly, proteins with industrial interest are not supposed to resist the extreme environmental conditions present in biotechnological processes such as high temperatures and extreme pH. Provided herein are rational mutagenesis methods for the thermostabilization of proteins, based on the 2D-scanning described above, to develop proteins able to perform native functions at high temperatures. Accordingly, provided herein are methods for designing and generating highly thermostable proteins is provided herein comprising a first step of identifying some or all possible target sites on the protein sequence that are susceptible to become, by mutation, a part of a pair of amino acids that would constitute a link or bridge between two distant parts of the protein structure (these sites are referred to herein as the is-HITs). In this case, is-HITs are all amino acids that are located, on the 3-dimensional structure of the protein, in spatial positions such that they face another amino acid at a certain maximal distance. The two facing amino acids involved are considered to make part of a “stabilizing doublet.” The link can be comprised of H-bonds, +/− charge interactions, disulfide bonds. Links or bridges are expected to stabilize the protein structure by introducing rigidity in it.

Once the is-HITs are identified, the second step comprises identifying the appropriate replacing amino acids, specific for each is-HIT, such that if used to replace one or more of the native amino acids at that specific is-HIT, generate a link or bridge in the protein structure while at the same time, maintaining or improving the requisite biological activity of the protein (these replacing amino acids are dubbed “candidate LEADs”). The rationale behind these two steps is to increase protein stability by the introduction of additional linking structures such as disulfide bonds, salt bridges or hydrogen bonds in proteins at every single position where it is possible.

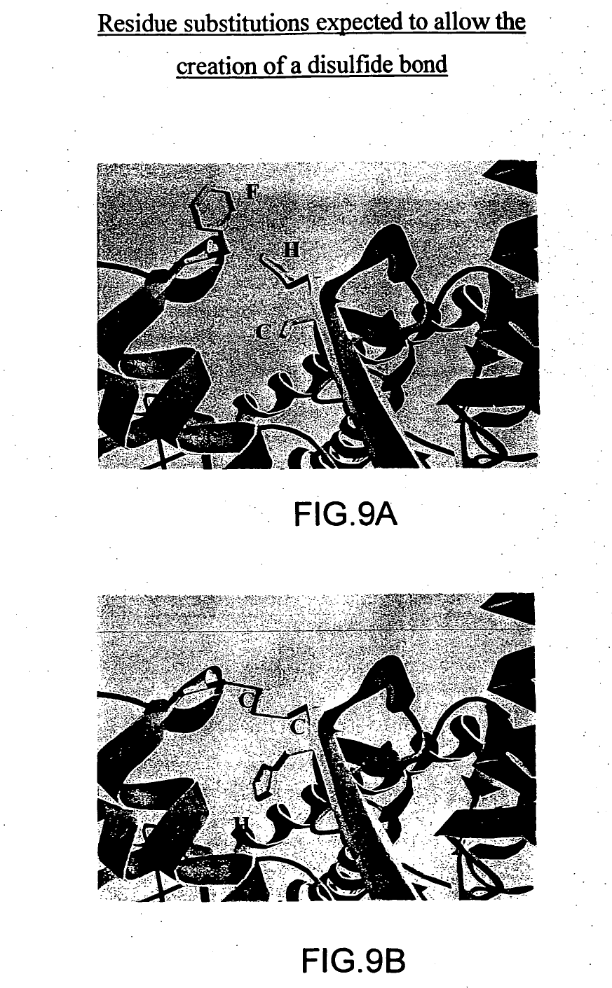

Next, the specific replacing amino acids (candidate LEADs) are introduced at every specific is-HIT position so as to generate a collection containing the corresponding candidate LEAD mutant molecules. Individual mutants are then generated such that, each contains only 2 amino acid replacements, involving a different “stabilizing doublet.” The introduction of additional disulfide bonds includes replacing one or two residues by cysteine along the protein sequence in such a way that their thiol groups remain closer than 2.1 Å, in the tertiary structure of the protein (FIG. 9A through B). The introduction of salt bridges and hydrogen bonds includes replacements of native residues by either charged or polar amino acids, located at the appropriate positions on the protein tertiary structure such that their interaction with each other can generate a tighter structure. In another embodiment, the method to thermostabilize proteins herein includes the replacement of all and every native amino acids located in surface loops of the 3-dimensional structure of the protein, into proline. Again, each initial individual mutant contains only one amino acid replacement at a time. The rationale behind this approach is based on the observation that proline substitutions in amino acid positions involved in loops' are less permissive to flexibility.

Mutants are generated, produced and phenotypically characterized one-by-one, in addressable arrays, such that each mutant molecule contains initially amino acid replacements at only one pair of is-HIT sites. In subsequent rounds mutant molecules also can be generated such that they contain one or more amino acids at one or more pairs of is-HIT sites that have been replaced by candidate LEAD amino acids. Those mutant proteins carrying one or more mutations at one or more is-HITs, and that display improved resistance to heat are called LEADs.

As used herein, the phrase “at high temperatures” refers to at least 5 degrees, at least 10 degrees, at least 15 degrees, at least 20 degrees, at least 25 degrees, at least 30 degrees, at least 40 degrees, at least 50 degrees, at least 60 degrees, at least 70 degrees, at least 80 degrees, at least 90 degrees, up to at least 100 degrees, or more above the optimal temperature for the desired biological activity of the respective native protein. In the above approaches for increasing thermostability, a previous knowledge on the 3-dimensional structure of the protein is necessary. In another rational method to thermostabilize proteins herein, Gly→Ala substitutions are considered regardless the location in the tertiary protein structure and, thus, knowledge of the 3-dimensional structure of the protein is not necessary. The rationale behind this approach is based on the observation that i) glycine is highly permissive to flexibility, and ii) alanine substitutions are considered to be as “entropy-stabilizing” changes. Thus, based on very basic concepts on protein stability, we provide herein a variety of methods to increase protein thermostability. These strategies rely on, but are not limited nor restricted by, predictions and hypotheses on the behavior of specific amino acid replacements.

4. Improvement of Protein Antigenicity

Viral epidemics reflect the effectiveness and remarkable performance of some virus to escape from immune response. Viruses can do this by their amazing ability to mutate and exchange gene segments, leading to a high variability of weakly antigenic sites and/or the lack of production of memory lymphatic cells. Against such infective antigenic drift and antigenic shift (also named reassortment), the body appears defenseless and for some viruses depend on health-assuring vaccination. However, vaccine efficacy also is challenged whenever newly drift variants and/or reassortants emerge. In such cases, new vaccination formulas appear indispensable.

Provided herein are high throughput methods to evolve viral proteins that display low variability and weak immunogenicity, in order to increase both epitope exposure and immunogenicity in an attempt to develop long-lasting efficiency vaccines. A long-lasting vaccine would be composed by viral proteins that have been evolved such that they would expose poorly uncovered epitopes, which could be recognized by antibodies leading thereby to the production of memory lymphocytes. The rationale behind the increase in epitope exposure and immunogenicity would be the local destabilization of the protein structure, intended to expose poorly uncovered epitopes.

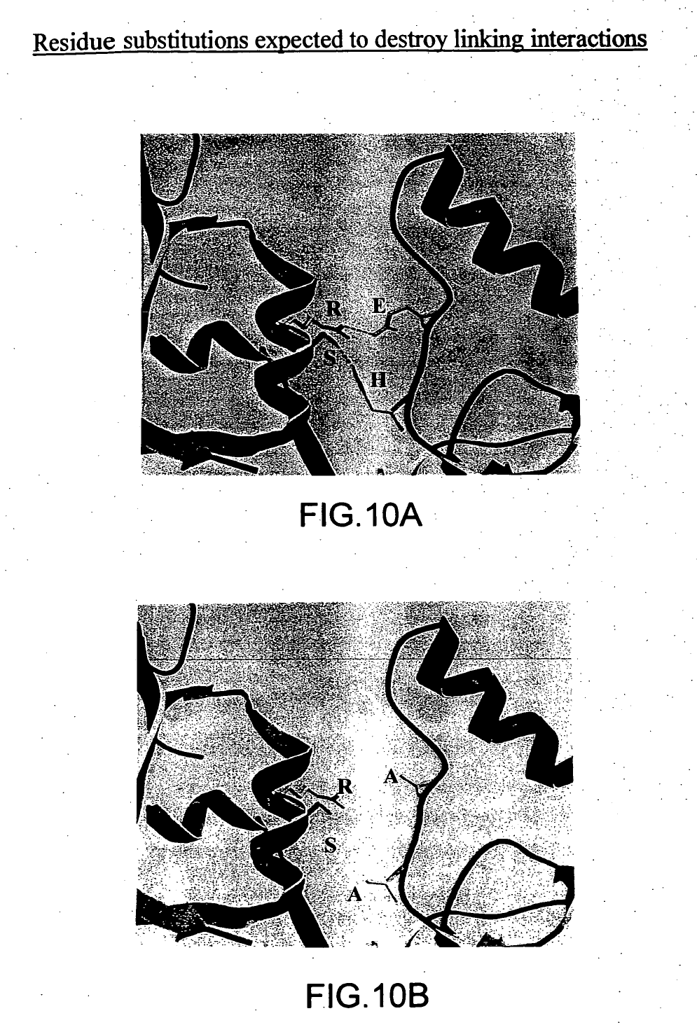

Methods to locally destabilize structural regions of the evolving proteins include herein the use of the basic concepts defining protein stability. In one embodiment, the methods include the substitution of Pro into Ala: the substitution of “(loop)-stabilizing” proline residues, at each position occupied by proline (is-HITs), by the replacing alanine amino acid. These sorts of mutations are expected to decrease rigidity at the level of proline-produced turns, resulting in loops that increase their “mobility” thereby uncovering new epitopes. In another embodiment, the methods include the substitution of Gly into large side chains and high steric hindrance amino acids (F, W, and Y). These replacements are contemplated herein to disturb Gly-compatible turns and thereby lead to the exposure of new epitopes. In another embodiment, a full length Proline-scan is conducted, which is a systematic replacement of native amino acids by proline, along entire length of the protein. The rationale is based on the reported ability of prolines to induce turns in loop regions and kinks in helices, thus leading to localized loss of protein structure. In another embodiment, the methods include the substitution of Cys into Ser. Removing disulfide bonds by replacing cysteine residues by serine would lead to perturbations in the natural protein folding and stability, which is contemplated to herein to increase epitope exposure and immunogenicity. In another embodiment, the replacement of residues involved in the formation of hydrogen bonds and salt bridges on the protein surface, by for instance hydrophobic amino acids, is expected to interfere with the hydrogen bond formation and lead to a local wobbling of protein regions, which would facilitate the presentation of previously covered epitopes (FIGS. 10A through B).

Accordingly, provided herein are methods for designing and generating highly antigenic proteins comprising a first step of identifying some or all possible target sites (the is-HITS) on the protein sequence that are susceptible to significantly change the protein conformation whenever the native amino acids at those target sites are changed by other specific amino acids, such as Proline, Glycine. The second step is to identify the appropriate replacing amino acids, specific for each is-HIT, such that if used to replace one or more of the native amino acids at that specific is-HIT, they can be expected to expose new epitopes or to increase exposure of already exposed epitopes thus increasing immunogenicity of the protein; (these replacing amino acids are named “candidate LEADs”). To identify replacing amino acids, the PAM250 matrix described in Example 2 can be used.

Next, the specific replacing amino acids (candidate LEADs) are introduced at every specific is-HIT position so as to generate a collection containing the corresponding candidate LEAD mutant molecules. Mutants are generated, produced and phenotypically characterized one-by-one, in addressable arrays, such that each mutant molecule contains initially amino acid replacements at only one is-HIT site. In subsequent rounds mutant molecules also can be generated such that they contain one or more amino acids at one or more is-HIT sites that have been replaced by candidate LEAD amino acids. Those mutant proteins carrying one or more mutations at one or more is-HITs, and that display an improved immunogenicity are called LEADs.

Also provided herein are methods for designing and generating highly antigenic proteins comprising performing a “proline-scan” on a particular protein. A collection of mutants is generated in which each individual mutant contains a single amino acid replacement such that each native amino acid is replaced by a proline. Mutants are generated, produced and phenotypically characterized one-by-one, in addressable arrays, such that each mutant molecule contains initially only one amino acid replacement by proline. In subsequent rounds mutant molecules also can be generated such that they contain one or more amino acid replacements by proline. Those mutant proteins carrying one or more mutations (replacements by proline) and that display an improved immunogenicity are called LEADs.

5. Optimization of Polypeptides whose Function Depends on their Amphipathic Character

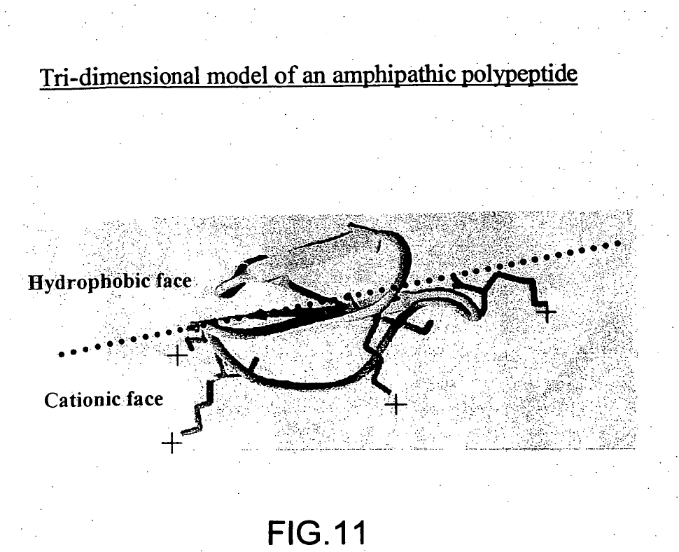

Certain polypeptides are per se amphipathic molecules (i.e., one portion is water-soluble and the other part water-insoluble). Some other polypeptides adopt the amphipathic molecular design depending on the physicochemical conditions of the local environment (including pH, salinity, and temperature) or once a contact with biological membranes is established. For the amphipathic polypeptides or proteins, the amphipathic property is often at the basis of their biological role or activity (FIG. 11). This may involve interactions between protein-protein (glycoprotein, proteins bearing oligosaccharides), protein-substrate, protein-allocrite, protein-ligand, protein-phospholipid, protein-glycolipid, protein-cholesterol or protein-nucleic acid (DNA or RNA). The amphipathic character arises from the presence of hydrophobic and charged (hydrophilic) clusters of amino acids disposed in such a way that two faces can be distinguished in the secondary or tertiary protein structure. In this context, cationic and anionic peptides presenting an amphipathic character are directly concerned. It is contemplated herein that the introduction of specific replacing amino acids bearing a charge that is different from that at the corresponding is-HITs would participate in the formation of new local electrostatic interactions, thus having measurable effects of the protein activity. Such effects can be expected to be highly residue- and/or site-specific. Despite sharing the same electric charge, basic residues, arginine, lysine and histidine, display different chemical properties: arginine and lysine are strongly basic residues (pKa of 12.48 and 10.54 for their respective side chains), whereas histidine is a weakly basic residue (pKa of 6.04 for its side chain).

Methods are provided herein to optimize the biological roles or activities of polypeptides based on their amphipathic character, by performing a “scanning” of charged (i.e., arginine, lysine, histidine, glutamate and aspartate) and/or hydrophobic residues (e.g., valine, leucine, phenylalanine, tryptophan, glycine). Accordingly, depending on the amphipathic polypeptide, one or more of the above replacing residues will follow a sequential replacement of selected residues along the polypeptide sequence, in an attempt to optimize the position, number and nature (cationic or anionic) of charges and hydrophobic residues fitting to an optimized trait. FIGS. 13A through D present steps followed with an exemplary polypeptide, wherein a series of substitutions, after a “K/R scanning” and “hydrophobic scanning,” are intended to optimize its biological role or activity through its amphipathic trait. An innovative method provided herein referred to as “multi-overlapped primer extensions” (see FIG. 14) was used to simultaneously introduce mutations in such short sequences as the one illustrated in FIGS. 13A through D.

Accordingly, provided herein are methods for designing and generating “highly amphipathic” proteins comprising a first step of identifying some or all possible target sites on the protein sequence that are susceptible to significantly change the amphipathic properties of the protein whenever the native amino acids at those sites are changed by other specific amino acids such as arginine or lysine; (these sites are the is-HITs). The next step is identifying the appropriate replacing amino acids, specific for each is-HIT, such that if used to replace one or more of the native amino acids at that specific is-HIT, they can be expected to increase the amphipathic properties of the protein while at the same time, maintaining or improving the requisite biological activity of the protein (these replacing amino acids are referred to as the “candidate LEADs.” To identify replacing amino acids, the PAM250 matrix described in Example 2 can be used.

Next, the specific replacing amino acids (candidate LEADs) are introduced at every specific is-HIT position so that to generate a collection containing the corresponding mutant molecules. Mutants are generated, produced and phenotypically characterized one-by-one, in addressable arrays, such that each mutant molecule contains initially amino acid replacements at only one is-HIT site. In subsequent rounds mutant molecules also can be generated such that they contain one or more amino acids at one or more is-HIT sites that have been replaced by candidate LEAD amino acids. Those mutant proteins carrying one or more mutations at one or more is-HITs, and that display improved amphipathic properties are called LEADs.

Also provided herein are methods for designing and generating highly amphipathic proteins comprising performing either an “arginine-scanning” or a “lysine-scanning” on the particular protein. A collection of mutants is generated in which each individual mutant contains a single amino acid replacement such that each native amino acid is replaced by either arginine or lysine. Mutants are generated, produced and phenotypically characterized one-by-one, in addressable arrays, such that each mutant molecule contains initially only one amino acid replacement by either arginine or lysine. In subsequent rounds mutant molecules also can be generated such that they contain one or more amino acid replacements by either arginine or lysine. Those mutant proteins carrying one or more mutations (replacements by either arginine or lysine) and that display improved amphipathic properties are called LEADs.

6. Ligand-Receptor Interactions

The 2D-scanning methods provided herein also can be used to generate ligand agonists or antagonists (such as negative dominant mutant ligand proteins) for binding to their respective receptors. It is well known that the activity of receptor binding proteins is a direct function of their binding affinity for their respective receptors. For example, strong binding affinity leads to high activity; whereas in contrast, no binding results in the absence of activity. Contemplated herein is the design and generation of: (1) ligand protein mutants with enhanced affinity for their receptors while at the same time having an improved biological activity (agonists); as well as, in contrast, (2) dominant negative ligand protein mutants that bind to their receptors without inducing any cellular response (antagonists).

Accordingly, provided herein are methods for designing and generating high-affinity binding proteins that either maintain (agonists) or have lost (antagonists) their receptor-mediated biological activity while keeping their receptor-binding activity, the method comprising a first step of identifying, in silico, some or all possible target sites on the protein sequence that are susceptible to increase its binding affinity for the corresponding receptor (these sites are the is-HITs). The second step is identifying appropriate replacing amino acids, specific for each is-HIT, such that if used to replace one or more of the native amino acids at that specific is-HIT, they can be expected to increase binding affinity to the corresponding receptor while at the same time, either maintaining the desired biological activity of the protein (agonist protein) or abolishing the biological activity of the (antagonist) protein (these replacing amino acids are referred to as “candidate LEADs”). To identify replacing amino acids, the PAM250 matrix described in Example 2 can be used.

Next, the specific replacing amino acids (candidate LEADs) are introduced at every specific is-HIT position so as to generate a collection containing the corresponding mutant candidate LEAD molecules. Mutants are generated, produced and phenotypically characterized one-by-one, in addressable arrays, such that each mutant molecule contains initially amino acid replacements at only one is-HIT site. In subsequent rounds mutant molecules also can be generated such that they contain one or more amino acids at one or more is-HIT sites that have been replaced by candidate LEAD amino acids.

In another embodiment to generate such antagonist mutants, the first step comprises an amino acid-scanning (e.g., an alanine-scan). The amino acid scanning is used to identify each and every target amino acid residue involved in the binding site(s) on the protein referred to herein as the HITs. This information would then be used, using the 2D-scanning approach and based on the 3-dimensional structure of the protein, to identify the replacing amino acids needed to generate antagonist mutants: The use of “amino acid scanning” to identify the residues involved in the interaction has higher information content than the sole conclusions, which derive from 3-dimensional structure of proteins. While these 3-dimensional protein structures represent conformations that could be non-native and therefore non-active, the amino acid scanning identifies residues at the binding site(s) through a biological assay. Therefore, it reflects conditions that are closer to the physiological conditions than those reflected by 3-dimensional structural methods.

7. Protein Redesign

Provided herein are methods for redesigning and generating new versions of native or modified proteins, such as IFNα-2b (see FIG. 3B). Using these methods, the redesigned protein maintains either sufficient, typically equal or improved levels of a selected phenotype, such as a biological activity, of the original protein, while at the same time its amino acid sequence is changed by replacement of up to less than 1% (i.e., 1, 2, 3 or more amino acid residues), at least 1%, at least 2%, at least 3%, at least 4%, at least 5%, at least 6%, at least 7%, at least 8%, at least 9%, at least 10%, at least 12%, at least 14%, at least 16%, at least 18%, at least 20%, at least 30%, at least 40% up to 50% or more of its native amino acids by the appropriate pseudo-wild type amino acids. Pseudo-wild type amino acids are those amino acids such that when they replace an original, such as native, amino acid at a given position on the protein sequence, the resulting protein displays substantially the same levels of biological activity (or sufficient activity for its therapeutic or other use) compared to the original, such as native, protein. In other embodiments, pseudo-wild type amino acids are those amino acids such that when they replace an original, such as native, amino acid at a given position on the protein sequence, the resulting protein displays the same phenotype, such as levels of biological activity, compared to an original, typically a native, protein. Pseudo-wild type amino acids and the appropriate replacing positions can be detected and identified by any analytical or predictive means; such as for example, by performing an Alanine-scanning. Any other amino acid, particularly another amino acid that has a neutral effect on structure, such as Gly or Ser, also can be used for the scan. All those replacements of original, such as native, amino acids by Ala that do not lead to the generation of a HIT (a protein that has lost the desired biological activity), have either led to the generation of a LEAD (a protein with increased biological activity); or the replacement by Ala will be a neutral replacement, i.e., the resulting protein will display comparable levels of biological activity compared to the original, such as native, protein. The methods provided herein for protein redesign of proteins, such as IFNα-2b, are intended to design and generate “artificial” (versus naturally existing) proteins, such that they contain sequences of amino acids that differ from the naturally-occurring sequences, but that display biological activities characteristic of the original, such as native, protein. These redesigned proteins (pseudo wild types) can be used to avoid potential side effects that might otherwise exist in other forms of proteins for treatment of disease. Other uses of redesigned proteins provided herein are to establish cross-talk between pathways triggered by different proteins; to facilitate structural biology by generating mutants that can be crystallized while maintaining activity; and to destroy an activity of a protein without changing a second activity or multiple additional activities.

In one embodiment, a method for obtaining redesigned proteins comprises i) identifying some or all possible target sites on the protein sequence that are susceptible to amino acid replacement without losing protein activity (protein activity in a largest sense of the term: enzymatic, binding, hormone, etc.) (These sites are the pseudo-wild type, Ψ-wt sites); ii) identifying appropriate replacing amino acids (Ψ-wt amino acids), specific for each Ψ-wt site, such that if used to replace the native amino acids at that specific Ψ-wt site, they can be expected to generate a protein with comparable biological activity compared to the original, such as native, protein, thus keeping the biological activity of the protein substantially unchanged; iii) systematically introducing the specific Ψ-wt amino acids at every specific Ψ-wt position so as to generate a collection containing the corresponding mutant molecules. Mutants are generated, produced and phenotypically characterized one-by-one, in addressable arrays, such that each mutant molecule contains initially amino acid replacements at only one Ψ-wt site. In subsequent rounds mutant molecules also can be generated such that they contain one or more Ψ-wt amino acids at one or more Ψ-wt sites. Those mutant proteins carrying several mutations at a number of Ψ-wt sites, and that display comparable or improved biological activity are called redesigned proteins or Ψ-wt proteins. In particular embodiments, at least 1%, at least 2%, at least 3%, at least 4%, at least 5%, at least 6%, at least 7%, at least 8%, at least 9%, at least 10%, at least 15%, at least 20%, at least 25%, or more of the amino acid residue positions on a particular protein, such as IFNα-2b are replaced with an appropriate pseudo-wild type amino acid.

The first step is an amino acid scan over the full length of the protein. At this step, each and every one of the amino acids in the protein sequence is replaced by a selected reference amino acid, such as alanine. This permits the identification of “redesign-HIT” positions, i.e., positions that are sensitive to amino acid replacement. All of the other positions that are not redesign-HIT positions (i.e., those at which the replacement of the original, such as native, amino acid by the replacing amino acid, for example Ala, does not lead to a drop in protein fitness or biological activity) are referred to herein as “pseudo-wild type” positions. When the replacing amino acid, for example Ala, replaces the original, such as native, amino acid at a non-HIT position, then the replacement is neutral, in terms of protein activity, and the replacing amino acid is said to be a pseudo-wild type amino acid at that position. Pseudo-wild type positions appear to be less sensitive than redesign-HIT positions since they tolerate the amino acid replacement without affecting the protein activity that is being either maintained or improved. Amino acid replacement at the pseudo-wild type positions, result in a non-change in the protein fitness (e.g., possess substantially the same biological activity), while at the same time to a divergence in the resulting protein sequence compared to the original, such as native, sequence.

In one embodiment, to first identify those amino acid positions on the IFNα-2b protein that are involved or not involved in IFNα-2b protein activity, such as binding activity of IFNα-2b to its receptor, an Ala-scan was performed on the IFNα-2b sequence as set forth in Example 4. For this purpose, each amino acid in the IFNα-2b protein sequence was individually changed to Alanine. Any other amino acid, particularly another amino acid that has a neutral effect on structure, such as Gly or Ser, also can be used. Each resulting mutant IFNα-2b protein was then expressed and the activity of the interferon molecule was then assayed. These particular amino acid positions, referred to herein as HITs would in principle not be suitable targets for amino acid replacement to increase protein stability, because of their involvement in the recognition of IFN-receptor or in the downstream pathways involved in IFN activity. For the Ala-scanning, the biological activity measured for the IFNα-2b molecules was: i) their capacity to inhibit virus replication when added to permissive cells previously infected with the appropriate virus and, ii) their capacity to stimulate cell proliferation when added to the appropriate cells. The relative activity of each individual mutant compared to the native protein was assayed. HITs are those mutants that produce a decrease in the activity of the protein (in the example: all the mutants with activities below about 30% of the native activity.

In addition, the Alanine-scan was used to identify the amino acid residues on IFNα-2b that when replaced with alanine correspond to ‘pseudo-wild type’ activity, i.e., those that can be replaced by alanine without leading to a decrease in biological activity. Knowledge of these amino acids is useful for the re-design of the IFNα-2b protein. The results are set forth in Table 5, and include pseudo-wild type amino acid positions of IFNα-2b corresponding to SEQ ID NO:1, amino acid residues: 9, 10, 17, 20, 24, 25, 35, 37, 41, 52, 54, 56, 57, 58, 60, 63, 64, 65, 76, 89, and 90.

Accordingly, provided herein are IFNα-2b mutant proteins that contain one or more pseudo-wild type mutations at amino acid positions of IFNα-2b corresponding to SEQ ID NO:1, amino acid residues: 9, 10, 17, 20, 24, 25, 35, 37, 41, 52, 54, 56, 57, 58, 60, 63, 64, 65, 76, 89, and 90. The mutations can be either one or more of insertions, deletions and/or replacements of the native amino acid residue(s). In one embodiment, the psuedo-wild type replacements are mutations with alanine at each position. In another embodiment, the pseudo-wild type replacements are one or more mutations in SEQ ID NO:1 corresponding to:
P by A at position 4, Q by A at position 5,
T by A at position 6, L by A at position 9,
LG by A at position 10, L by A at position 17,
Q by A at position 20, I by A at position 24,
S by A at position 25, D by A at position 35,
G by A at position 37, G by A at position 39,
E by A at position 41, E by A at position 42,
E by A at position 51, T by A at position 52,
P by A at position 54, V by A at position 55,
L by A at position 56, H by A at position 57,
E by A at position 58, I by A at position 60,
I by A at position 63, F by A at position 64,
N by A at position 65, W by A at position 76,
D by A at position 77, E by A at position 78,
L by A at position 81, Y by A at position 85,
Y by A at position 89, Q by A at position 90,
G by A at position 104, L by A at position 110,
S by A at position 115 and E by A at position 146.

In addition, the IFNα-2b alanine scan revealed the following redesign-HITs having decreased antiviral activity at amino acid positions of IFNα-2b corresponding to SEQ ID NO:1, amino acid residues: 2, 7, 8, 11, 13, 15, 16, 23, 26, 28, 29, 30, 31, 32, 33, 53, 69, 91, 93, 98, and 101. Accordingly, in particular embodiments where it is desired to decrease the viral activity of IFNα-2b, either one or more of insertions, deletions and/or replacements of the native amino acid residue(s) can be carried out at one or more of amino acid positions of IFNα-2b corresponding to SEQ ID NO:1, amino acid residues: 2, 7, 8, 11, 13, 15, 16, 23, 26, 28, 29, 30, 31, 32, 33, 53, 69, 91, 93, 98, and 101.

Each of the redesign mutations set forth above can be combined with one or more of the IFNα-2b candidate LEAD mutations or one or more of the IFNα-2b LEAD mutants provided herein.

F. 3D-Scanning and its Use for Modifying Cytokines

Also provided herein is a method of structural homology analysis for comparing proteins regardless of their underlying amino acid sequences. For a subset of proteins families, such as the family of human cytokines, this information is rationally exploited to produce modified proteins. This method of structural homology analysis can be applied to proteins that are evolved by any method, including the 2D scanning method described herein. When used with the 2D method in which a particular phenotype, activity or characteristic of a protein is modified by 2D analysis, the method is referred to as 3D-scanning.

The use of “structural homology” analysis in combination with the directed evolution methods provided herein provides a powerful technique for identifying and producing various new protein mutants, such as cytokines, having desired biological activities, such as increased resistance to proteolysis. For example, the analysis of the “structural homology” between an optimized mutant version of a given protein and “structurally homologous” proteins allows identification of the corresponding structurally related or structurally similar amino acid positions (also referred to herein as “structurally homologous loci”) on other proteins. This permits identification of mutant versions of the latter that have a desired optimized feature(s) (biological activity, phenotype) in a simple, rapid and predictive manner (regardless of amino acid sequence and sequence homology). Once a mutant version of a protein is developed, then, by applying the rules of structural homology, the corresponding structurally related amino acid positions (and replacing amino acids) on other “structurally homologous” proteins readily are identified, thus allowing a rapid and predictive discovery of the appropriate mutant versions for the new proteins.

3-dimensionally structurally equivalent or similar amino acid positions that are located on two or more different protein sequences that share a certain degree of structural homology, have comparable functional tasks (activities and phenotypes). These two amino acids that occupy substantially equivalent 3-dimensional structural space within their respective proteins then can be said to be “structurally similar” or “structurally related” to each other, even if their precise positions on the amino acid sequences, when these sequences are aligned, do not match with each other. The two amino acids also are said to occupy “structurally homologous loci.” “Structural homology” does not take into account the underlying amino acid sequence and solely compares 3-dimensional structures of proteins. Thus, two proteins can be said to have some degree of structural homology whenever they share conformational regions or domains showing comparable structures or shapes with 3-dimensional overlapping in space. Two proteins can be said to have a higher degree of structural homology whenever they share a higher amount of conformational regions or domains showing comparable structures or shapes with 3-dimensional overlapping in space. Amino acids positions on one or more proteins that are “structurally homologous” can be relatively far way from each other in the protein sequences, when these sequences are aligned following the rules of primary sequence homology. Thus, when two or more protein backbones are determined to be structurally homologous, the amino acid residues that are coincident upon three-dimensional structural superposition are referred to as “structurally similar” or “structurally related” amino acid residues in structurally homologous proteins (also referred to as “structurally homologous loci”). Structurally similar amino acid residues are located in substantially equivalent spatial positions in structurally homologous proteins.

For example, for proteins of average size (approximately 180 residues), two structures with a similar fold will usually display rms deviations not exceeding 3 to 4 angstroms. For example, structurally similar or structurally related amino acid residues can have backbone positions less than 3.5, 3.0, 2.5, 2.0, 1.7 or 1.5 angstrom from each other upon protein superposition. RMS deviation calculations and protein superposition can be carried out using any of a number of methods known in the art. For example, protein superposition and RMS deviation calculations can be carried out using all peptide backbone atoms (e.g., N, C, C(C═O), O and CA (when present)). As another example, protein superposition can be carried out using just one or any combination of peptide backbone atoms, such as, for example, N, C, C(C═O), O and CA (when present). In addition, one skilled in the art will recognize that protein superposition and RMS deviation calculations generally can be performed on only a subset of the entire protein structure. For example, if the protein superposition is carried out using one protein that has many more amino acid residues than another protein, protein superposition can be carried out on the subset (e.g., a domain) of the larger protein that adopts a structure similar to the smaller protein. Similarly, only portions of other proteins can be suitable for superimposition. For example, if the position of the C-terminal residues from two structurally homologous proteins differ significantly, the C-terminal residues can be omitted from the structural superposition or RMS deviation calculations.

Accordingly, provided herein are methods of rational evolution of proteins based on the identification of potential target sites for mutagenesis (is-HITs) through comparison of patterns of protein backbone folding between structurally related proteins, irrespective of the underlying sequences of the compared proteins. Once the structurally related amino acid positions are identified on the new protein, then suitable amino acid replacement criteria, such as PAM analysis, can be employed to identify candidate LEADs for construction and screening as described herein.

For example, analysis of “structural homology” between and among a number of related cytokines was used to identify on various members of the cytokine family, other than interferon alpha, those amino acid positions and residues that are structurally similar or structurally related to those found in the IFNα-2b mutants that have been optimized for improved stability. This method can be applied to any desired phenotype using any protein, such as a cytokine, as the starting material to which an evolution procedure, such as the rational directed evolution procedure of U.S. application Ser. No. 10/022,249 or the 2-dimensional scanning method provided herein, is applied. The structurally corresponding residues are then altered on members of the family to produce additional cytokines with similar phenotypic alterations.

1) Homology

Typically, homology between proteins is compared at the level of their amino acid sequences, based on the percent or level of coincidence of individual amino acids, amino acid per amino acid, when sequences are aligned starting from a reference, generally the residue encoded by the start codon. For example, two proteins are said to be “homologous” or to bear some degree of homology whenever their respective amino acid sequences show a certain degree of matching upon alignment comparison. Comparative molecular biology is primarily based on this approach. From the degree of homology or coincidence between amino acid sequences, conclusions can be made on the evolutionary distance between or among two or more protein sequences and biological systems.

The concept of “convergent evolution” is applied to describe the phenomena by which phylogenetically unrelated organisms or biological systems have evolved to share features related to their anatomy, physiology and structure as a response to common forces, constraints, and evolutionary demands from the surrounding environment and living organisms. Alternatively, “divergent evolution,” is applied to describe the phenomena by which strongly phylogenetically related organisms or biological systems have evolved to diverge from identity or similarity as a response to divergent forces, constraints, and evolutionary demands from the surrounding environment and living organisms.

In the typical traditional analysis of homologous proteins there are two conceptual biases corresponding to: i) “convergent evolution,” and ii) “divergent evolution.” Whenever the aligned amino acid sequences of two proteins do not match well with each other, these proteins are considered “not related” or “less related” with each other and have different phylogenetic origins. There is no (or low) homology between these proteins and their respective genes are not homologous (or show little homology). If these two “non-homologous” proteins under study share some common functional features (e.g., interaction with other specific molecules, activity), they are determined to have arisen by “convergent evolution,” i.e., by evolution of their non-homologous amino acid sequences, in such a way that they end up generating functionally “related” structures.

On the other hand, whenever the aligned amino acid sequences of two proteins do match with each other to a certain degree, these proteins are considered to be “related” and to share a common phylogenetic origin. A given degree of homology is assigned between these two proteins and their respective genes likewise share a corresponding degree of homology. During the evolution of their initial highly homologous amino acid sequence, enough changes can be accumulated in such a way that they end up generating “less-related” sequences and less related function. The divergence from perfect matching between these two “homologous” proteins under study is said come from “divergent evolution.”

2) 3D-Scanning (Structural Homology) Methods

Structural homology refers to homology between the topology and three-dimensional structure of two proteins. Structural homology is not necessarily related to “convergent evolution” or to “divergent evolution,” nor is it related to the underlying amino acid sequence. Rather, structural homology is likely driven (through natural evolution) by the need of a protein to fit specific conformational demands imposed by its environment. Particular structurally homologous “spots” or “loci” would not be allowed to structurally diverge from the original structure, even when its own underlying sequence does diverge. This structural homology is exploited herein to identify loci for mutation.

Within the amino acid sequence of a protein resides the appropriate biochemical and structural signals to achieve a specific spatial folding in either an independent or a chaperon-assisted manner. Indeed, this specific spatial folding ultimately determines protein traits and activity. Proteins interact with other proteins and molecules in general through their specific topologies and spatial conformations. In principle, these interactions are not based solely on the precise amino acid sequence underlying the involved topology or conformation. If protein traits, activity (behavior and phenotypes) and interactions rely on protein topology and conformation, then evolutionary forces and constraints acting on proteins can be expected to act on topology and conformation. Proteins sharing similar functions will share comparable characteristics in their topology and conformation, despite the underlying amino acid sequences that create those topologies and conformations.

Numerous methods are known in the art for identifying structurally related amino acid positions with 3-dimensionally structurally homologous proteins. Exemplary methods include, but are not limited to: CATH (Class, Architecture, Topology and Homologous superfamily) which is a hierarchical classification of protein domain structures based on four different levels (Orengo et al., Structure, 5 (8):1093-1108, 1997); CE (Combinatorial Extension of the optimal path), which is a method that calculates pairwise structure alignments (Shindyalov et al., Protein Engineering, 11 (9):739-747, 1998); FSSP (Fold classification based on Structure-Structure alignment of Proteins), which is a database based on the complete comparison of all 3-dimensional protein structures that currently reside in the Protein Data Bank (PDB) (Holm et al., Science, 273:595-602, 1996); SCOP (Structural Classification of Proteins), which provides a descriptive database based on the structural and evolutionary relationships between all proteins whose structure is known (Murzin et al., J. Mol. Biol., 247:536-540, 1995); and VAST (Vector Alignment Search Tool), which compares newly determined 3-dimensional protein structure coordinates to those found in the MMDB/PDB database (Gibrat et al., Current Opinion in Structural Biology, 6:377-385, 1995).

In an exemplary embodiment, the step-by-step process including the use of a program referred to as TOP (see, and Lu, G., J. Appl. Cryst., 33:176-189, 2000; publicly available, for example, at bioinfol.mbfys.lu.se/TOP is used for protein structure comparison. This program runs two steps for each protein structure comparison. In the first step topology of secondary structure in the two structures is compared. The program uses two points to represent each secondary structure element (alpha helices or beta strands) then systematically searches all the possible super-positions of these elements in 3-dimensional space (defined as the root mean square deviation—rmsd, the angle between the two lines formed by the two points and the line-line distance). The program searches to determine whether additional secondary structure elements can fit by the same superposition operation. If secondary structures that can fit each other exceed a given number, the program identifies the two structures as similar. The program gives as an output a comparison score called “Structural Diversity” that considers the distance between matched α-carbon atoms and the number of matched residues. The lower the “Structural Diversity” score, the more the two structures are similar. In various embodiments herein, the Structural Diversity scores range from 0 up to about 67.

G. 2-D Matrix Representation of Amino Acid Sequence of Protein or Peptide

The amino acid sequence of proteins is usually represented as a sequence stream of letters or names, each representing one individual amino acid in the sequence. This type of linear representation is appropriate to make comparisons on amino acid sequence, homology/heterology, make co-linear representation with DNA nucleotides sequences (thus allowing to represent the genetic code from DNA to protein in a co-linear way). The information content and the analytical potential of this type of representation is limited and thus limits the scope and the perspective of the analysis on protein sequence/structure relationships that are based upon this type of linear amino-acid string representation.

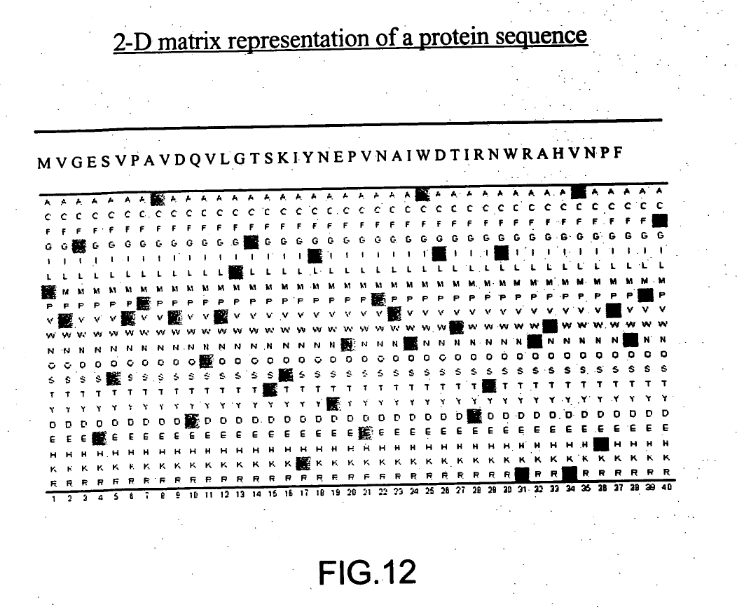

Provided herein is a method of representing the amino acid sequence of a protein (e.g., protein sequencing) that advantageously results in i) higher information content and ii) higher analytical potential, than previous linear amino-acid string sequence representations. These methods for the notation of protein sequence are useful to facilitate the analysis of the relationships between protein sequence and structure, which is currently a bottle-neck for the further development of different fields of biology, including those of directed evolution. The method employs a two-dimensional (2-D) matrix representation of the of protein sequence, where the vertical axis represents the amino acid present at the corresponding position indicated on the horizontal axis. The horizontal axis represents the amino acid position along the length protein sequence (such that the first cell corresponds to amino acid position No. 1, the second cell to amino acid position No. 2, etc.). See FIGS. 12 and 13A through D. The matrix always contains 20 cells in one direction (the amino acid type) and a variable number of position-cells depending on the size of the protein, the number of position-cells equaling the number of amino acids in the protein sequence. In FIG. 12, an exemplary protein sequence is shown above the matrix and within the matrix, such that those cells corresponding to the actual sequence of the protein are indicated with shaded squares.

Once the matrix is constituted, those cells corresponding to the actual sequence of the protein are indicated with either a different color or a sign that differentiates them from the cells not corresponding to the actual protein sequence. For example, for the amino acid sequence: AKRLSL, there will be a sign on the cell corresponding to position No. 1 and amino acid type “A,” a sign for the cell corresponding to position No. 2 and amino acid type “K,” a sign for the cell corresponding to position No. 3 and amino acid type “R,” and so on.

In another embodiment, a 2-D matrix can be employed for representing the nucleotide sequence of a nucleic acid (e.g., nucleic acid sequencing), such as DNA or RNA, whereby the first vertical axis has 4 cells corresponding to nucleotides A, T, G, C; or A, U, G, C, respectively.

### H. EXAMPLES

The following examples are included for illustrative purposes only and are not intended to limit the scope of the invention. The specific methods exemplified can be practiced with other species. The examples are intended to exemplify generic processes.

### Example 1

This example describes a plurality of chronological steps including steps from (i) to (viii):
(i) cloning of IFNα cDNA in a mammalian cell expression plasmid (section A.1)
(ii) generation of a collection of targeted mutants on the IFNα cDNA in the mammalian cell expression plasmid (section B)
(iii) production of IFNα mutants in mammalian cells (section C.1)
(iv) screening and partial in vitro characterization of IFNα mutants produced in mammalian cells in search of lead mutants (section D)
(v) cloning of the lead mutants into a bacterial cell expression plasmid (section A.2)
(vi) expression of lead mutants in bacterial cells (section C.2)
(vii) in vitro characterization of lead mutants produced in bacteria (section D)
(viii) in vivo characterization of lead mutants produced in bacteria (section E).
   A. Cloning of IFNα-2b Encoding cDNA

A.1. Cloning of IFN α-2b cDNA in a Mammalian Cell Expression Plasmid

The IFN α-2b cDNA was first cloned into an mammalian expression vector, prior to the generation of the selected mutations. A library of mutants was then generated such that each individual mutant was created and processed individually, physically separated form each other and in addressable arrays. The mammalian expression vector pSSV9 CMV 0.3 pA was engineered as follows:

The pSSV9 CMV 0.3 pA was cut by PvuII and religated (this step gets rid of the ITR functions), prior to the introduction of a new EcoRI restriction site by Quickchange mutagenesis (Stratagene). The oligonucleotides primers were:

| | |
|---|---|
| EcoRI forward primer | |
| 5′-GCCTGTATGATTTATTGGATGTTGGAATTCC- | (SEQ ID NO:182) |
| CTGATGCGGTATTTTCTCCTTACG-3′ | |
| EcoRI reverse primer | |
| 5′-CGTAAGGAGAAAATACCGCATCAGGGAATT- | (SEQ ID NO:183) |
| CCAACATCCAATAAATCATACAGGC-3′ | |

The construct sequence was confirmed by using the following oligonucleotides:

| | |
|---|---|
| Seq ClaI forward primer: | |
| 5′-CTGATTATCAACCGGGGTACATATGATTGAC-ATGC-3′ | (SEQ ID NO:184) |
| Seq XmnI reverse primer: | |
| 5′-TACGGGATAATACCGCGCCACATAGCAGAA-C-3′ | (SEQ ID NO:185) |

Then, the XmnI-ClaI fragment containing the newly introduced EcoRI site was cloned into pSSV9 CMV 0.3 pA (SSV9 is a clone containing the entire adeno-associated virus (AAV) genome inserted into the PvuII site of plasmid pEMBL (see, Du et al. (1996) Gene Ther 3:254-261)) to replace the corresponding wild-type fragment and produce construct pSSV9-2EcoRI.

The DNA sequence of the IFNα-2b cDNA carried by pDG6 (ATCC accession No. 53169) was confirmed using a pair of internal primers. The sequences of the IFNα-2b-related oligonucleotides for sequencing follow:

| | |
|---|---|
| Seq forward primer | |
| 5′-CCTGATGAAGGAGGACTC-3′ | (SEQ ID NO:186) |
| Seq reverse primer | |
| 5′-CCAAGCAGCAGATGAGTC-3′ | (SEQ ID NO:187) |

Since the beginning of the IFNα-2b encoding cDNA (the signal peptide encoding sequence) is absent in pDG6, it was added using the oligonucleotide (see below) to the amplified gene. First, the IFNα2b cDNA was amplified by PCR using pDG6 as template using the following oligonucleotides as primers:

| | |
|---|---|
| IFNα-2b 5′ primer | |
| 5′-TCAGCTGCAAGTCAAGCTGCTCTGTGGGCTG-3′ | (SEQ ID NO:188) |
| IFNα-2b 3′ primer | |
| 5′-GCTCTAGATCATTCCTTACTTCTTAAACTTTC- | (SEQ ID NO:189) |
| TTGCAAGTTTGTTGAC-3′ | |

The PCR product was then used in an overlapping PCR using the following oligonucleotide sequences, having Hind III or XbaI restriction sites (underlined) or the DNA sequence missing in pDG6 (underlined):

| | |
|---|---|
| IFNα-2b HindIII primer | |
| 5′-CCCAAGCTTATGGCCTTGACCTTTGCTTTACT-GGTG-3′ | (SEQ ID NO:190) |
| IFNα-2b XbaI primer | |
| 5′-GCTCTAGATCATTCCTTACTTCTTAAACTTTC- | (SEQ ID NO:191) |
| TTGCAAGTTTGTTGAC-3′ | |
| IFNα-2b 80bp 5′ primer | |
| 5′-CCCAAGCTTATGGCCTTGACCTTTGCTTTA- | (SEQ ID NO:192) |
| CTGGTGGCCCTCCTGGTGCTCAGCTGCAAGTCAAGCTGCTCTGTGGGCTG-3′ | |

The entire IFNα-2b cDNA was cloned into the pTOPO-TA vector (Invitrogen). After checking gene sequence by automatic DNA sequencing, the HindIII-XbaI fragment containing the gene of interest was subcloned into the corresponding sites of pSSV9-2EcoRI to produce pAAV-EcoRI-IFNalpha-2b (pNB-AAV-IFN alpha-2b).

A.2 Cloning of the IFN α-2b Leads in an E. coli Expression Plasmid

A.2.1 Characterization of the Bacterial Cells

BL21-CodonPlus(DE3)-RP® competent Escherichia coli cells are derived from Stratagene's high-performance BL21-Gold competent cells. These cells enable efficient high-level expression of heterologous proteins in E. coli. Efficient production of heterologous proteins in E. coli is frequently limited by the rarity, in E. coli, of certain tRNAs that are abundant in the organisms from which the heterologous proteins are derived. Availability of tRNAs allows high-level expression of many heterologous recombinant genes in BL21-Codon Plus cells that are poorly expressed in conventional BL21 strains. BL21-CodonPLus(DE3)-RP cells contain a ColE1-compatible, pACYC-based plasmid containing extra copies of the argU and proL tRNA genes.

A.2.2 Cloning of Wild-Type IFN α

To express IFN α-2b in E. coli cDNA encoding the mature form of IFN-2 α-2b was finally cloned into the plasmid pET-11 (Novagen). Briefly, this cDNA fragment was amplified by PCR using the primers SEQ ID Nos. 208 and 209, respectively:

FOR-IFNA-5′ AACATATGTGTGATCTGCCTCAAACCCACAGCCTGGGTAGC 3′
REV-IFNA-5′ AAGGATCCTCATTCCTTACTTCTTAAACTTTCTTGCAAGTTTGTTG3′,

from pSSV9-EcoRI-IFN α-2b (see above), which contains full-length IFN-2 alpha cDNA as a matrix, using Herculase DNA-polymerase (Stratagene). The PCR fragment was subcloned into pTOPO-TA vector (Invitrogen) yielding pTOPO-IFN α-2b. The sequence was verified by sequencing. pET11 IFN α-2b was prepared by insertion of the NdeI-Bam HI (Biolabs) fragment from pTOPO-IFN α-2b into the NdeI-Bam HI sites of pET 11. The DNA sequence of the resulting pET 11-IFN α-2b construct was verified by sequencing and the plasmid was used for IFN α-2b expression in E. coli.

A.2.3 Cloning of IFN α-2b Mutants from the Mammalian Expression Plasmid into the E. coli Expression Plasmid

Lead mutants of Interferon alpha were first generated in the pSSV9-IFNa-EcoRI plasmid. With the only exception of E159H and E159Q, all mutants were amplified using the primers below. Primers contained NdeI (in Forward) and BamHI (in Reverse) restriction sites:

| | |
|---|---|
| FOR-IFNA- | |
| 5′ AAC ATA TGT GTG ATC TGC CTC AAA CCC ACA GCC TGG GTA GC 3′; | SEQ ID No. 210 |
| and | |
| REV-IFNA- | |
| 5′ AAG GAT CCT CAT TCC TTA CTT CTT AAA CTT TCT TGC AAG TTT GTT G 3′. | SEQ ID No. 211 |

Mutants E159H and E159Q were amplified using the following primers on reverse side (primer forward was the same than described above):

| | |
|---|---|
| REV-IFNA-E159H- | |
| 5′ AAG GAT CCT CAT TCC TTA CTT CTT AAA CTG TGT TGC AAG TTT GTT G 3′. | SEQ ID No. 500 |
| REV-IFNA-E159Q- | |
| 5′ AAG GAT CCT CAT TCC TTA CTT CTT AAA CTC TGT TGC AAG TTT GTT G 3′. | SEQ ID No. 501 |

Mutants were amplified with Pfu Turbo Polymerase (Stratagene) according. PCR products were cloned into pTOPO plasmid (Zero Blunt TOPO PCR cloning kit, Invitrogen). The presence of the desired mutations was checked by automatic sequencing. The NdeI+BamHI fragment of the pTOPO-IFNa positive clones was then cloned into NdeI+BamHI sites of the pET11 plasmid.
B. Construction of a Library of IFNα-2b Mutants in a Mammalian Expression Plasmid

A series of mutagenic primers was designed to generate the appropriate site-specific mutations in the IFNα-2b cDNA. Mutagenesis reactions were performed with the Chameleon® mutagenesis kit (Stratagene) using pNB-AAV-IFNα-2b as the template. Each individual mutagenesis reaction was designed to generate one single mutant protein. Each individual mutagenesis reaction contains one and only one mutagenic primer. For each reaction, 25 pmoles of each (phosphorylated) mutagenic primer were mixed with 0.25 pmoles of template, 25 pmoles of selection primer (introducing a new restriction site), and 2 μl of 10× mutagenesis buffer (100 mM Tris-acetate pH 7.5; 100 mM MgOAc; 500 mM KOAc pH 7.5) into each well of 96 well-plates. To allow DNA annealing, PCR plates were incubated at 98° C. during 5 min and immediately placed 5 min on ice, before incubating at room temperature during 30 min. Elongation and ligation reactions were allowed by addition of 7 μl of nucleotide mix (2.86 mM each nucleotide; 1.43× mutagenesis buffer) and 3 μl of a freshly prepared enzyme mixture of dilution buffer (20 mM Tris HCl pH7.5; 10 mM KCl; 10 mM β-mercaptoethanol; 1 mM DTT; 0.1 mM EDTA; 50% glycerol), native T7 DNA polymerase (0.025 U/μl), and T4 DNA ligase (1 U/μl) in a ratio of 1:10, respectively. Reactions were incubated at 37° C. for 1 h before inactivation of T4 DNA ligase at 72° C. during 15 min. In order to eliminate the parental plasmid, 30 μl of a mixture containing 1× enzyme buffer and 10 U of restriction enzyme was added to the mutagenic reactions followed by incubation at 37° C. for at least 3 hours. Next, 90 μl aliquots of XLmutS competent cells (Stratagene) containing 25 mM β-mercaptoethanol were place in ice-chilled deep-well plates. Then, plates were incubated on ice for 10 min with gentle vortex every 2 min. Transformation of competent cells was performed by adding aliquots of the restriction reactions (1/10 of reaction volume) and incubating on ice for 30 min. A heat pulse was performed in a 42° C. water bath for 45 s, followed by incubation on ice for 2 minutes. Preheated SOC medium (0.45 ml) was added to each well and plates were incubated at 37° C. for 1 h with shaking. In order to enrich for mutated plasmids, 1 ml of 2×YT broth medium supplemented with 100 μg/ml ampicillin was added to each transformation mixture followed by overnight incubation at 37° C. with shaking. Plasmid DNA isolation was performed by alkaline lysis using Nucleospin Multi-96 Plus Plasmid Kit (Macherey-Nagel) according to the manufacturer's instructions. Selection of mutated plasmids was performed by digesting 500 μg of plasmid preparation with 10 U of selection endonuclease in an overnight incubation at 37° C. A fraction of the digested reactions (1/10 of the total volume) was transformed into 40 μl of Epicurian coli XL1-Blue competent cells (Stratagene) supplemented with 25 mM β-mercaptoethanol.

Transformation was performed was as described above. Transformants were selected on LB-ampicillin agar plates incubated overnight at 37° C. Isolated colonies were picked up and grown overnight at 37° C. into deep-well plates. Four clones per reaction were screened by endonuclease digestion of a new restriction site introduced by the selection primer. Finally, each mutation that was introduced to produce this library of candidate LEAD IFNα-2b mutant plasmids encoding the proteins set forth in Table 2 of Example 2 was confirmed by automatic DNA sequencing.

C. Production of IFNα-2b Mutants

C.1 In Mammalian Cells

IFNα-2b mutants were produced in 293 human embryo kidney (HEK) cells (obtained from ATCC), using Dubelcco's modified Eagle's medium supplemented with glucose (4.5 g/L; Gibco-BRL) and fetal bovine serum (10%, Hyclone). Cells were transiently transfected with the plasmids encoding the IFNα-2b mutants as follows: 0.6×10⁵ cells were seeded into 6 well-plates and grown for 36 h before transfection Confluent cells at about 70%, were supplemented with 2.5 μg of plasmid (IFNα-2b mutants) and 10 mM poly-ethylene-imine (25 KDa PEI, Sigma-Aldrich). After gently shaking, cells were incubated for 16 h. Then, the culture medium was changed with 1 ml of fresh medium supplemented with 1% of serum. IFNα-2b was measured on culture supernatants obtained 40 h after transfection and stored in aliquots at −80° C. until use.

Supernatants containing IFNα-2b from transfected cells were screened following sequential biological assays as follows. Normalization of IFNα-2b concentration from culture supernatants was performed by enzyme-linked immunoabsorbent assay (ELISA) using a commercial kit (R & D) and following the manufacturer's instructions. This assay includes plates coated with an IFNα-2b monoclonal antibody that can be developed by coupling a secondary antibody conjugated to the horseradish peroxidase (HRP). IFNα-2b concentrations on samples containing (i) wild type IFNα-2b produced under comparable conditions as the mutants, (ii) the IFNα-2b mutants and (iii) control samples(produced from cells expressing GFP) were estimated by using an international reference standard provided by the NIBSC, UK.

C.2 In Bacteria

A volume of 200 ml of culture medium (LB/Ampicillin/Chloramphenicol) was inoculated with 5 ml of pre-culture BL21-pCodon+-pET-IFN α-2b muta overnight at 37° C. with constant shaking (225 rpm). The production of IFN α-2b was induced by the addition of 50 μl of 2M IPTG at DO₆₀₀ₙₘ˜0.6.

The culture was continued for 3 additional hours and was centrifuged at 4° C. and 5000 g for 15 minutes. The supernatant (culture medium) was discarded and bacteria were lysed in 8 ml of lysis buffer by thermal shock (freezing-thawing: 37° C.-15 min; −80° C.-10 min; 37° C.-15 min; −80° C.-10 min; 37° C.-15 min). After centrifugation (10000 g, 15 min, 4° C.), the supernatant (soluble proteins fraction) was discarded, and the precipitated material (insoluble protein fraction containing the IFN α-2b protein as inclusion bodies) was purified.

C.3 Pre-Purification of IFN α-2b as Inclusion Bodies in E. coli

C.3.1 Washing of Inclusion Bodies by Sonication

Pellets containing the inclusion bodies were suspended in 10 ml of buffer and sonicated (80 watts) on ice, 1 second “on”, 1 second “off” for a total of 4 min. Suspensions were then centrifuged (4° C., 10000 g, 15 min), and supernatants were recovered. Pellets were resuspended in 10 ml of buffer for a new sonication/centrifugation cycle. Triton X-100 was then eliminated by two additional cycles of sonication/centrifugation with buffer. Pellets containing the inclusion bodies were recovered and dissolved. The washed supernatants were stored at 4° C.

C.3.2 Solubilization of Inclusion Bodies by Denaturation

Once washed, the inclusion bodies were solubilized in buffer at a concentration estimated in 0.3 mg/ml measuring the OD₂₈₀ (considering the coefficient of molar extinction of IFN α-2b). Solubilization was carried out overnight at 4° C., under shaking.

C.3.3 Renaturation of IFN α-2b by Dialysis of GdnHCl

Samples contained 1 mg of protein at 0.3 mg/ml (5 ml in total) in buffer. The GdnHCl (Hydrochloride Guanidium) present in the samples was eliminated by dialysis (minimum membrane cut=10 kDa) overnight at 4° C. against buffer (1 litre) (final concentration of GdnHCl: 43 Mm). Next, samples were further dialysed against 1 litre of buffer during 2:30 h. This step was repeated two additional times. After dialysis, very little precipitate was visible.

D. Screening and In Vitro Charaterization of IFN α-2b Mutants

Two activities were measured directly on IFN samples: antiviral and antiproliferation activities. Dose (concentration)—response (activity) experiments for antiviral or antiproliferation activity permitted calculation of the ‘potency’ for antiviral and antiproliferation activities, respectively. Antiviral and antiproliferation activities also were measured after incubation with proteolytic samples, such as specific proteases, mixtures of selected proteases, human serum or human blood. Assessment of activity following incubation with proteolytic samples allowed to determine the residual (antiviral or antiproliferation) activity and the respective kinetics of half-life upon exposure to proteases.

D.1. Antiviral Activity

IFNα-2b protects cells against viral infection by a complex mechanism devoted to create an unfavorable environment for viral proliferation. Cellular antiviral response due to IFNα-2b (IFN anti-viral assay) was assessed using an interferon-sensitive HeLa cell line (ATCC accession no. CCL-2) treated with the encephalomyocarditis virus (EMCV). The assessment of either the virus-induced cytopathic effects (CPE) or the amount of EMCV mRNA in extracts of infected cells by RT-PCR was used to determine IFNα activity in samples.

D.1.1 Antiviral Activity—Measure by RT-qPCR

Confluent cells were trypsinized and plated at density 2×10⁴ cells/well in DMEM 5% SVF medium (Day 0). Cells were incubated with IFN α-2b (at a concentration of 500 U/ml) to get 500 pg/ml and 150 pg/well (100 μl of IFN solution), during 24 h at 37° C. prior to be challenged with EMCV (1/1000 dilution; MOI 100). After an incubation of 16 h, when virus-induced CPE was near maximum in untreated cells, the number of EMCV particles in each well was determined by RT-PCR quantification of EMCV mRNA, using lysates of infected cells. RNA from cell extracts was purified after a DNAse/proteinase K treatment (Applied Biosystems). The CPE was evaluated using both Uptibleu (Interchim) and MTS (Promega) methods, which are based on detecting bio-reductions produced by the metabolic activity of cells in a flourometric and colorimetric manner, respectively. In order to produce a standard curve for EMCV quantification, a 22 bp DNA fragment of the capsid protein-cDNA was amplified by PCR and cloned into pTOPO-TA vector (Invitrogen). Next, RT-PCR quantification of known amounts of pTOPO-TA-EMCV capsid gene was performed using the One-step RT-PCR kit (Applied Biosystems) and the following EMCV-related (cloning) oligonucleotides and probe:

| | |
|---|---|
| EMCV forward primer | |
| 5′-CCCCTACATTGAGGCATCCA-3′ | (SEQ ID NO:193) |
| EMCV reverse primer | |
| 5′-CAGGAGCAGGACAAGGTCACT-3′ | (SEQ ID NO:194) |
| EMCV probe | |
| 5′-(FAM)CAGCCGTCAAGACCCAACCGCT(TAMR A)-3′. | (SEQ ID NO:195) |

D.1.2 Antiviral Activity—Measure by CPE

Antiviral activity of IFN α-2b was determined by the capacity of the cytokine to protect Hela cells against EMC (mouse encephalomyocarditis) virus-induced cytopathic effects. The day before, Hela cells (2×10⁵ cells/ml) were seeded in flat-bottomed 96-well plates containing 100 μl/well of Dulbecco's MEM-GlutamaxI-sodium pyruvate medium supplemented with 5% SVF and 0.2% of gentamicin. Cells were growth at 37° C. in an atmosphere of 5% CO₂ for 24 hours.

Two-fold serial dilutions of interferon samples were made with MEM complete media into 96-Deep-Well plates with final concentration ranging from 1600 to 0.6 pg/ml. The medium was aspirated from each well and 100 μl of interferon dilutions were added to Hela cells. Each interferon sample dilution was assessed in triplicate. The two last rows of the plates were filled with 100 μl of medium without interferon dilution samples in order to serve as controls for cells with and without virus.

After 24 hours of growth, a 1/1000 EMC virus dilution solution was placed in each well except for the cell control row. Plates were returned to the CO₂ incubator for 48 hours. Then, the medium was aspirated and the cells were stained for 1 hour with 100 μl of Blue staining solutio to determine the proportion of intact cells. Plates were washed in a distilled water bath. The cell bound dye was extracted using 100 μl of ethylene-glycol mono-ethyl-ether (Sigma). The absorbance of the dye was measured using an Elisa plate reader (Spectramax). The antiviral activity of IFN α-2b samples (expressed as number of IU/mg of proteins) was determined as the concentration needed for 50% protection of the cells against EMC virus-induced cytopathic effects. For proteolysis experiments, each point of for the kinetic measurements was assessed at 500 and 166 pg/ml in triplicate.

D.2 Antiproliferation Activity

Anti-proliferative activity of interferonα-2b was determined by the capacity of the cytokine to inhibit proliferation of Daudi cells. Daudi cells (1×10⁴ cells) were seeded in flat-bottomed 96-well plates containing 50 μl/well of RPMI 1640 medium supplemented with 10% SVF, 1× glutamin and 1 ml of gentamicin. No cell was added to the last row (“H” row) of the flat-bottomed 96-well plates in order to evaluate background absorbance of culture medium.

At the same time, two-fold serial dilutions of interferon samples were made with RPMI 1640 complete media into 96-Deep-Well plates with final concentration ranging from 6000 to 2.9 pg/ml. Interferon dilutions (50 μl) were added to each well containing 50 μl of Daudi cells. The total volume in each well should now be 100 μl. Each interferon sample dilution was assessed in triplicate. Each well of the “G” row of the plates was filled with 50 μl of RPMI 1640 complete media in order to be used as positive control. The plates are incubated for 72 hours at 37° C. in a humidified, 5% CO₂ atmosphere.

After 72 hours of growth, 20 μl of Cell titer 96 Aqueous one solution reagent (Promega) was added to each well and incubated 1H30 at 37° C. in an atmosphere of 5% CO₂. To measure the amount of colored soluble formazan produced by cellular reduction of the MTS, the absorbance of the dye was measured using an Elisa plate reader (spectramax) at 490 nm.

The corrected absorbances (“H” row background value subtracted) obtained at 490 nm were plotted versus concentration of cytokine. The ED50 value was calculated by determining the X-axis value corresponding to one-half the difference between the maximum and minimum absorbance values. (ED50=the concentration of cytokine necessary to give one-half the maximum response).

D.3 Treatment of IFN α-2b with Proteolytic Preparations

Mutants were treated with proteases in order to identify resistant molecules. The resistance of the mutant IFN α-2b molecules compared to wild-type IFN α-2b against enzymatic cleavage (30 min, 25° C.) by a mixture of proteases (containing 1.5 pg of each of the following proteases (1% wt/wt, Sigma): α-chymotrypsin, carboxypeptidase, endoproteinase Arg-C, endoproteinase Asp-N, endoproteinase Glu-C, endoproteinase Lys-C, and trypsin) was determined. At the end of the incubation time, 10 μl of anti-proteases complete, mini EDTA free, Roche (one tablet was dissolved in 10 ml of DMEM and then diluted to 1/1000) was added to each reaction in order to inhibit protease activity. Treated samples were then used to determine residual antiviral or antiproliferation activities.

D.4 Protease Resistance—Kinetic Analysis

The percent of residual IFN α-2b activity over time of exposure to proteases was evaluated by a kinetic study using either (a) 15 pg of chymotrypsin (10% wt/wt), (b) a lysate of human blood at dilution 1/100, (c) 1.5 pg of protease mixture, or (d) human serum. Incubation times were: 0 h, 0.5 h, 1 h, 4 h, 8 h, 16 h, 24 h and 48 h. Briefly, 20 μl of each proteolytic sample (proteases, serum, bnlood) was added to 100 μl of IFN α-2b at 1500 pg/ml (500 U/ml) and incubated for variable times, as indicated. At the appropriate time points, 10 μl of anti-proteases mixture, mini EDTA free, Roche (one tablet was dissolved in 10 ml of DMEM and then diluted to 1/500) was added to each well in order to stop proteolysis reactions. Biological activity assays were then performed as described for each sample in order to determine the residual activity at each time point.

D.5 Performance

The various biological activities, protease resistance and potency of each individual mutant were analyzed using a mathematical model and algorithm (NautScan™; described in French Patent No. 9915884; (published as International PCT application No. WO 01/44809 based on PCT n° PCT/FR00/03503). Data was processed using a Hill equation-based model that uses key feature indicators of the performance of each individual mutant. Mutants were ranked based on the values of their individual performance and those on the top of the ranking list were selected as leads.

E. Pharmacokinetics of Selected Lead Mutants in Mice

IFNα-2b mutants selected on the basis of their overall performance in vitro, were tested for pharmacokinetics in mice in order to have an indication of their half-life in blood in vivo. Mice were treated by subcutaneous (SC) injection with alicuots of each of a number of selected lead mutants. Blood was collected at increasing time points between 0.5 and 48 hs after injection. Inmediatedly after collection, 20 ml of anti-protease solution were added to each blood sample. Serum was obtained for further analysis. Residual IFN-α activity in blood was determined using the tests described in the precedent sections for in vitro characterization. Wild-type IFN α (that had been produced in bacteria under comparable conditions as the lead mutants) as well as a pegylated derivative of IFN α, Pegasys (Roche), also were tested for pharmacokinetics in the same experiments.

### Example 2

This example demonstrates the 2-dimensional (2D) scanning of IFNα-2b for increased resistance to proteolysis.

A) Identifying some or all possible target sites on the protein sequence that are susceptible to digestion by one or more specific proteases (these sites are the is-HITs).

Because IFNα-2b is administered as a therapeutic protein in the blood stream, a set of proteases was identified that were expected to broadly mimic the protease contents in serum. From that list of proteases, a list of the corresponding target amino acids was identified (shown in parenthesis) as follows: α-chymotrypsin (F, L, M, W, and Y), endoproteinase Arg-C(R), endoproteinase Asp-N (D), endoproteinase Glu-C (E), endoproteinase Lys-C (K), and trypsin (K and R) Carboxypeptidase Y, which cleaves non-specifically from the carboxy-terminal ends of proteins, was also included in the protease mixture. The distribution of the target amino acids over the protein sequence spreads over the complete length of the protein, suggesting that the protein is potentially sensitive to protease digestion all over its sequence (FIG. 6A). In order to restrict the number of is-HITs to a lower number of candidate positions, the 3-dimensional structure of the IFNα-2b molecule (PDB code 1RH2) was used to identify and select only those residues exposed on the surface, while discarding from the candidate list those which remain buried in the structure, and therefore stay less susceptible to proteolysis (FIG. 6B).

B) Identifying appropriate replacing amino acids, specific for each is-HIT, such that if used to replace one or more of the original, such as native, amino acids at that specific is-HIT, they can be expected to increase the is-HIT amino acid position's resistance to digestion by protease while at the same time, maintaining or improving the requisite biological activity of the protein (these replacing amino acids are the “candidate LEADs”).

To select the candidate replacing amino acids for each is-HIT position, PAM250 matrix based analysis was used (FIG. 7). In one embodiment, the two highest values in PAM250 matrix, corresponding to the highest occurrence of substitutions between residues (“conservative substitutions” or “accepted point mutations”), were chosen (FIG. 8). Whenever only a conservative substitution was available for a given high value of the PAM250, the following higher value was selected and the totality of conservative substitutions for this value was considered. The replacement of amino acids that are exposed on the surface by cysteine residues (as shown in FIG. 8, while replacing Y by H or I) was explicitly avoided, since this change would potentially lead to the formation of intermolecular disulfide bonds.

Thus, based on the nature of the challenging proteases, and on evolutionary considerations as well as protein structural analysis, a strategy was defined for the rational design of human IFNα-2b mutants having increased resistance to proteolysis which could produce therapeutic proteins having a longer half-life. By using the algorithm PROTEOL (see, e.g., infobiogen.fr), a list of residues along the IFNα-2b sequence was established, which can be recognized as a substrate for different enzymes present in the serum. Because the number of residues in this particular list was high, the 3-dimensional structure of IFNα-2b obtained from the NMR structure of IFNα-2a (PDB code 1ITF) was used to select only those residues exposed to the solvent. Using this approach, 42 positions were identified, which numbering is that of the mature protein (SEQ ID NO:1): L3, P4, R12, R13, M16, R22, K23, F27, L30, K31, R33, E41, K49, E58, K70, E78, K83, Y89, E96, E107, P109, L110, M111, E113, L117, R120, K121, R125, L128, K131, E132, K133, K134, Y135, P137, M148, R149, E159, L161, R162, K164, and E165. Each of these positions was replaced by amino acid residues, such that they are defined as compatible by the substitution matrix PAM250 while at the same time the replacement amino acids do not generate new sites for proteases.

The list of performed residue substitutions as determined by PAM250 analysis is as follows:
R to H, Q
E to H, Q
K to Q, T
L to V, I
M to I, V
P to A, S
Y to I, H

C) Systematically introducing the specific replacing amino acids (candidate LEADs) at every specific is-HIT position to generate a collection containing the corresponding mutant molecules.

The individual IFNα-2b mutants are generated, produced and phenotypically characterized one-by-one, in addressable arrays as set forth in Example 1, such that each mutant molecule contains initially amino acid replacements at only one is-HIT site. LEAD positions were obtained in IFNα-2b variants after a screening for protection against proteases, and comparing protease-untreated and protease-treated variant preparations with the corresponding conditions for the wild-type IFNα-2b. The percent of residual (anti-viral) activity for the IFNα-2b E113H variant after treatment with chymotrypsin, protease mixture, blood lysate or serum was compared to the treated wild-type IFNα-2b. Selected IFNα-2b LEADs are shown in Table 2. A top and side view of IFNα-2b structure in ribbon representation (obtained from NMR structure of IFNα-2b, PDB code 1ITF) depict residues in “space filling” defining (1) the “receptor binding region” as deduced either by “alanine scanning” data and studies by Piehler et al., J. Biol. Chem., 275:40425-40433, 2000, and Roisman et al., Proc. Natl. Acad. Sci USA, 98:13231-13236, 2001, and (2) replacing residues (LEADs) for resistance to proteolysis.

TABLE 2
Selected LEADs of IFNα-2b following protease resistance

| Mutant | SEQ ID No. | Proteolysis protection | IFN antiviral activity |
|---|---|---|---|
| F27V | 83 | Pseudo wt | Pseudo wt |
| R33H | 86 | Pseudo wt | Pseudo wt |
| E41Q | 87 | Increased | Increased |
| E41H | 88 | Pseudo wt | Increased |
| E58Q | 89 | Increased | Pseudo wt |
| E58H | 90 | Increased | Increased |
| E78Q | 92 | Increased | Increased |
| E78H | 93 | Increased | Increased |
| Y89H | 196 | Pseudo wt | Pseudo wt |
| E107Q | 95 | Increased | Pseudo wt |
| E107H | 96 | Increased | Pseudo wt |
| P109A | 97 | Pseudo wt | Pseudo wt |
| L110V | 98 | Pseudo wt | Pseudo wt |
| M111V | 197 | Pseudo wt | Pseudo wt |
| E113H | 101 | Increased | Pseudo wt |
| L117V | 102 | Increased | Pseudo wt |
| L117I | 103 | Increased | Pseudo wt |
| K121Q | 104 | Increased | Pseudo wt |
| R125H | 106 | Increased | Increased |
| R125Q | 107 | Increased | Increased |
| K133Q | 114 | Increased | Increased |
| E159H | 125 | Increased | Pseudo wt |
| E159Q | 124 | Increased | Pseudo wt |

### Example 3

Stabilization of IFNα-2b by Creation of N-Glycosylation Sites

The creation of N-glycosylation sites on the protein was a second strategy that was used to stabilize IFNα-2b Natural human IFNα-2b contains a unique O-glycosylation site at position 129 (the numbering corresponds to the mature protein; SEQ ID NO:1), however, no N-glycosylation sites are found in this sequence. N-glycosylation sites are defined by the N-X-S or N-X-T consensus sequences. Glycosylation has been found to play a role in protein stability. For example, glycosylation has been found to increase bioavailability via higher metabolic stability and reduced clearance. In order to generate more stable IFNα-2b variants, the N-glycosylation consensus sequences indicated above were introduced in the IFNα-2b sequence by mutagenesis. Variants of IFNα-2b carrying new glycosylation sites were assessed as previously described.

The structure of IFNα-2b is characterized by a helix bundle composed of 5 helices (A, B, C, D and E) connected with each other by a series of loops (a large AB loop and three shorter BC, CD, DE loops). The helices are joined together by two disulfide bridges between residues 1/98 and 29/138 of SEQ ID NO:1. The loops are contemplated herein to represent preferential sites for glycosylation given their exposure. Therefore, N-glycosylation sites (N-X-S or N-X-T) were created in each of the loop sequences (Table 3). Selected LEADs and pseudo wild-type IFNα-2b mutants after screening for addition of glycosylation sites are shown in Table 4.

TABLE 3
In silico HITs for addition of glycosylation sites on IFNα-2b

| | SEQ ID | | | |
|---|---|---|---|---|
| Codon No. | No. | N-X-S | SEQ ID No. | N-X-T |
| c2-4 | | D2N/P4S | | D2N/P4T |
| c3-5 | | L3N/Q5S | | L3N/Q5T |
| c4-6 | | P4N/T6S | | P4N/T6T |
| c5-7 | 127 | Q5N/H7S | 128 | Q5N/H7T |
| c6-8 | 129 | T6N/S8S | | T6N/S8T |
| c7-9 | | H7N/L9S | | H7N/L9T |
| c8-10 | 130 | S8N/G10S | 131 | S8N/G10T |
| c9-11 | | L9N/S11S | | L9N/S11T |
| c10-12 | 132 | M21N/R23S | | M21N/R23T |
| c22-24 | | R22N/I24S | | R22N/I24T |
| c23-25 | | R23N/S25S | 133 | R23N/S25T |
| c24-26 | 134 | I24N/L26S | | I24N/L26T |
| c25-27 | 135 | S25N/F27S | 136 | S25N/F27T |
| c26-28 | 137 | L26N/S28S | 138 | L26N/S28T |
| c28-30 | | S28N/L30S | | S28N/L30T |
| c30-32 | 139 | L30N/D32S | | L30N/D32T |
| c31-33 | | K31N/R33S | | K31N/R33T |
| c32-34 | | D32N/H34S | | D32N/H34T |
| c33-35 | 140 | R33N/D35S | 141 | R33N/D35T |
| c34-36 | 142 | H34N/F36S | 143 | H34N/F36T |
| c35-37 | 144 | D35N/G37S | | D35N/G37T |
| c36-38 | 145 | F36N/F38S | 146 | F36N/F38T |
| c37-39 | 147 | G37N/P39S | | G37N/P39T |
| c38-40 | 148 | F38N/Q40S | 149 | F38N/Q40T |
| c39-41 | 150 | P39N/E41S | 151 | P39N/E41T |
| c40-42 | 152 | Q40N/E42S | 153 | Q40N/E42T |
| c41-43 | | E41N/F43S | 155 | E41N/F43T |
| c42-44 | | E42N/G44S | | E42N/G44T |
| c43-45 | | F43N/N45S | | F43N/N45T |
| c44-46 | 156 | G44N/Q46S | 157 | G44N/Q46T |
| c45-47 | 158 | N45N/F47S | 159 | N45N/F47T |
| c46-48 | 160 | Q46N/Q48S | 161 | Q46N/Q48T |
| c47-49 | 162 | F47N/K49S | 163 | F47N/K49T |
| c48-50 | | Q48N/A50S | | Q48N/A50T |
| c49-51 | 164 | K49N/E51S | | K49N/E51T |
| c50-52 | | A50N/T52S | | A50N/T52T |
| c68-70 | | S68N/K70S | | S68N/K70T |
| c70-72 | | K70N/S72S | | K70N/S72T |
| c75-77 | 165 | A75N/D77S | | A75N/D77T |
| c77-79 | | D77N/T79S | | D77N/T79T |
| C100-102 | 166 | I100N/G102S | 167 | I100N/G102T |
| C101-103 | | Q101N/V103S | | Q101N/V103T |
| C102-104 | | G102N/G104S | | G102N/G104T |
| C103-105 | 168 | V103N/V105S | 169 | V103N/V105T |
| C104-106 | | G104N/T106S | 170 | G104N/T106T |
| C105-107 | 171 | V105N/E107S | | V105N/E107T |
| C10--108 | 172 | T106N/T108S | 173 | T106N/T108T |
| C107-109 | 174 | E107N/P109S | 175 | E107N/P109T |
| C108-110 | | T108N/I110S | | T108N/I110T |
| C134-136 | | K134N/S136S | 176 | K134N/S136T |
| C154-156 | | S154N/N156S | | S154N/N156T |
| C155-157 | | T155N/L157S | | T155N/L157T |
| C156-158 | | N156N/Q158S | | N156N/Q158T |
| C157-159 | 177 | L157N/E159S | 178 | L157N/E159T |
| C158-160 | | Q158N/S160S | 179 | Q158N/S160T |
| C159-161 | 180 | E159N/L161S | 181 | E159N/L161T |
| C160-162 | | S160N/R162S | | S160N/R162T |
| C161-163 | | L161N/S163S | | L161N/S163T |
| C162-164 | | R162N/K164S | | R162N/K164T |
| C163-165 | | S163N/E165S | | S163N/E165T |

TABLE 4
Selected LEADs and pseudo wild-type IFNα-2b mutants
after screening for addition of glycosylation sites

| | SEQ ID | | |
|---|---|---|---|
| Mutant | No. | Proteolysis protection | IFN antiviral activity |
| Q5N/H7S | 127 | Increased | Pseudo wt |
| Q5N/H7T | 128 | ND* | ND |
| P39N/E41S | 150 | Increased | Pseudo wt |
| P39N/E41T | 151 | Increased | Pseudo wt |
| Q40N/E42S | 152 | Increased | Pseudo wt |
| Q40N/E42T | 153 | Increased | Pseudo wt |
| E41N/F43S | 154 | Increased | Pseudo wt |
| E41N/F43T | 155 | Increased | Pseudo wt |
| F43N/N45S | | Increased | Pseudo wt |
| F43N/N45T | | ND | ND |
| G44N/Q46S | 156 | ND | ND |
| G44N/Q46T | 157 | Increased | Pseudo wt |
| N45N/F47S | 158 | Increased | Pseudo wt |
| N45N/F47T | 159 | Increased | Pseudo wt |
| Q46N/Q48S | 160 | Increased | Pseudo wt |
| Q46N/Q48T | 161 | ND | ND |
| F47N/K49S | 162 | Increased | Pseudo wt |
| F47N/K49T | 163 | Increased | Pseudo wt |
| I100N/G102S | 166 | Pseudo wt | Increased |
| I100N/G102T | 167 | Pseudo wt | Increased |
| V105N/E107S | 171 | Pseudo wt | Increased |
| V105N/E107T | | Pseudo wt | Increased |
| T106N/T108S | 172 | Pseudo wt | Increased |
| T106N/T108T | 173 | Pseudo wt | Increased |
| E107N/P109S | 174 | Pseudo wt | Increased |
| E107N/P109T | 175 | Pseudo wt | Increased |
| L157N/E159S | 177 | Pseudo wt | Increased |
| L157N/E159T | 178 | Pseudo wt | Increased |
| E159N/L161S | 180 | Pseudo wt | Increased |
| E159N/L161T | 181 | Pseudo wt | Increased |

*ND, not determined

### Example 4

### Redesign of Interferon α-2b Proteins

The use of the protein redesign approach provided herein permits the generation of proteins such that they maintain requisite levels and types of biological activity compared to the native protein while their underlying amino acid sequences have been significantly changed by amino acid replacement. To first identify those amino acid positions on the IFNα-2b protein that are involved or not involved IFNα-2b protein activity, such as binding activity of IFNα-2b to its receptor, an Ala-scan was performed on the IFNα-2b sequence. For this purpose, each amino acid in the IFNα-2b protein sequence was individually changed into Alanine. Any other amino acid, particularly another amino acid that has a neutral effect on structure, such as Gly or Ser, also can be used. Each resulting mutant IFNα-2b protein was then expressed and the antiviral activity of the individual mutants was assayed. The particular amino acid positions that are sensitive to replacement by Ala, referred to herein as HITs would in principle not be suitable targets for amino acid replacement to increase protein stability, because of their involvement in the activity of the molecule. For the Ala-scanning, the biological activity measured for the IFNα-2b molecules was: i) their capacity to inhibit virus replication when added to permissive cells previously infected with the appropriate virus and, ii) their capacity to stimulate cell proliferation when added to the appropriate cells. The relative activity of each individual mutant compared to the native protein was assayed. HITS are those mutants that produce a decrease in the activity of the protein (e.g., in this example, all the mutants with activities below about 30% of the native activity).

In addition, to identify the HIT positions, the Alanine-scan was used to identify the amino acid residues on IFNα-2b that when replaced with alanine lead to a ‘pseudo-wild type’ activity, i.e., those that can be replaced by alanine without leading to a decrease in biological activity.

A collection of mutant molecules was generated and phenotypically characterized such that IFNα-2b proteins with amino acid sequences different from the native ones but that still elicit the same level and type of activity as the native protein were selected. HITs and pseudo wild-type amino acid positions are shown in Table 5.

TABLE 5
HITs and pseudo wild-type positions to IFNα-2b redesign

| Mutants | SEQ ID No. | HITs (viral activity) | Pseudo wt (viral activity) |
|---|---|---|---|
| D2A | 2 | Decreased | |
| P4A | 3 | | Pseudo wt |
| Q5A | 4 | | Pseudo wt |
| T6A | 5 | | Pseudo wt |
| H7A | 6 | Decreased | |
| S8A | 7 | Decreased | |
| L9A | 8 | | Pseudo wt |
| G10A | 9 | | Pseudo wt |
| S11A | 10 | Decreased | |
| R12A | 11 | Decreased | |
| R13A | 12 | Decreased | |
| T14A | 13 | Decreased | |
| L15A | 14 | Decreased | |
| M16A | 15 | Decreased | |
| L17A | 16 | | Pseudo wt |
| Q20A | 17 | | Pseudo wt |
| R23A | 18 | Decreased | |
| I24A | 19 | | Pseudo wt |
| S25A | 20 | | Pseudo wt |
| L26A | 21 | Decreased | |
| S28A | 22 | Decreased | |
| C29A | 23 | Decreased | |
| L30A | 24 | Decreased | |
| K31A | 25 | Decreased | |
| D32A | 26 | Decreased | |
| R33A | 27 | Decreased | |
| D35A | 28 | | Pseudo wt |
| G37A | 29 | | Pseudo wt |
| G39A | 30 | | Pseudo wt |
| E41A | 31 | | Pseudo wt |
| E42 | 32 | | Pseudo wt |
| F43A | 33 | Decreased | |
| N45A | 34 | Decreased | |
| F47A | 35 | Decreased | |
| E51A | 36 | | Pseudo wt |
| T52A | 37 | | Pseudo wt |
| I53A | 38 | Decreased | |
| P54A | 39 | | Pseudo wt |
| V55A | 40 | | Pseudo wt |
| L56A | 41 | | Pseudo wt |
| H57A | 42 | | Pseudo wt |
| E58A | 43 | | Pseudo wt |
| M59A | 44 | Decreased | |
| I60A | 45 | | Pseudo wt |
| I63A | 46 | | Pseudo wt |
| F64A | 47 | | Pseudo wt |
| N65A | 48 | | Pseudo wt |
| L66A | 49 | Decreased | |
| F67A | 50 | Decreased | |
| T69A | 51 | Decreased | |
| K70A | 52 | Decreased | |
| D71A | 53 | Decreased | |
| S72A | 54 | Decreased | |
| W76A | 55 | | Pseudo wt |
| D77A | 56 | | Pseudo wt |
| E78A | 57 | | Pseudo wt |
| L81A | 58 | | Pseudo wt |
| D82A | 59 | Decreased | |
| K83A | 60 | Decreased | |
| F84A | 61 | Decreased | |
| Y85A | 62 | | Pseudo wt |
| Y89A | 63 | | Pseudo wt |
| Q90A | 64 | | Pseudo wt |
| Q91 | 65 | Decreased | |
| N93A | 66 | Decreased | |
| D94A | 67 | Decreased | |
| C98A | 68 | Decreased | |
| V99A | 69 | Decreased | |
| Q101A | 207 | Decreased | |
| G104A | 70 | | Pseudo wt |
| L110A | 71 | | Pseudo wt |
| S115A | 72 | | Pseudo wt |
| Y122A | 73 | Decreased | |
| W140A | 74 | Decreased | |
| E146A | 75 | | Pseudo wt |

### EXAMPLE 5

### Super LEADS of Interferon α-2b Protein by Additive Directional Mutagenesis

The use of an additive directional mutagenesis approach provided a method for the assembly of multiple mutations previously present on the individual LEAD molecules in a single mutant protein thereby generating super-LEAD mutant proteins. In this method, a collection of nucleic acid molecules encoding a library of new mutant molecules is generated, tested and phenotypically characterized one-by-one in addressable arrays. Super-LEAD mutant molecules are such that each molecule contains a variable number and type of LEAD mutations

Using the LEADs obtained in Example 2, six series of mutant molecules were generated with more than one mutation per molecule as shown in Table 6. Some SuperLEAD mutant molecules were phenotypically characterized and the results are shown in Table 7. As shown in the table not all SuperLEADS have improved activity compared with the original Leads; some showed decreased activity of some type.

TABLE 6
Schema of LEADs position for SuperLEADS generation

Series 1
m1 = E41H
m1 + m2 = E41H + Y89H
Series 2
m1 = E58Q
m1 + m2 = E58Q + F27V
Series 3
m1 = R125H
m1 + m2 = R125H + M111V
Series 4
m1 = E159H
m1 + m2 = E159H + Y89H
Series 5
m1 = K121Q
m1 + m2 = K121Q + P109A
m1 + m2 + m3 = K121Q + P109A + K133Q
Series 6
m1 = E78H
m1 + m2 = E78H + R33H
m1 + m2 + m3 = E78H + R33H + E58H
m1 + m2 + m3 + m4 = E78H + R33H + E58H + L11OV

TABLE 7
SuperLEADs of IFNα-2b multiple mutants

| | | Proteolysis | IFN |
|---|---|---|---|
| Mutant | SEQ ID No. | protection | antiviral activity |
| E41H | 88 | Pseudo wt | Increased |
| Y89H | 196 | Pseudo wt | Pseudo wt |
| E41H/Y89H/ | 198 | Increased | Increased |
| N45D** | | | |
| E58Q | 89 | Increased | Pseudo wt |
| F27V | 83 | Pseudo wt | Pseudo wt |
| E58Q/F27V | 200 | Increased | Pseudo wt |
| R125H | 106 | Increased | Increased |
| M111V | 197 | Pseudo wt | Pseudo wt |
| R125H/ | 205 | Increased | Increased |
| M111V | | | |
| E159H | 125 | | |
| Y89H | 196 | | |
| E159H/Y89H | 206 | | |
| K121Q | 104 | Increased | Pseudo wt |
| P109A | 97 | Pseudo wt | Pseudo wt |
| K133Q | 114 | Increased | Increased |
| K121Q/P109A | 202 | Increased | Pseudo wt |
| K121Q/P109A/ | 203 | Increased | Increased |
| K133Q/ | | | |
| G102R** | | | |
| E78H | 93 | Increased | Increased |
| R33H | 86 | Pseudo wt | Pseudo wt |
| E58H | 89 | Increased | Increased |
| L110V | 98 | Pseudo wt | Pseudo wt |
| E78H/R33H/ | 201 | Decreased | Decreased |
| E58H/L110V | | | |

Four mutants with additional mutations to those selected by the rational mutagenesis were generated in the E. coli MutS strain and were detected by sequencing. The mutants were the following: E41Q/D94G; L117V/A139G; E41H/Y89H/N45D; and K121Q/P109A/K133Q/G102R.

### Example 6

### Cloning of IFN β in pNAUT, a Mammalian Cell Expression Plasmid

The cDNA encoding IFN β (see, SEQ ID No. 499) was cloned into a mammalian expression vector, prior to the generation of the selected mutations. A collected of predesigned, targeted mutants was then generated such that each individual mutant was created and processed individually, physically separated form each other and in addressable arrays. The mammalian expression vector pSSV9 CMV 0.3 pA (see, Example 1) was engineered as follows:

The pSSV9 CMV 0.3 pA was cut by PvuII and religated (this step gets rid of the ITR functions), prior to the introduction of a new EcoRI restriction site by Quickchange mutagenesis (Stratagene). The oligonucleotides sequences used, follow:

| | |
|---|---|
| EcoRI forward primer: | |
| 5′-GCCTGTATGATTTATTGGATGTTGGAATTCC- | (SEQ ID NO:182) |
| CTGATGCGGTATTTTCTCCTTACG-3′ | |
| EcoRI reverse prime: | |
| 5′-CGTAAGGAGAAAATACCGCATCAGGGAATT- | (SEQ ID NO:183) |
| CCAACATCCAATAAATCATACAGGC-3′ | |

The construct sequence was confirmed by using the following oligonucleotides:

| | |
|---|---|
| Seq ClaI forward primer: | |
| 5′-CTGATTATCAACCGGGGTACATATGATTGAC-ATGC-3′ | (SEQ ID NO:184) |
| Seq XmnI reverse primer: | |
| 5′-TACGGGATAATACCGCGCCACATAGCAGAA-C-3′. | (SEQ ID NO:185) |

Then, the XmnI-ClaI fragment containing the newly introduced EcoRI site was cloned into pSSV9 CMV 0.3 pA to replace the corresponding wild-type fragment and produce construct pSSV9-2EcoRI.

The IFN β-cDNA was obtained from the pIFNβ1 (ATCC) construct. The sequence of the IFN β-cDNA was confirmed by sequencing using the primers below:

| | |
|---|---|
| Seq forward primer: | |
| 5′-CCTGATGAAGGAGGACTC-3′ | (SEQ ID NO:186) |
| Seq reverse primer: | |
| 5′-CCAAGCAGCAGATGAGTC-3′. | (SEQ ID NO:187) |

The verified IFN β-encoding cDNA first was cloned into the pTOPO-TA vector (Invitrogen). After checking of the cDNA sequence by automatic DNA sequencing, the HindIII-XbaI fragment containing the IFN cDNA was subcloned into the corresponding sites of pSSV9-2EcoRI, leading to the construct pAAV-EcoRI-IFNbeta (pNB-AAV-IFN beta) Finally the fragment Pvu II of plasmid pNB-AAV-IFN beta was subcloned in PvuII site of pUC 18 leading the final construct pUC-CMVIFNbetapA called pNAUT-IFNbeta

Production and Normalization of IFNβ in Mammalian Cells

IFN-β was produced in CHO Chinese Hamster Ovarian cells (obtained from ATCC), using Dubelcco's modified Eagle's medium supplemented with glucose (4.5 g/L; Gibco-BRL) and fetal bovine serum (5%, Hyclone). Cells were transiently transfected as follows: 0.6×10⁵ cells were seeded into 6 well plates and grown for 24 h before transfection. Confluent cells at about 70%, were supplemented with 1.0 μg of plasmid (from the library of IFN β mutants) by lipofectamine plus reagent (Invitrogen). After gently shaking, cells were incubated for 24 h with 1 ml of culture medium supplemented with 1% of serum. IFN-β was obtained from culture supernatants 24 h after transfection and stored in aliquots at −80° C. until use.

Preparations of IFN-β produced from transfected cells were screened following sequential biological assays as follows. Normalization of IFN-β concentration from culture supernatants was performed by ELISA. IFN-β concentrations from wild type, and mutants samples were estimated by using an international reference standard provided by the NIBSC, UK.

Screening and In Vitro Charaterization of IFN-β Mutants

Two activities were measured directly on IFN samples: antiviral and antiproliferation activities. Dose (concentration)—response (activity) experiments for antiviral or antiproliferation activity allowed for the calculation of the ‘potency’ for antiviral and antiproliferation activities, respectively. Antiviral and antiproliferation activities also were measured after incubation with proteolytic samples such as specific proteases, mixtures of selected proteases, human serum or human blood. Assessment of activity following incubation with proteolytic samples allowed to determine the residual (antiviral or antiproliferation) activity and the respective kinetics of half-life upon exposure to proteases.

Antiviral Activity—Measured by Cytopathic Effects (CPE)

Antiviral activity of IFN β was determined by the capacity of the cytokine to protect Hela cells against EMC (mouse encephalomyocarditis) virus-induced cytopathic effects. The day before, Hela cells (2×10⁵ cells/ml) were seeded in flat-bottomed 96-well plates containing 100 μl/well of Dulbecco's MEM-GlutamaxI-sodium pyruvate medium supplemented with 5% SVF and 0.2% of gentamicin. Cells were growth at 37° C. in an atmosphere of 5% CO₂ for 24 hours.

Two-fold serial dilutions of interferon samples were made with MEM complete media into 96-Deep-Well plates with final concentration ranging from 1600 to 0.6 pg/ml. The medium was aspirated from each well and 100 μl of interferon dilutions were added to Hela cells. Each interferon sample dilution was assessed in triplicate. The two last rows of the plates were filled with 100 μl of medium without interferon dilution samples in order to serve as controls for cells with and without virus.

After 24 hours of growth, a 1/1000 EMC virus dilution solution was placed in each well, except for the cell control row. Plates were returned to the CO₂ incubator for 48 hours. Then, the medium was aspirated and the cells were stained for 1 hour with 100 μl of Blue staining solutio to determine the proportion of intact cells. Plates were washed in a distilled water bath. The cell bound dye was extracted using 100 μl of ethylene-glycol mono-ethyl-ether (Sigma). The absorbance of the dye was measured using an Elisa plate reader (Spectramax). The antiviral activity of IFN β samples (expressed as number of IU/mg of proteins) was determined as the concentration needed for 50% protection of the cells against EMC virus-induced cytopathic effects. For proteolysis experiments, each point of the kinetic was assessed at 800 and 400 pg/ml in triplicate.

Anti-Proliferative Activity

Anti-proliferative activity of IFNβ was determined by assessing the capacity of the cytokine to inhibit proliferation of Daudi cells. Daudi cells (1×10⁴ cells) were seeded in flat-bottomed 96-well plates containing 50 μl/well of RPMI 1640 medium supplemented with 10% SVF, 1× glutamine and 1 ml of gentamicin. No cell was added to the last row (“H” row) of the flat-bottomed 96-well plates in order to evaluate background absorbance of culture medium.

At the same time, two-fold serial dilutions of interferon samples were made with RPMI 1640 complete media into 96-Deep-Well plates with final concentration ranging from 6000 to 2.9 pg/ml. Interferon dilutions (50 μl) were added to each well containing 50 μl of Daudi cells. The total volume in each well should now be 100 μl. Each interferon sample dilution was assessed in triplicate. Each well of the “G” row of the plates was filled with 50 μl of RPMI 1640 complete media in order to be used as positive control. The plates were incubated for 72 hours at 37° C. in a humidified, 5% CO₂ atmosphere.

After 72 hours of growth, 20 μl of Cell titer 96 Aqueous one solution reagent (Promega) was added to each well and incubated 1H30 at 37° C. in an atmosphere of 5% CO₂. To measure the amount of colored soluble formazan produced by cellular reduction of the MTS, the absorbance of the dye was measured using an Elisa plate reader (spectramax) at 490 nm.

The corrected absorbances (“H” row background value subtracted) obtained at 490 nm were plotted versus concentration of cytokine. The ED50 value was calculated by determining the X-axis value corresponding to one-half the difference between the maximum and minimum absorbance values. (ED50=the concentration of cytokine necessary to give one-half the maximum response).

Treatment of IFNβ with Proteolytic Preparations

Mutants were treated with proteases in order to identify resistant molecules. The resistance of the mutant IFN βmolecules compared to wild-type IFN β against enzymatic cleavage (120 min, 25° C.) by a mixture of proteases (containing 1.5 pg of each of the following proteases (1% wt/wt, Sigma): α-chymotrypsin, carboxypeptidase, endoproteinase Arg-C, endoproteinase Asp-N, endoproteinase Glu-C, endoproteinase Lys-C, and trypsin) was determined. At the end of the incubation time, 10 μl of anti-proteases complete, mini EDTA free, Roche (one tablet was dissolved in 10 ml of DMEM and then diluted to 1/1000) was added to each reaction in order to inhibit protease activity. Treated samples were then used to determine residual antiviral or antiproliferation activities.

Protease Resistance—Kinetic Analysis

The percent of residual IFN β activity over time of exposure to proteases was evaluated by a kinetic study using 1.5 pg of protease mixture. Incubation times were: 0 h, 0.5 h, 2 h, 4 h, 8 h, 12 h, 24 h and 48 h. Briefly, 20 μl of each proteolytic sample (proteases, serum, bnlood) was added to 100 μl of IFN β at 400 and 800 pg/ml and incubated for variable times, as indicated. At the appropriate time points, 10 μl of anti-proteases mixture, mini EDTA free, Roche (one tablet was dissolved in 10 ml of DMEM and then diluted to 1/500) was added to each well in order to stop proteolysis reactions. Biological activity assays were then performed as described for each sample in order to determine the residual activity at each time point.

Performance

The various biological activities, protease resistance and potency of each individual mutant were analyzed using a mathematical model and algorithm (NautScan™; Fr. Patent No. 9915884; see, also published International PCT application No. WO 01/44809 based on PCT no PCT/FR00/03503). Data was processed using a Hill equation-based model that uses key feature indicators of the performance of each individual mutant. Mutants were ranked based on the values of their individual performance and those on the top of the ranking list were selected as leads.

Using the 2D-scanning and 3D-scanning methods described above in addition to the 3-dimensional structure of IFNβ, the following amino acid target positions were identified as is-HITs on IFNβ, which numbering is that of the mature protein (SEQ ID NO:499):

By 3D-scanning: D by Q at position 39, D by H at position 39, D by G at position 39, E by Q at position 42, E by H at position 42, K by Q at position 45, K by T at position 45, K by S at position 45, K by H at position 45, L by V at position 47, L by I at position 47, L by T at position 47, L by Q at position 47, L by H at position 47, L by A at position 47, K by Q at position 52, K by T at position 52, K by S at position 52, K by H at position 52, F by I at position 67, F by V at position 67, R by H at position 71, R by Q at position 71, D by H at position 73, D by G at position 73, D by Q at position 73, E by Q at position 81, E by H at position 81, E by Q at position 107, E by H at position 107, K by Q at position 108, K by T at position 108, K by S at position 108, K by H at position 108, E by Q at position 109, E by H at position 109, D by Q at position 110, D by H at position 110, D by G at position 110, F by I at position 111, F by V at position 111, R by H at position 113, R by Q at position 113, L by V at position 116, L by I at position 116, L by T at position 116, L by Q at position 116, L by H at position 116, L by A at position 116, L by V at position 120, L by I at position 120, L by T at position 120, L by Q at position 120, L by H at position 120, L by A at position 120, K by Q at position 123, K by T at position 123, K by S at position 123, K by H at position 123, R by H at position 124, R by Q at position 124, R by H at position 128, R by Q at position 128, L by V at position 130, L by I at position 130, L by T at position 130, L by Q at position 130, L by H at position 130, L by A at position 130, K by Q at position 134, K by T at position 134, K by S at position 134, K by H at position 134, K by Q at position 136, K by T at position 136, K by S at position 136, K by H at position 136, E by Q at position 137, E by H at position 137, Y by H at position 163, Y by I at position 163I, R by H at position 165, R by Q at position 165.

By 2D-scanning: M by V at position 1, M by I at position 1, M by T at position 1, M by Q at position 1, M by A at position 1, L by V at position 5, L by I at position 5, L by T at position 5, L by Q at position 5, L by H at position 5, L by A at position 5, F by I at position 8, F by V at position 8, L by V at position 9, L by I at position 9, L by T at position 9, L by Q at position 9, L by H at position 9, L by A at position 9, R by H at position 11, R by Q at position 11, F by I at position 15, F by V at position 15, K by Q at position 19, K by T at position 19, K by S at position 19, K by H at position 19, W by S at position 22, W by H at position 22, N by H at position 25, N by S at position 25, N by Q at position 25, R by H position 27, R by Q position 27, L by V at position 28, L by I at position 28, L by T at position 28, L by Q at position 28, L by H at position 28, L by A at position 28, E by Q at position 29, E by H at position 29, Y by H at position 30, Y by I at position 30, L by V at position 32, L by I at position 32, L by T at position 32, L by Q at position 32, L by H at position 32, L by A at position 32, K by Q at position 33, K by T at position 33, K by S at position 33, K by H at position 33, R by H at position 35, R by Q at position 35, M by V at position 36, M by I at position 36, M by T at position 36, M by Q at position 36, M by A at position 36, D by Q at position 39, D by H at position 39, D by G at position 39, E by Q at position 42, E by H at position 42, K by Q at position 45, K by T at position 45, K by S at position 45, K by H at position 45, L by V at position 47, L by I at position 47, L by T at position 47, L by, Q at position 47, L by H at position 47, L by A at position 47, K by Q at position 52, K by T at position 52, K by S at position 52, K by H at position 52, F by I at position 67, F by V at position 67, R by H at position 71, R by Q at position 71, D by Q at position 73, D by H at position 73, D by G at position 73, E by Q at position 81, E by H at position 81, E by Q at position 85, E by H at position 85, Y by H at position 92, Y by I at position 92, K by Q at position 99, K by T at position 99, K by S at position 99, K by H at position 99, E by Q at position 103, E by H at position 103, E by Q at position 104, E by H at position 104, K by Q at position 105, K by T at position 105, K by S at position 105, K by H at position 105, E by Q at position 107, E by H at position 107, K by Q at position 108, K by T at position 108, K by S at position 108, K by H at position 108, E by Q at position 109, E by H at position 109, D by Q at position 110, D by H at position 110, D by G at position 110, F by I at position 111, F by V at position 111, R by H at position 113, R by Q at position 113, L by V at position 116, L by I at position 116, L by T at position 116, L by Q at position 116, L by H at position 116, L by A at position 116, L by V at position 120, L by I at position 120, L by T at position 120, L by Q at position 120, L by H at position 120, L by A at position 120, K by Q at position 123, K by T at position 123, K by S at position 123, K by H at position 123, R by H at position 124, R by Q at position 124, R by H at position 128, R by Q at position 128, L by V at position 130, L by I at position 130, L by T at position 130, L by Q at position 130, L by H at position 130, L by A at position 130, K by Q at position 134, K by T at position 134, K by S at position 134, K by H at position 134, K by Q at position 136, K by T at position 136, K by S at position 136, K by H at position 136, E by Q at position 137, E by H at position 137, Y by H at position 138, Y by I at position 138, R by H at position 152, R by Q at position 152, Y by H at position 155, Y by I at position 155, R by H at position 159, R by Q at position 159, Y by H at position 163, Y by I at position 163, R by H at position 165, R by Q at position 165, M by D at position 1, M by E at position 1, M by K at position 1, M by N at position 1, M by R at position 1, M by S at position 1, L by D at position 5, L by E at position 5, L by K at position 5, L by N at position 5, L by R at position 5, L by S at position 5, L by D at position 6, L by E at position 6, L by K at position 6, L by N at position 6, L by R at position 6, L by S at position 6, L by Q at position 6, L by T at position 6, F by E at position 8, F by K at position 8, F by R at position 8, F by D at position 8, L by D at position 9, L by E at position 9, L by K at position 9, L by N at position 9, L by R at position 9, L by S at position 9, Q by D at position 10, Q by E at position 10, Q by K at position 10, Q by N at position 10, Q by R at position 10, Q by S at position 10, Q by T at position 10, S by D at position 12, S by E at position 12, S by K at position 12, S by R at position 12, S by D at position 13, S by E at position 13, S by K at position 13, S by R at position 13, S by N at position 13, S by Q at position 13, S by T at position 13, N by D at position 14, N by E at position 14, N by K at position 14, N by Q at position 14, N by R at position 14, N by S at position 14, N by T at position 14, F by D at position 15, F by E at position 15, F by K at position 15, F by R at position 15, Q by D at position 16, Q by E at position 16, Q by K at position 16, Q by N at position 16, Q by R at position 16, Q by S at position 16, Q by T at position 16, C by D at position 17, C by E at position 17, C by K at position 17, C by N at position 17, C by Q at position 17, C by R at position 17, C by S at position 17, C by T at position 17, L by N at position 20, L by Q at position 20, L by R at position 20, L by S at position 20, L by T at position 20, L by D at position 20, L by E at position 20, L by K at position 20, W by D at position 22, W by E at position 22, W by K at position 22, W by R at position 22, Q by D at position 23, Q by E at position 23, Q by K at position 23, Q by R at position 23, L by D at position 24, L by E at position 24, L by K at position 24, L by R at position 24, W by D at position 79, W by E at position 79, W by K at position 79, W by R at position 79, N by D at position 80, N by E at position 80, N by K at position 80, N by R at position 80, T by D at position 82, T by E at position 82, T by K at position 82, T by R at position 82, I by D at position 83, I by E at position 83, I by K at position 83, I by R at position 83, I by N at position 83, I by Q at position 83, I by S at position 83, I by T at position 83, N by D at position 86, N by E at position 86, N by K at position 86, N by R at position 86, N by Q at position 86, N by S at position 86, N by T at position 86, L by D at position 87, L by E at position 87, L by K at position 87, L by R at position 87, L by N at position 87, L by Q at position 87, L by S at position 87, L by T at position 87, A by D at position 89, A by E at position 89, A by K at position 89, A by R at position 89, N by D at position 90, N by E at position 90, N by K at position 90, N by Q at position 90, N by R at position 90, N by S at position 90, N by T at position 90, V by D at position 91, V by E at position 91, V by K at position 91, V by N at position 91, V by Q at position 91, V by R at position 91, V by S at position 91, V by T at position 91, Q by D at position 94, Q by E at position 94, Q by Q at position 94, Q by N at position 94, Q by R at position 94, Q by S at position 94, Q by T at position 94, I by D at position 95, I by E at position 95, I by K at position 95, I by N at position 95, I by Q at position 95, I by R at position 95, I by S at position 95, I by T at position 95, H by D at position 97, H by E at position 97, H by K at position 97, H by N at position 97, H by Q at position 97, H by R at position 97, H by S at position 97, H by T at position 97, L by D at position 98, L by E at position 98, L by K at position 98, L by N at position 98, L by Q at position 98, L by R at position 98, L by S at position 98, L by T at position 98, V by D at position 101, V by E at position 101, V by K at position 101, V by N at position 101, V by Q at position 101, V by R at position 101, V by S at position 101, V by T at position 101, M by C at position 1, L by C at position 6, Q by C at position 10, S by C at position 13, Q by C at position 16, L by C at position 17, V by C at position 101, L by C at position 98, H by C at position 97, Q by C at position 94, V by C at position 91, N by C at position 90. Since modifications will be apparent to those of skill in this art, it is intended that this invention be limited only by the scope of the appended claims.

1. A method for increasing serum stability of therapeutic target proteins, comprising modifying a target protein to exhibit increased resistance to protease digestion, thereby increasing serum stability of the target protein.

2. The method of claim 1, wherein modification of the protein is effected by:
selecting, on the target protein, target amino acids susceptible to proteolysis;
replacing each target amino acid with a replacement amino acid amenable to decreased proteolysis to form a candidate lead protein, wherein one amino acid replacement occurs on each target protein;
expressing each candidate lead protein in a cell contained in an addressable array; and
assaying each candidate lead protein for the presence of increased resistance to proteolysis, thereby identifying proteins that are Leads, wherein Leads exhibiting increased resistance to proteolysis have increased serum half-life compared to the wild-type target protein.

3. The method of claim 2, wherein the target protein is a cytokine.

4. The method of claim 3, wherein the cytokine is IFNα-2b and IFN-β.

5. The method of claim 2, wherein:
the selection of the one or more target amino acids is conducted in silico and the target amino acids are designated is-HITS.

6. The method of claim 5, wherein:
in silico selection further comprises selecting an is-HIT target residue that is susceptible to digestion by one or more proteases.

7. The method of claim 6, wherein the Leads possess increased resistance to proteolysis compared to the wild-type target protein.

8. The method of claim 2, wherein the method is repeated and more than one amino acid replacement occurs on each target protein.

9. The method of claim 1, wherein modification of the protein is effected by:
identifying, within a target protein or peptide, target amino acids amenable to providing resistance to proteolysis upon amino acid replacement, wherein each target amino acid is designated as an in silico-HIT (is-HIT);
identifying one or more replacement amino acids, specific for each is-HIT, amenable to providing the resistance to proteolysis to the target upon amino acid replacement of the target protein or peptide, wherein each single amino acid replacement within the target protein or peptide is designated as a candidate lead protein;
producing a population of sets of nucleic acid molecules that encode each of the candidate lead proteins, wherein each candidate lead protein comprises a single amino acid replacement, and wherein each polynucleotide in a set encodes a candidate lead protein that differs by one amino acid from the target protein or peptide;
introducing each set of nucleic acid molecules into host cells and expressing the encoded candidate lead proteins, wherein the host cells are present in an addressable array; and
individually screening the sets of encoded candidate lead proteins to identify one or more proteins that has activity that differs from the target protein, wherein each such protein is designated a Lead mutant protein.

10. The method of claim 9, wherein the target protein is a cytokine.

11. The method of claim 10, wherein the cytokine is IFNα-2b and IFN-β.

12. The method of claim 9, wherein:
the nucleic acid molecules are produced by site-specific mutagenesis.

13. The method of claim 9, further comprising:
recombining the nucleic acid molecules that encode the Leads, to produce a set of candidate SuperLeads comprising a combination of two or more single amino acid mutations derived from two or more LEAD mutant proteins;
introducing the each set of candidate SuperLeads into cells; and
individually screening the sets of cells that encode the candidate SuperLeads to identify one or more cells that encodes a protein that has activity that differs from the target protein and has properties that differ from the original Leads, wherein each such protein is designated a SuperLead.

14. The method of claim 13, wherein:
the nucleic acid molecules are produced by a method selected from among Additive Directional Mutagenesis, multi-overlapped primer extensions, oligonucleotide-mediated mutagenesis, nucleic acid shuffling, recombination, site-specific mutagenesis and de novo synthesis.

15. The method of claim 13, wherein the recombining is two, three or more up to all of the nucleic acids encoding the Leads.

16. The method of claim 9, wherein each is-HIT target amino acid is susceptible to digestion by one or more proteases.

17. The method of claim 16, wherein the Leads or SuperLeads possess increased resistance to proteolysis compared to the wild-type target protein.

18. The method of claim 9, wherein the method is repeated and more than one amino acid replacement occurs on each target protein.
